(19) United States
(12) Patent Application Publication
(10) Pub. No.: US 2026/0234147 A1
(43) Pub. Date: Aug. 13, 2026

(54) COMPOUNDS FOR TREATING OPIOID MISUSE

(71) Applicant: University of Florida Research Foundation, Incorporated, Gainesville, FL (US)

(72) Inventors: Christopher R. McCurdy, Gainesville, FL (US); Morgan Leigh Crowley, Gainesville, FL (US); Sushobhan Mukhopadhyay, Gainesville, FL (US); Sampa Gupta, Gainesville, FL (US)

(73) Assignee: University of Florida Research Foundation, Incorporated, Gainesville, FL (US)

(21) Appl. No.: 19/154,461

(22) PCT Filed: Feb. 7, 2024
(86) PCT No.: PCT/US2024/014804

§ 371 Date: Aug. 7, 2025

Related U.S. Application Data
(60) Provisional application No. 63/444,010, filed on Feb. 8, 2023.
(60) Provisional application No. 63/520,086, filed on Aug. 16, 2023.

(51) Int. Cl. C07D 471/14; A61K 31/437; A61K 31/4375; A61K 31/444; A61K 31/496; A61K 31/5375; A61K 31/541; A61K 31/55; C07B 59/00; C07D 401/06; C07D 455/02

(52) U.S. Cl. CPC C07D 471/14; A61K 31/437; A61K 31/4375; A61K 31/444; A61K 31/496; A61K 31/5375; A61K 31/541; A61K 31/55; C07B 59/002; C07D 401/06; C07D 455/02; C07B 2200/05

(57) ABSTRACT

Described herein are new small molecules that bind to opioid receptors and/or adrenergic receptors.

### RELATED APPLICATIONS

This application claims priority under 35 U.S.C. § 119(e) to United States provisional patent applications, U.S. Ser. No. 63/444,010, filed Feb. 8, 2023; and U.S. Ser. No. 63/520,086, filed Aug. 16, 2023, the entire contents of each of which are incorporated herein by reference.

### STATEMENT OF GOVERNMENT SUPPORT

This invention was made with government support under grant RO1 DA047855 awarded by the National Institutes of Health. The government has certain rights in the invention.

### BACKGROUND

Opioid preparations have been utilized to manage pain since the earliest records of human existence. Morphine, the major opiate alkaloid, was discovered and purified in 1804 by the German pharmacist, Friedrich Serturner. Since that time, morphine and other alkaloids (e.g. codeine) from the opium poppy have served as prescription medicines for the treatment of moderate to severe pain.

Unfortunately, the naturally occurring alkaloids as well as the semi-synthetic alkaloids have more recently been over prescribed resulting in the current opioid epidemic in the United States. Each day, 115 individuals die from opioid overdose due to the resultant severe respiratory depression induced by these agents at high doses. Of course, it is not only prescription opioids that are problematic, illicit opioids have also made a substantial comeback. Heroin (diacetyl morphine), black tar heroin, fentanyl, and synthetic derivatives thereof, have become more widely available through illegitimate channels. The result is evident in the unprecedented number of individuals that have become addicted and now suffer from the chronic disease of addiction.

Effective medication interventions are available, such as the widely utilized opioid agonists, methadone and buprenorphine as well as the antagonists, naloxone and naltrexone. However, these have not been successful in addressing the problem mostly because they are highly underutilized. In addition, they are medical interventions that are considered replacement therapies in the case of methadone and buprenorphine. Yet, their pharmacokinetic and pharmacodynamics profiles render them not as euphoric as other opioids. Still, there are issues with these medications that cause undesirable side effects. Moreover, when an individual discontinues these medications they can go into withdrawal. Withdrawal intensity varies from person to person and is usually not life threatening. During detoxification, the withdrawal symptoms can be intense and often times lead to relapse.

Two medications for treating opioid withdraw include lofexidine and clonidine. Both are utilized to suppress withdrawal and act as α₂ adrenergic agonists. Clonidine and lofexidine act to reduce physical withdrawal and anxiety as well as possibly reducing cravings. For this reason, some physicians prescribe clonidine or lofexidine instead of buprenorphine, or in combination with buprenorphine. There is a role that the adrenergic system plays in the intervention of opioid addiction treatment, but this has not been addressed in a dual-acting medication.

### SUMMARY OF THE INVENTION

Although the opium poppy is the most well-known and studied plant with opioid activity, other plants are known to exist that also possess opioid activity. Among these is Mitragyna speciosa, more commonly known as kratom. Kratom has been utilized as a traditional herbal medicine in Thailand and Malaysia for centuries to improve stamina for hard laborers, to treat pain, and importantly, to serve as a substitute for opium to avoid withdrawal when opium was not available. Moreover, kratom has been employed as a tool to ween addicts from opium or other opioids (such as heroin).

Interestingly, mitragynine, the most abundant alkaloid in kratom, has a “dirty” pharmacological profile. It is reported to bind to mu and kappa opioid receptors, but also adenosine, serotonin, dopamine, and α₂ adrenergic receptors. This unique combination of receptor action is interesting due to the fact that all of these systems have been implicated in withdrawal. The fact that mitragynine interacts with opioid and α₂ adrenergic receptors provides a strong scientific premise for how kratom, more specifically mitragynine, might aid in the alleviation of withdrawal symptoms since these two receptor systems are targeted with individual drugs to treat opioid dependence and withdrawal. Additionally, the fact that mitragynine is a G-protein biased partial agonist, may explain, in part, why it does not result in severe respiratory depression. Indeed, there have been no reports of deaths linked to kratom in its traditional use.

There is a strong concern that kratom products available in the United States are not safe. Rightfully so, the US Food and Drug Administration has warned against the use of kratom. This has been underscored with the recent Salmonella outbreak that resulted in the FDA issuing a mandatory recall order. Although there are kratom vendors that test their raw materials for bacterial, yeast, and mold, there is little enforcement of this testing. Therefore, studying the major alkaloid, mitragynine, purified from extracts is a logical way to investigate the ability for it to serve as a potential therapeutic.

Described herein are mitragynine analogs that provide an alternative therapeutic for treatment of opioid use disorders.

In one aspect, provided herein are compounds of Formula (I′):

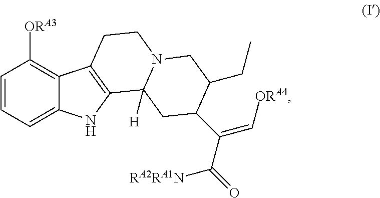

or pharmaceutically acceptable salt thereof, wherein R^{A1}, R^{A2}, R^{A3}, and R^{A4} are as defined herein.

In a further aspect, provided herein are compounds of Formula (I):

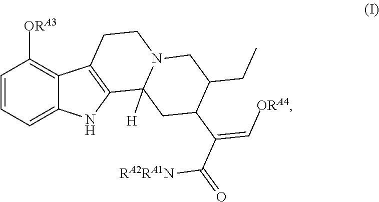

or pharmaceutically acceptable salt thereof, wherein R^{A1}, R^{A2}, R^{A3}, and R^{A4} are as defined herein.

In another aspect, provided herein are compounds of Formula (II):

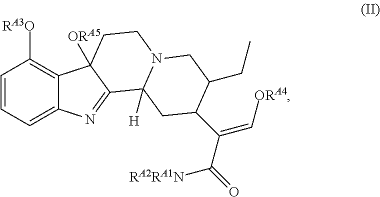

or pharmaceutically acceptable salt thereof, wherein R^{A1}, R^{A2}, R^{A3}, R^{A4}, and R^{A5} are as defined herein.

In a further aspect, provided herein are compounds of Formula (III):

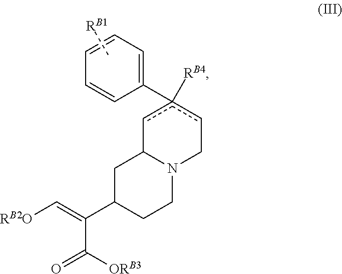

or a pharmaceutically acceptable salt thereof, wherein R^{B1}, R^{B2}, R^{B3}, and R^{B4} are as defined herein.

In another aspect, provided herein are compounds of Formula (IV):

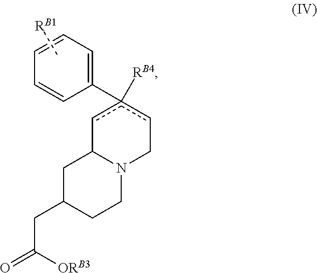

or a pharmaceutically acceptable salt thereof, wherein R^{B1}, R^{B3}, and R^{B4} are as defined herein.

In a further aspect, provided herein are compounds of Formula (V):

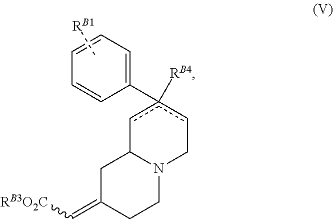

or a pharmaceutically acceptable salt thereof, wherein R^{B1}, R^{B3}, and R^{B4} are as defined herein.

In another aspect, provided herein are compounds of Formula (VI):

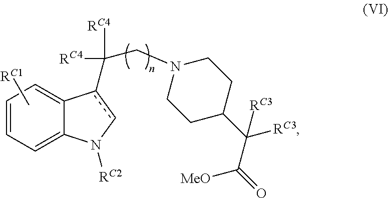

or pharmaceutically acceptable salt thereof, wherein R^{C1}, R^{C2}, R^{C3}, R^{C4}, and n are as defined herein.

In a further aspect, provided herein are compounds of Formula (VII):

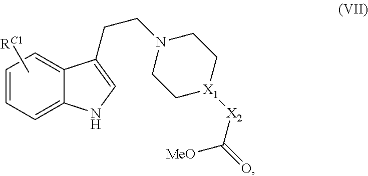

or pharmaceutically acceptable salt thereof, wherein R^{C1}, X₁, and X₂ are as defined herein.

In an additional aspect, provided herein are compounds of Formula (VIII):

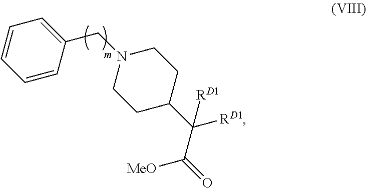

or pharmaceutically acceptable salt thereof, wherein R^{D1} and m are as defined herein.

In a further aspects, provided herein are compounds of Formula (IX):

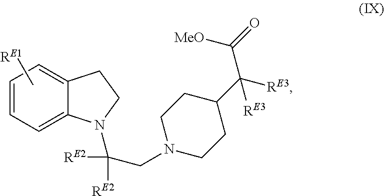

or pharmaceutically acceptable salt thereof, wherein R^{E1}, R^{E2}, and R^{E3} are as defined herein.

In another aspect, provided herein are compositions comprising a compound provided herein (e.g., a compound of Formula (I′), (I), (II), (III), (IV), (V), (VI), (VII), (VIII), (IX)), or pharmaceutically acceptable salt thereof, and a pharmaceutically acceptable excipient

In an additional aspect, the present disclosure provides methods of treating or preventing substance intake by a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In another aspect, provided herein are methods of treating or preventing a substance use disorder in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In one aspect, the present disclosure provides methods of treating the symptoms of substance use disorder in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In an additional aspect, the present disclosure provides methods of treating or preventing substance addiction by a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In another aspects, provided herein are methods of treating the symptoms of substance addiction by a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In one aspect, the present disclosure provides methods of treating or preventing neurotoxic effects resulting from substance use disorder, substance addiction, and/or substance intake in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing a disease or disorder associated with one or more opioid receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing a disease or disorder associated with one or more adrenergic receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing substance withdrawal comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing symptoms associated with substance withdrawal comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing substance dependence comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In one aspect, the present disclosure provides a kit comprising: a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)); and instructions for administering the compound, or pharmaceutically acceptable salt thereof, or composition to a subject.

Additional aspects of any of the above methods are those wherein the subject is in need of such treatment, and those wherein the subject is identified as in need of such treatment.

The details of certain embodiments of the disclosure are set forth in the Detailed Description of Certain Embodiments, as described below. Other features, objects, and advantages of the disclosure will be apparent from the Definitions, Figures, Examples, and Claims. It should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

FIG. 1 shows a graph of compounds 3-21 and 3-24 after screening for in vitro efficacy using a [³⁵S]GTPγS functional assay in Chinese hamster ovary (CHO) cells stably expressing the human MOR. In this assay, compound 3-21 failed to produce agonist-like effects up to 10 mM at MOR, whereas compound 3-24 produced low, but measurable, partial agonist activity at MOR (Eₘₐₓ=24%). Compound 3-21 was also evaluated for its antagonistic properties against a concentration of DAMGO equal to 10× its Kᵢ (MOR) value. In this assay, compound 3-21 produced an 18-fold rightward shift in the DAMGO concentration-effect curve, comparable to that of MG. These results suggest that compound 3-21 is a weak antagonist at MOR.

FIGS. 2A-2D shows the in vivo assessment of compound 3-21 (10-32 mg/kg, i.p.) in a drug discrimination assay, hotplate assay, and rectal temperature measurement using Sprague-Dawley rats trained to discriminate 3.2 mg/kg of morphine from vehicle. FIG. 2A shows the percent of drug lever responding. FIG. 2B shows the percent of vehicle response rate. FIG. 2C shows an assessment of the antinociceptive activity of compound 3-21 in a 52° C. hot-plate assay. FIG. 2D shows the measurement of the change in rectal temperature by compound 3-21. Sprague-Dawley rats were trained to discriminate the MOR agonist, morphine (3.2 mg/kg, i.p.). In this assay, Compound 3-21 (up to 17.8 mg/kg, i.p.) produced 52% of morphine-lever responding and decreased the response rate at the highest dose test (32 mg/kg), which did not produce full substitution, similar to the scaffold kratom alkaloid, MG (FIG. 2A, 2B).

FIG. 3 shows a graph assessment of the antinociceptive and hypothermic effects of compound 3-24 in naïve Sprague-Dawley rats. The hotplate assay was assessed at 52° C. Rectal temperature measured immediately after the hot plate assay. The MOR antagonist, naltrexone (0.1 mg/kg), was administered at the end of assessment of each test compound alone.

FIG. 4 shows compound SUSM 501 is partial agonist.

FIG. 5 shows compound SUSM 524 acting as agonist.

FIG. 6 shows compound SUSM 522 acting as antagonist.

FIG. 7 shows compound SUSM 523 acting as antagonist.

FIG. 8 shows compound SUSM 528 acting as agonist, but less potent than DAMGO.

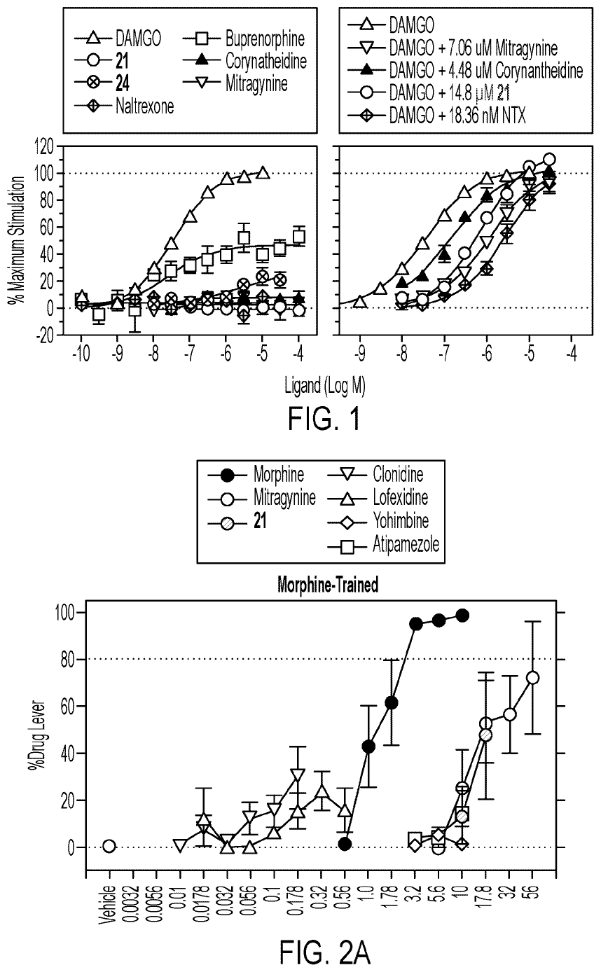

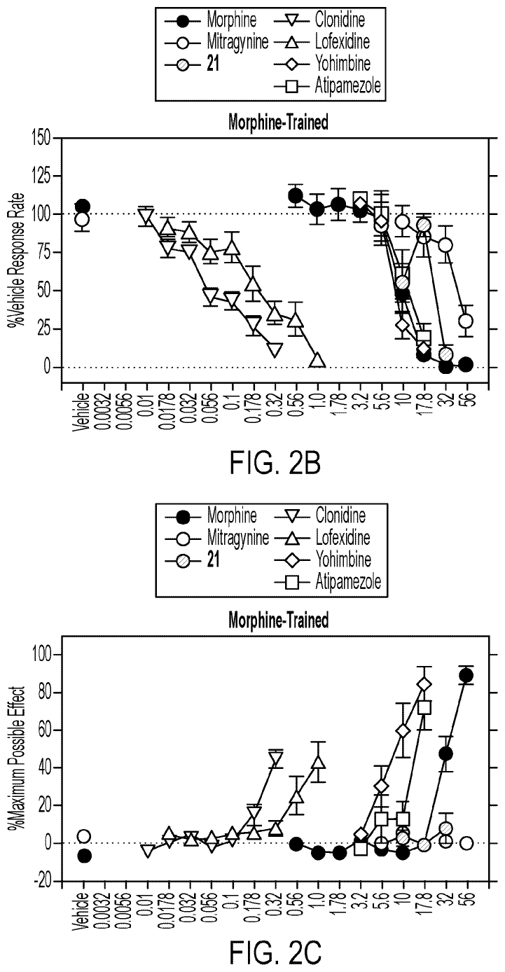

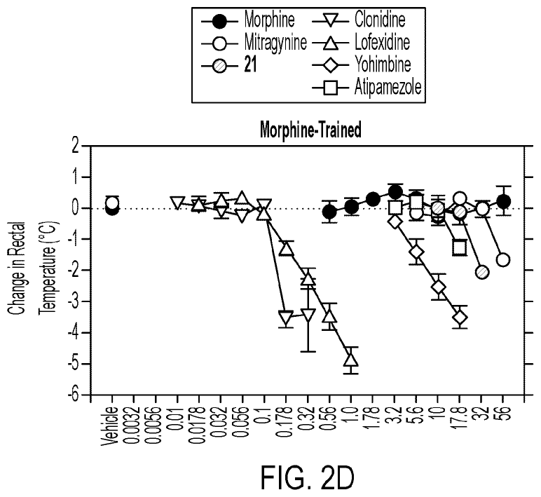

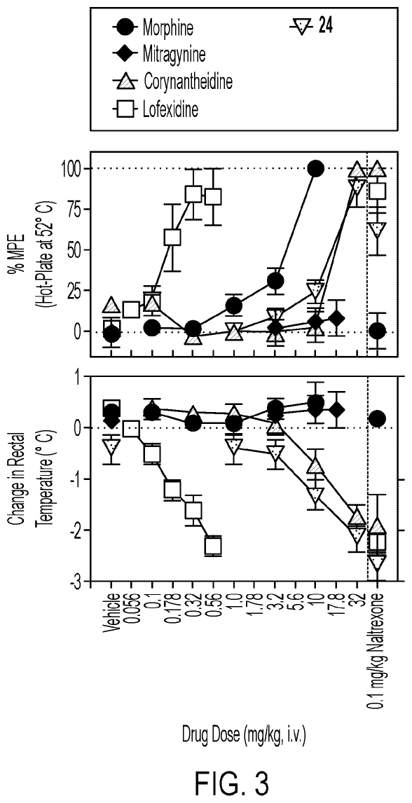

### DEFINITIONS

For convenience, certain terms employed herein, in the specification, examples and appended claims are collected herein.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds and oligonucleotides described herein, even though only a single tautomeric form may be represented. All such isomeric forms of such compounds herein are expressly included in the present disclosure. The term “isomers” is intended to include diastereoisomers, enantiomers, regioisomers, structural isomers, rotational isomers, tautomers, and the like. For compounds that contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₅), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁-4 haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CC13, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

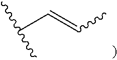

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁-20 alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has Ito 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁. 8 alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and lor 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁-20 alkynyl. In certain embodiments, the heteroalkynyl group is a substituted heteroC₁-20 alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen.

The term “thiol” or “thio” refers to the group —SH. The term “substituted thiol” or “substituted thio,” by extension, refers to a thiol group wherein the sulfur atom directly attached to the parent molecule is substituted with a group other than hydrogen.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen. The term “disubstituted amino” refers to an amino group wherein the nitrogen atom directly attached to the parent molecule is substituted with two groups other than hydrogen. The term “trisubstituted amino” refers to an amino group wherein the nitrogen atom directly attached to the parent molecule is substituted with three non-hydrogen groups.

Affixing the suffix “-ene” to a group indicates the group is a divalent moiety, e.g., alkylene is an optionally substituted divalent moiety of alkyl (e.g., unsubstituted C₆ alkylene is represented by —(CH₂)₆—) and heteroalkylene is the divalent moiety of heteroalkyl (e.g., unsubstituted hetero-C₆-alkylene is represented by, for example, —(CH₂)—O—(CH₂)₅—).

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary substituents include halogen, —CN, —NO₂, —N₃, —SO₂H, —SO₃H, —OH, —OR—, —ON(R^{bb})₂, —N(R^{bb})₂, —N(R^{bb})₃⁺X—, —SH, —SR—, —C(═O)H, —C(═O)R^{aa}, —CO₂H, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R—, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R—, —C(═O)SR^{aa}, —SC(═O)SR^{aa}, —OC(═O)SR^{aa}, —SC(═O)OR^{aa}, —SC(═O)R^{aa}, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl;
or two geminal hydrogens on a carbon atom are replaced with the group ═O or =S, wherein:
   each instance of R^{aa} is, independently, selected from C₁₋₆ alkyl, C₁₋₆ perhaloalkyl, C₁₋₆ alkenyl, C₁₋₆ alkynyl, heteroC₁₋₆ alkyl, heteroC₁₋₆alkenyl, heteroC₁₋₆alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —C(═O)R^{aa}, —C(═O)N(R^{cc})₂, —CO₂R^{aa}, —SO₂R^{cc}, —SO₂N(R^{cc})₂, —SO₂R^{cc}, —SO₂OR^{cc}, —SOR^{aa}, —C(═O)SR^{cc}, C₁₋₆ alkyl, C₁₋₆ perhaloalkyl, C₁₋₆ alkenyl, C₁₋₆ alkynyl, heteroC₁₋₆alkyl, heteroC₁₋₆alkenyl, heteroC₁₋₆alkynyl, C₃₋₁₀ carbocyclyl, 3-14 membered heterocyclyl, C₆₋₁₄ aryl, and 5-14 membered heteroaryl;
   each instance of R^{cc} is, independently, selected from hydrogen, C₁₋₆ alkyl, C₁₋₆ perhaloalkyl, C₁₋₆ alkenyl, C₁₋₆ alkynyl, heteroC₁₋₆ alkyl, heteroC₁₋₆ alkenyl, heteroC₁₋₆ alkynyl, C₃₋₁₀ carbocyclyl, 3-14 membered heterocyclyl, C₆₋₁₄ aryl, and 5-14 membered heteroaryl; and
   each X⁻ is a counterion.

In certain embodiments, each substituent is independently halogen, substituted or unsubstituted C₁₋₆ alkyl, —OR^{aa}, —SR—, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R—, or —NR^{bb}C(═O)N(R^{bb})₂. In certain embodiments, each substituent is independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R—, —NR^{bb}CO₂R—, or —NR^{bb}C(═O)N(R^{bb})₂, wherein R^{aa} is hydrogen, substituted or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each substituent is independently halogen, substituted or unsubstituted C₁₋₆ alkyl, —OR^{aa}, —N(R^{bb})₂, —CN, or —NO₂.

In certain embodiments, each substituent is independently halogen, substituted or unsubstituted C₁₋₁₀ alkyl, —OR^{aa}, —SR—, —N(R^{bb})₂, —CN, —SCN, or —NO₂, wherein R^{aa} is hydrogen, substituted or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, each nitrogen atom substituent is independently substituted or unsubstituted C₁₋₆ alkyl, —C(═O)R^{aa}, —CO₂R^{aa}, —C(═O)N(R^{bb})₂, or a nitrogen protecting group.

Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group.

Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{aa})₃, —P(R^{cc})₂, —P(R^{cc})₃⁺X—, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, at least one oxygen protecting group is silyl, TBDPS, TBDMS, TIPS, TES, TMS, MOM, THP, t-Bu, Bn, allyl, acetyl, pivaloyl, or benzoyl.

The term “silyl” refers to the group —Si(R′)₃, wherein R^{aa} is as defined herein.

In certain embodiments, each sulfur atom substituent is independently substituted or unsubstituted C₁₋₁₀ alkyl, —C(═O)R^{aa}, —CO₂R^{aa}, —C(═O)N(R^{bb})₂, or a sulfur protecting group.

In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{cc})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N+(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In certain embodiments, the subject is a human with a substance abuse disorder, substance addiction disorder, substance withdrawal disorder, neurological disorder, psychiatric disorder, painful condition, or opioid receptor mediated disorder, or symptoms thereof.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations). In certain embodiments, an effective amount is the combined amounts of a compound described herein in multiple doses. In certain embodiments, an effective amount is an amount sufficient to reduce symptoms associated with a neurological disorder, psychiatric disorder, painful condition, or opioid receptor mediated disorder. In certain embodiments, an effective amount is an amount sufficient to reduce symptoms associated with an addiction. In certain embodiments, an effective amount is an amount sufficient to treat a subject with a neurological disorder, psychiatric disorder, painful condition, or opioid receptor mediated disorder. In certain embodiments, an effective amount is an amount sufficient to treat an addiction.

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for binding opioid receptors (e.g., delta opioid receptors, mu opioid receptor, kappa opioid receptors). In certain embodiments, a therapeutically effective amount is an amount sufficient for binding adrenergic receptor (e.g., α₂ (e.g., alpha-2A and alpha-2C)). In certain embodiments, a therapeutically effective amount is an amount sufficient for treating stimulant (e.g., psychostimulant) abuse, addiction, or dependence, or other indications and diseases associated with opioid and/or adrenergic receptors.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of protein(s), for example, in the context of opioid receptors and/or adrenergic receptors, refers to a reduction in the activity of the protein. In some embodiments, the term refers to a reduction of the level of protein(s) activity, e.g., (opioid and/or adrenergic receptors) activity, to a level that is statistically significantly lower than an initial level, which may, for example, be a baseline level of protein activity. In some embodiments, the term refers to a reduction of the level of protein activity, e.g., opioid and/or adrenergic receptor activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of protein activity.

As used herein, “opioid receptor” refers to a group of inhibitory G protein-coupled receptors which bind opioids as ligands. Exemplary opioid receptors include delta opioid receptors, kappa opioid receptors, and mu opioid receptors. Opioid receptors bind opioid-like compounds and are responsible for mediating the effects of these compounds.

As used herein, “DOP,” “DOP,” “delta opioid receptor,” and “δ-opioid receptor” refer to an inhibitory 7-transmembrane G-protein coupled receptor coupled to the G protein Gᵢ/G₀. In humans, the delta opioid receptor is expressed in the brain including in the basal ganglia and neocortical regions. Enkephalins are endogenous ligands.

As used herein, “KOP,” “KOR,” “kappa opioid receptor,” and “x-opioid receptor” refer to a G protein-coupled receptor in humans encoded by OPRK1 gene. The receptor is coupled to the G protein Gᵢ/G₀. Dynorphin is the primary endogenous ligand.

As used herein, “MOP,” “MOR,” “mu opioid receptor,” and “μ-opioid receptor” refer to an inhibitory G-protein coupled receptor. It activates Gᵢ alpha subunit.

The term “adrenergic receptor” refers a class of G protein-coupled receptors. Exemplary receptor ligands include adrenaline and noradrenaline. There are two main groups, α and β. The receptors are divided into α₁ and α₂.

The terms “alpha-2 adrenergic receptor” and “α₂ adrenergic receptor” include G protein-coupled receptor (GPCR) associated with the Gᵢ heterotrimeric G-protein. The α₂ adrenergic receptors include α_{2A}, α_{2B}, and α_{2C}. The α_{2A} adrenergic receptors are located within the central nervous system including the brainstem, midbrain, hypothalamus, hippocampus, spinal cord, cerebral cortex, cerebellum, and septum. The α_{2B} adrenergic receptors are located within the central nervous system including within the midbrain, thalamus, amygdala, dorsal root ganglia, olfactory system, hippocampus, cerebral cortex, basal ganglia, substantia nigra, and ventral tegmentum.

The terms “substance abuse disorders” and “substance use disorder” are used interchangeably to refer to a mental disorder affecting a person's brain and behavior, resulting in the inability to control their use of substances, including psychoactive substances either licit or illicit (e.g., cocaine, methamphetamine, ecstasy, alcohol, opioids, stimulants, psychedelics). Severe substance use disorders may be referred to as addictions.

The terms “substance addiction” refers to severe forms of substance use disorders. Drug addiction is a progressive disease resulting in losing control of the use of one or more substances (e.g., drugs, pharmaceuticals/medications, stimulants) despite consequences of that use. A disease of the mind characterized by compulsive engagement in rewarding or addictive stimuli. An addiction often involves addictive stimuli that are reinforcing (e.g., increase the likelihood that a person will seek repeated exposure to the agent causing the stimulus) and intrinsically rewarding (e.g., they are perceived by a person as being inherently desirable, positive, and pleasurable). The addiction may arise through transcriptional or epigenetic mechanisms and generally develops over time as a result of persistent exposure to addictive stimulus or stimuli. Cognitive control, particularly inhibitory control over behavior, is impaired in a person suffering from addiction. Additionally, stimulus-driven behavioral responses (i.e., stimulus control) that are associated with a particular rewarding stimulus tend to dominate the behavior of a person suffering from addiction. The term addiction encompasses addiction to substances (e.g., cocaine, opioids, and the like), alcohol, gambling, etc. In certain embodiments, the addiction is a substance addiction. In certain embodiments, the addiction is an opioid addiction. In certain embodiments, the addiction is a prescription opioid addiction. In some embodiments, the addiction is a codeine, fentanyl, hydromorphone, meperidine, methadone, morphine, oxycodone, or oxymorphone addiction. In certain embodiments, the addiction is an illicit opioid addiction. In some embodiments, the addiction is a heroin addiction.

The terms “substance intake” refers to the consumption of substances (e.g., drugs, pharmaceuticals/medications, stimulants).

The term “substance” as used herein refers to psychoactive compound or molecule. Psychoactive compounds and molecules change nervous system function (i.e., brain function) and result in alterations in mood, awareness, thoughts, feelings, and/or behavior. As used herein, common substances include drugs, pharmaceuticals/medications, and other stimulants, including but not limited to, alcohol, caffeine, nicotine, marijuana, certain pain medicines, heroin, LSD, cocaine, and amphetamines (e.g., methamphetamine, 3,4-methylenedioxymethamphetamine, cathinone). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

The term “stimulant” refers to compounds/molecules that increase activity of the central nervous system and/or body, are pleasurable and invigorating, and/or have sympathomimetic effects.

The phrase “opioid receptor mediated disorder” refers to any disease or disorder caused by upregulation (e.g., increased function) or downregulation (e.g., decreased function) of opioid receptor function. An opioid receptor mediated disorder includes, in some embodiments, neurological disorders and psychiatric disorders.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, including, but not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome. In certain embodiments, a neurological disease that is addiction is preferred.

A “painful condition” includes, but is not limited to, neuropathic pain (e.g., peripheral neuropathic pain), central pain, deafferentation pain, chronic pain (e.g., chronic nociceptive pain, and other forms of chronic pain), post-operative pain (e.g., pain arising after hip, knee, or other replacement surgery), pre-operative pain, stimulus of nociceptive receptors (nociceptive pain), acute pain (e.g., phantom and transient acute pain), fibromyalgia, noninflammatory pain, inflammatory pain, pain associated with cancer, wound pain, burn pain, acute post-operative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from substance addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition. In certain embodiments, the painful condition is nociceptive pain. In certain embodiments, the painful condition is pain associated with withdrawal symptoms from substance addiction.

In certain embodiments, the painful condition is nociceptive pain. The term “nociceptive pain” refers to pain resulting from stimulation of nociceptive receptors. Without wishing to be bound by any particular theory, nociceptive pain may be caused by a chemical (e.g., capsaicin), mechanical (e.g., cutting), or thermal (e.g., hot or cold) stimulus. Nociceptive pain also includes visceral pain (i.e., pain that results from the activation of nociceptors. Nociceptive pain also includes pain resulting from the alteration of nociception as a result of alteration of opioid receptor function.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia). In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulation of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

In certain embodiments, the painful condition is inflammatory pain. In certain embodiments, the painful condition (e.g., inflammatory pain) is associated with an inflammatory condition and/or an immune disorder.

The term “psychiatric disorder” refers to a disease of the mind and includes diseases and disorders listed in the Diagnostic and Statistical Manual of Mental Disorders—Fifth Edition (DSM-IV), published by the American Psychiatric Association, Washington D. C. (2013). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

These and other exemplary substituents are described in more detail in the Detailed Description, Examples, and Claims. The invention is not limited in any manner by the above exemplary listing of substituents or definitions. Additional terms may be defined in other sections of this disclosure.

### DETAILED DESCRIPTION

Provided herein are compounds, compositions, methods, uses, and kits comprising compounds of Formula (I′), (I), (II), (III), (IV), (V), (VI), (VII), (VIII), and (IX). Compounds

In some embodiments, a compound described herein is an opioid receptor binder. In some embodiments, a compound described herein is an opioid receptor antagonist.

In some embodiments, a compound described herein is an α₂ adrenergic receptor binder. In some embodiments, a compound described herein is an α₂ adrenergic receptor antagonist.

In some embodiments, a compound described herein is both an opioid receptor binder and a α₂ adrenergic receptor binder. In certain embodiments, a compound described herein is both an opioid receptor antagonist and an α₂ adrenergic receptor antagonist.

### Compounds of Formula (I′), (I), and (II)

Provided herein are compounds of Formula (I′):

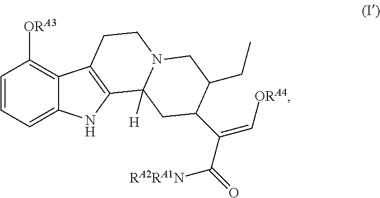

or pharmaceutically acceptable salt thereof, wherein:
R^{A1} is hydrogen, substituted or unsubstituted C₁₋₁₂ alkyl, —CD₃, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, —CD₃, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
   or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, —CD₃, or an oxygen protecting group; and
R^{A4} is hydrogen, substituted or unsubstituted C₁₋₁₂ alkyl, or an oxygen protecting group; provided the compound is not of the formula:

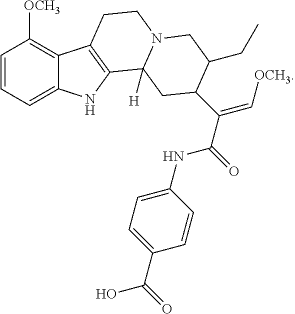 [SMILES: CCC1CN2CCc3c([nH]c4cccc(OC)c34)C2CC1/C(=C\OC)C(=O)Nc1ccc(C(=O)O)cc1]

In some embodiments, the compound of Formula (I′) is of Formula (I-A):

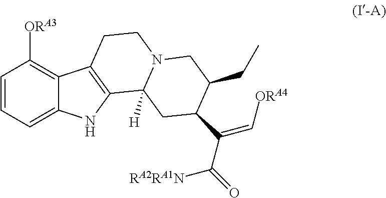

or pharmaceutically acceptable salt thereof.

In some embodiments, the compound of Formula (I′) is of Formula (I-B):

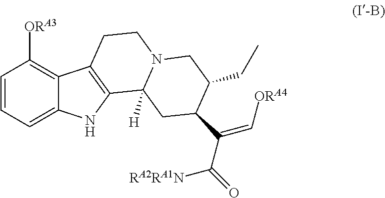

or pharmaceutically acceptable salt thereof.

In some embodiments, the compound of Formula (I′) is of formula:

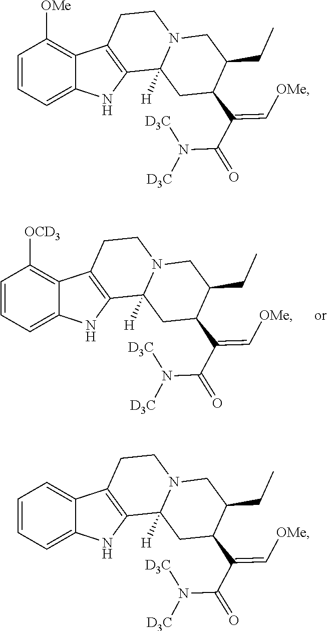

or pharmaceutically acceptable salt thereof.

Provided herein are compounds of Formula (I):

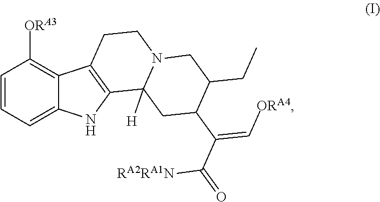

or pharmaceutically acceptable salt thereof, wherein: PGP
R^{A1} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
   or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, or an oxygen protecting group; and
R^{A4} is hydrogen, substituted or unsubstituted C₁₋₁₂ alkyl, or an oxygen protecting group;
   provided the compound is not of the formula:

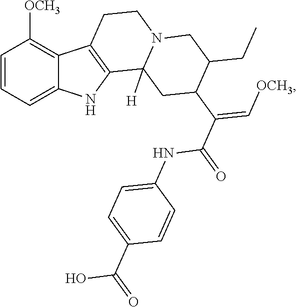 [SMILES: CCC1CN2CCc3c([nH]c4cccc(OC)c34)C2CC1/C(=C\OC)C(=O)Nc1ccc(C(=O)O)cc1]

In some embodiments, the compound of Formula (I) is of Formula (I-A):

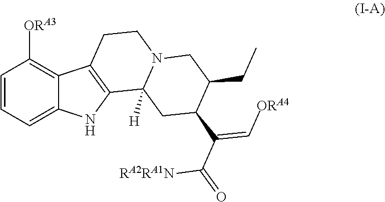

or pharmaceutically acceptable salt thereof. In some embodiments, the compound of Formula (I) is of formula:

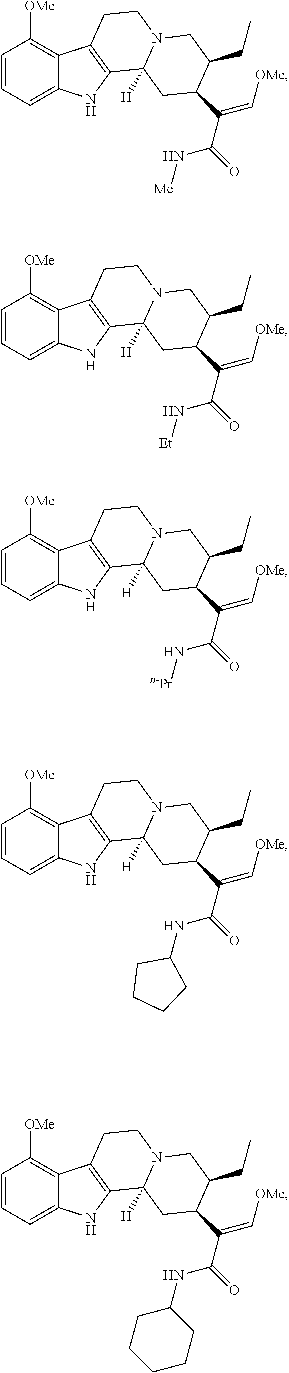

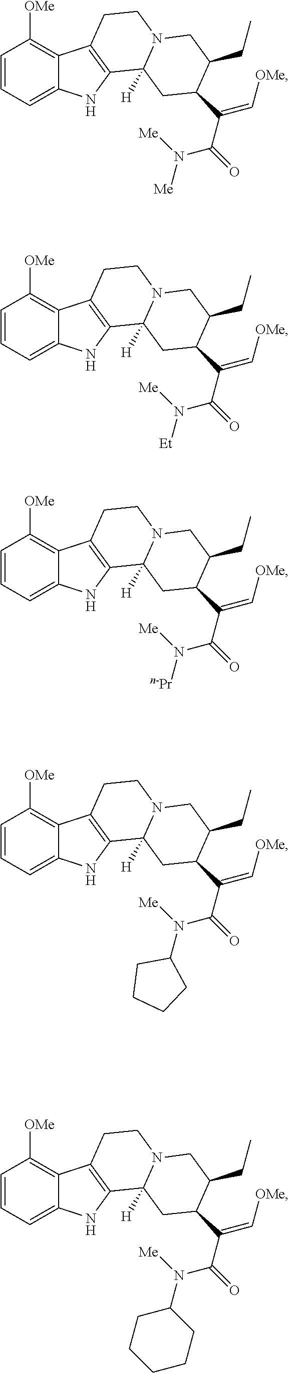

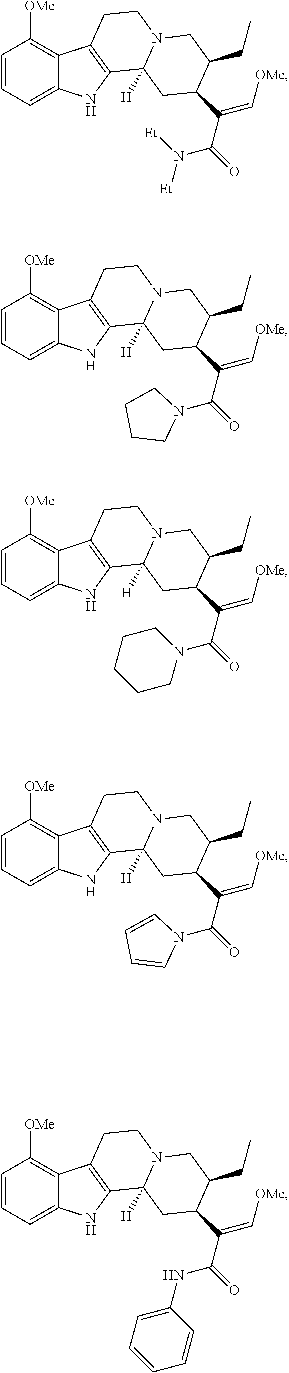 [SMILES: CC[C@@H]1CN2CCc3c([nH]c4cccc(OC)c34)[C@@H]2C[C@@H]1/C(=C\OC)C(=O)N(CC)CC ; CC[C@@H]1CN2CCc3c([nH]c4cccc(OC)c34)[C@@H]2C[C@@H]1/C(=C\OC)C(=O)N1CCCC1 ; CC[C@@H]1CN2CCc3c([nH]c4cccc(OC)c34)[C@@H]2C[C@@H]1/C(=C\OC)C(=O)N1CCCCC1 ; CC[C@@H]1CN2CCc3c([nH]c4cccc(OC)c34)[C@@H]2C[C@@H]1/C(=C\OC)C(=O)n1cccc1 ; CC[C@@H]1CN2CCc3c([nH]c4cccc(OC)c34)[C@@H]2C[C@@H]1/C(=C\OC)C(=O)Nc1ccccc1]

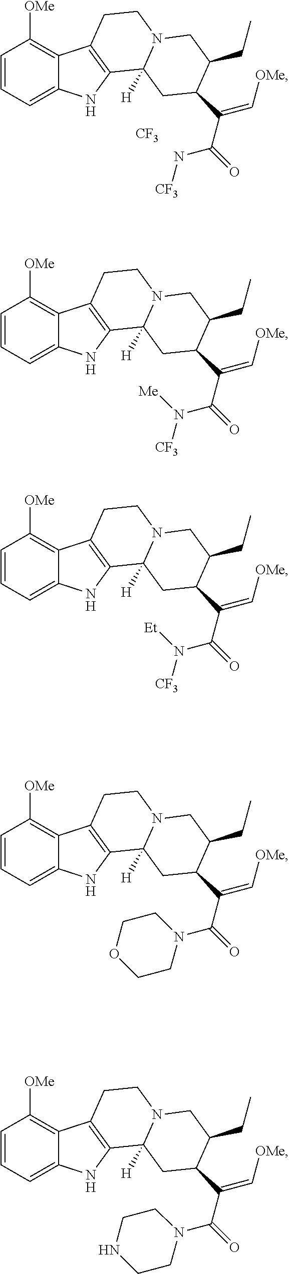

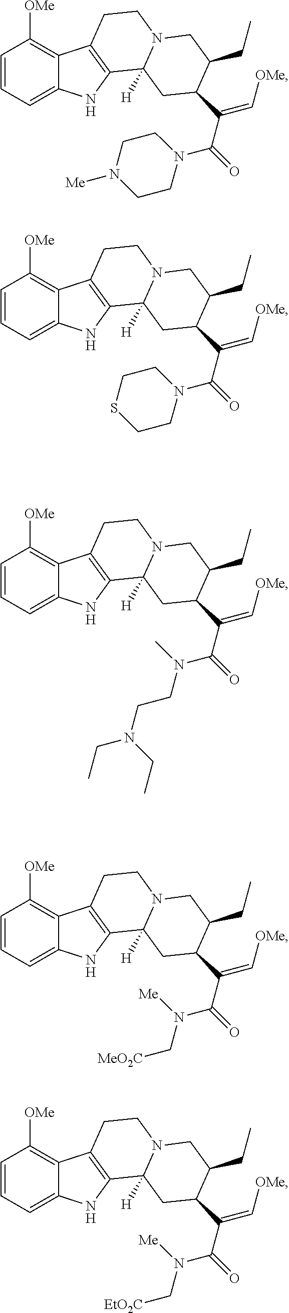 [SMILES: CC[C@@H]1CN2CCc3c([nH]c4cccc(OC)c34)[C@@H]2C[C@@H]1/C(=C\OC)C(=O)N1CCN(C)CC1 ; CC[C@@H]1CN2CCc3c([nH]c4cccc(OC)c34)[C@@H]2C[C@@H]1/C(=C\OC)C(=O)N1CCSCC1 ; CC[C@@H]1CN2CCc3c([nH]c4cccc(OC)c34)[C@@H]2C[C@@H]1/C(=C\OC)C(=O)N(C)CCN(CC)CC ; CC[C@@H]1CN2CCc3c([nH]c4cccc(OC)c34)[C@@H]2C[C@@H]1/C(=C\OC)C(=O)N(C)CC(=O)OC ; CCOC(=O)CN(C)C(=O)/C(=C/OC)[C@H]1C[C@H]2c3[nH]c4cccc(OC)c4c3CCN2C[C@H]1CC]

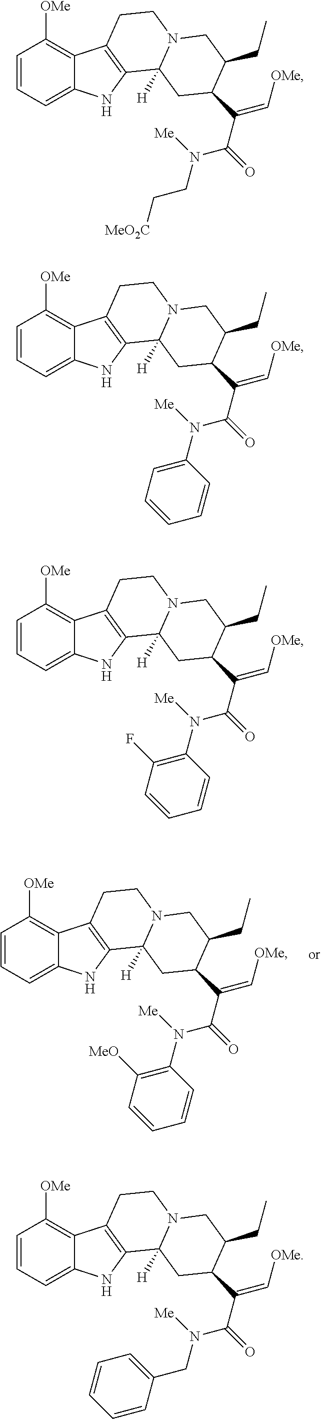 [SMILES: CC[C@@H]1CN2CCc3c([nH]c4cccc(OC)c34)[C@@H]2C[C@@H]1/C(=C\OC)C(=O)N(C)CCC(=O)OC ; CC[C@@H]1CN2CCc3c([nH]c4cccc(OC)c34)[C@@H]2C[C@@H]1/C(=C\OC)C(=O)N(C)c1ccccc1 ; CC[C@@H]1CN2CCc3c([nH]c4cccc(OC)c34)[C@@H]2C[C@@H]1/C(=C\OC)C(=O)N(C)c1ccccc1F ; CC[C@@H]1CN2CCc3c([nH]c4cccc(OC)c34)[C@@H]2C[C@@H]1/C(=C\OC)C(=O)N(C)c1ccccc1OC ; CC[C@@H]1CN2CCc3c([nH]c4cccc(OC)c34)[C@@H]2C[C@@H]1/C(=C\OC)C(=O)N(C)Cc1ccccc1]

or pharmaceutically acceptable salt thereof.

In some embodiments, the compound of Formula (I) is of formula:

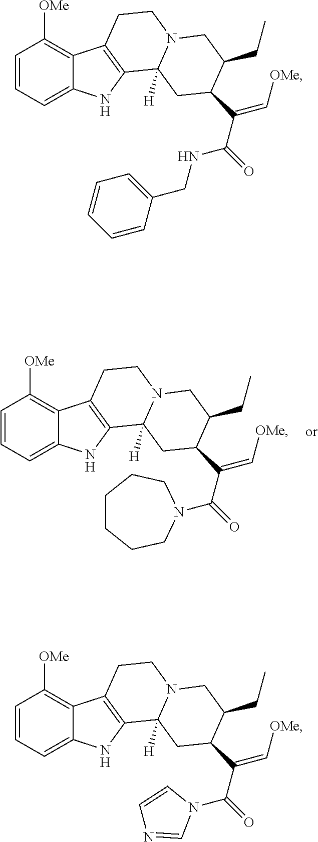 [SMILES: CC[C@@H]1CN2CCc3c([nH]c4cccc(OC)c34)[C@@H]2C[C@@H]1/C(=C\OC)C(=O)NCc1ccccc1 ; CC[C@@H]1CN2CCc3c([nH]c4cccc(OC)c34)[C@@H]2C[C@@H]1/C(=C\OC)C(=O)N1CCCCCC1 ; CC[C@@H]1CN2CCc3c([nH]c4cccc(OC)c34)[C@@H]2C[C@@H]1/C(=C\OC)C(=O)n1ccnc1]

or pharmaceutically acceptable salt thereof.

In some embodiments, the compound of Formula (I) is of Formula (I-B):

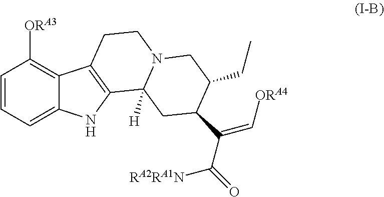

or pharmaceutically acceptable salt thereof.

In further embodiments, the compound of Formula (I) is of formula:

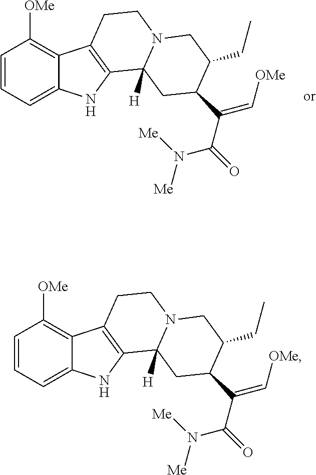 [SMILES: CC[C@H]1CN2CCc3c([nH]c4cccc(OC)c34)[C@H]2C[C@@H]1/C(=C\OC)C(=O)N(C)C ; CC[C@H]1CN2CCc3c([nH]c4cccc(OC)c34)[C@H]2C[C@@H]1/C(=C\OC)C(=O)N(C)C]

or pharmaceutically acceptable salt thereof.

Provided herein are compounds of Formula (II):

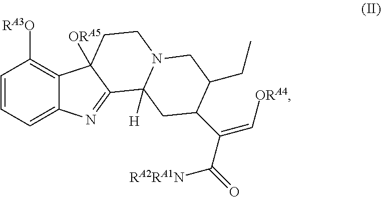

or pharmaceutically acceptable salt thereof, wherein:
R^{A1} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
   or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, or an oxygen protecting group;
R^{A4} is hydrogen, substituted or unsubstituted C₁₋₁₂ alkyl, or an oxygen protecting group; and
R^{A5} is hydrogen, substituted or unsubstituted C₁₋₁₂ alkyl, or an oxygen protecting group.

In some embodiments, the compound of Formula (II) is of Formula (II-A):

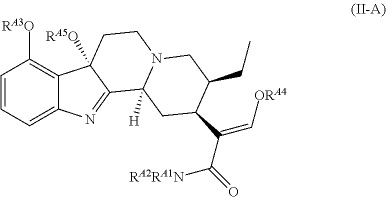

or pharmaceutically acceptable salt thereof.

In some embodiments, the compound of Formula (II) is of the formula:

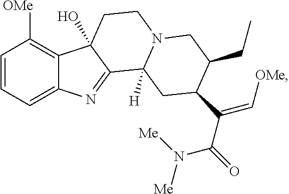 [SMILES: CC[C@@H]1CN2CC[C@@]3(O)C(=Nc4cccc(OC)c43)[C@@H]2C[C@@H]1/C(=C\OC)C(=O)N(C)C]

or pharmaceutically acceptable salt thereof.

In some embodiments, R^{A3} is hydrogen, substituted or unsubstituted C₁₋₁₂ alkyl, or an oxygen protecting group. In certain embodiments, R^{A3} is C₁₋₆ alkyl. In some embodiments, R^{A3} is methyl.

In certain embodiments, R^{A4} is hydrogen, substituted or unsubstituted C₁₋₁₂ alkyl, or an oxygen protecting group. In some embodiments, R^{A4} is C₁₋₆ alkyl. In certain embodiments, R^{A4} is methyl.

In some embodiments, R^{A1} is substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group; and R^{A2} is substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group.

In some embodiments, R^{A1} is hydrogen, substituted or unsubstituted C₁₋₁₂ alkyl, substituted or unsubstituted 3- to 13-membered carbocyclyl, substituted or unsubstituted 3-to 13-membered heterocyclyl, substituted or unsubstituted 6- to 12-membered aryl ring, substituted or unsubstituted 5- to 14-membered heteroaryl ring, or a nitrogen protecting group. In some embodiments, R^{A1} is hydrogen or unsubstituted C₁₋₆ alkyl. In certain embodiments, R^{A1} is hydrogen, methyl, ethyl, n-propyl, or isopropyl. In some embodiments, R^{A1} is C₁₋₆ alkyl substituted with one or more groups selected from —COOMe, —COOEt, fluoro, chloro, bromo, iodo, —NH₂, —NMe₂, —NEt₂, —OMe, —OEt, and phenyl. In some embodiments, R^{A1} is cyclopentyl or cyclohexyl. In certain embodiments, R^{A1} is unsubstituted phenyl. In certain embodiments, R^{A1} is phenyl substituted with one or more groups selected from —COOMe, —COOEt, fluoro, chloro, bromo, iodo, —NH₂, —NMe₂, —NEt₂, —OMe, —OEt, and phenyl.

In some embodiments, R^{A2} is hydrogen, substituted or unsubstituted C₁₋₁₂ alkyl, substituted or unsubstituted 3- to 13-membered carbocyclyl, substituted or unsubstituted 3-to 13-membered heterocyclyl, substituted or unsubstituted 6- to 12-membered aryl ring, substituted or unsubstituted 5- to 14-membered heteroaryl ring, or a nitrogen protecting group. In certain embodiments, R^{A2} is hydrogen or unsubstituted C₁₋₆ alkyl. In some embodiments, R^{A2} is hydrogen, methyl, ethyl, n-propyl, or isopropyl. In certain embodiments, R^{A2} is C₁₋₆ alkyl substituted with one or more groups selected from —COOMe, —COOEt, fluoro, chloro, bromo, iodo, —NH₂, —Nme₂, —NEt₂, —OMe, —OEt, and phenyl. In some embodiments, R^{A2} is cyclopentyl or cyclohexyl. In certain embodiments, R^{A2} is unsubstituted phenyl. In certain embodiments, R^{A2} is phenyl substituted with one or more groups selected from —COOMe, —COOEt, fluoro, chloro, bromo, iodo, —NH₂, —NMe₂, —NEt₂, —OMe, —OEt, and phenyl.

In certain embodiments, R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted. In some embodiments, R^{A1} and R^{A2} are joined with the intervening nitrogen atom to form a ring. In some embodiments, R^{A1} and R^{A2} are joined with the intervening nitrogen atom to form pyrrolinyl, piperidinyl, pyrrolyl, morpholinyl, piperazinyl, N-methyl piperazinyl, or thiomorpholinyl. In some embodiments, R^{A1} and R^{A2} are joined with the intervening nitrogen atom to form substituted or unsubstituted pyrrolinyl, substituted or unsubstituted piperidinyl, substituted or unsubstituted pyrrolyl, substituted or unsubstituted morpholinyl, substituted or unsubstituted piperazinyl, substituted or unsubstituted N-methyl piperazinyl, or substituted or unsubstituted thiomorpholinyl.

In some embodiments, R^{A5} is hydrogen, substituted or unsubstituted C₁₋₁₂ alkyl, or an oxygen protecting group. In certain embodiments, R^{A5} is methyl. In some embodiments, RAs is hydrogen.

### Compounds of Formula (III), (IV), (V), and (X)

Also provided herein are compounds of Formula (III):

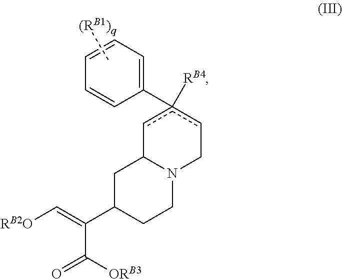

or a pharmaceutically acceptable salt thereof, wherein:
R^{B1} is hydrogen, halo, —OH, —O(C₁₋₆ alkyl), or C₁₋₆ haloalkyl;
R^{B2} is hydrogen, substituted or unsubstituted C₁₋₁₂ alkyl, or an oxygen protecting group;
R^{B3} is hydrogen, substituted or unsubstituted C₁₋₁₂ alkyl, or an oxygen protecting group;
R^{B4} is hydrogen, —OH, —OMe, —OEt, or absent, as valency permits;
q is 0, 1, 2, 3, 4, or 5; and
each instance of [symbol] is either a single or double bond.

In some embodiments, provided herein are compounds of Formula (III):

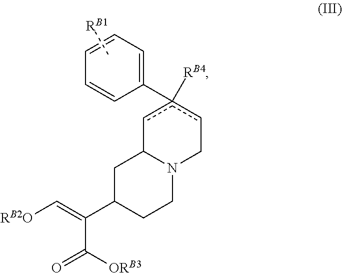

or a pharmaceutically acceptable salt thereof, wherein:
R^{B1} is hydrogen, halo, —OH, —O(C₁₋₆ alkyl), or C₁₋₆ haloalkyl;
R^{B2} is hydrogen, substituted or unsubstituted C₁₋₁₂ alkyl, or an oxygen protecting group;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

In some embodiments, provided herein are compounds of Formula (III):

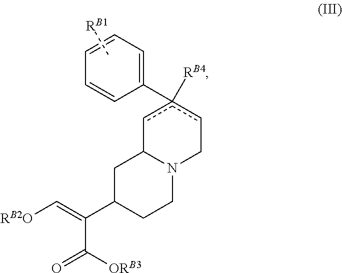

or a pharmaceutically acceptable salt thereof, wherein:
R^{B1} is hydrogen, fluoro, chloro, bromo, iodo, —OH, —OMe, or —OEt;
R^{B2} is hydrogen, substituted or unsubstituted C₁₋₁₂ alkyl, or an oxygen protecting group;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

In some embodiments, compound of Formula (III) is of formula:

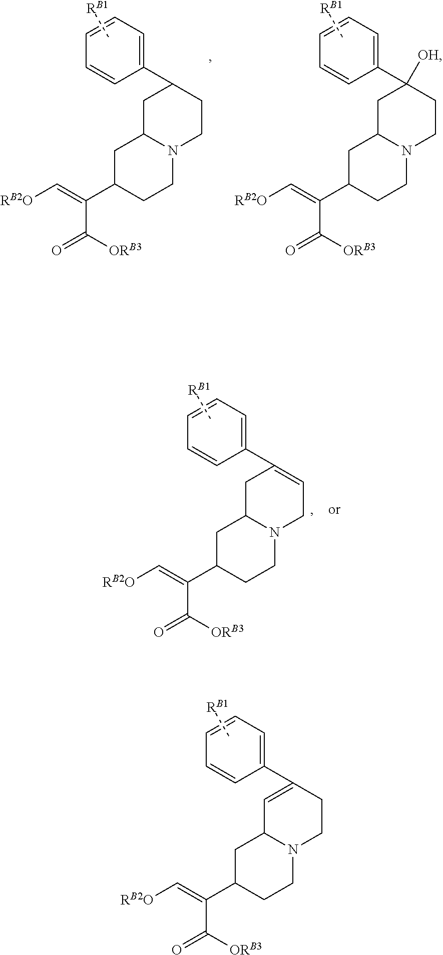

or a pharmaceutically acceptable salt thereof.

In some embodiments, the compound of Formula (III) is of formula:

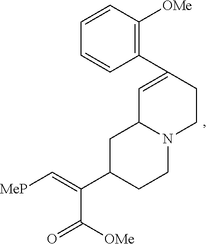

or a pharmaceutically acceptable salt thereof.

In some embodiments, the compound of Formula (III) is of formula:

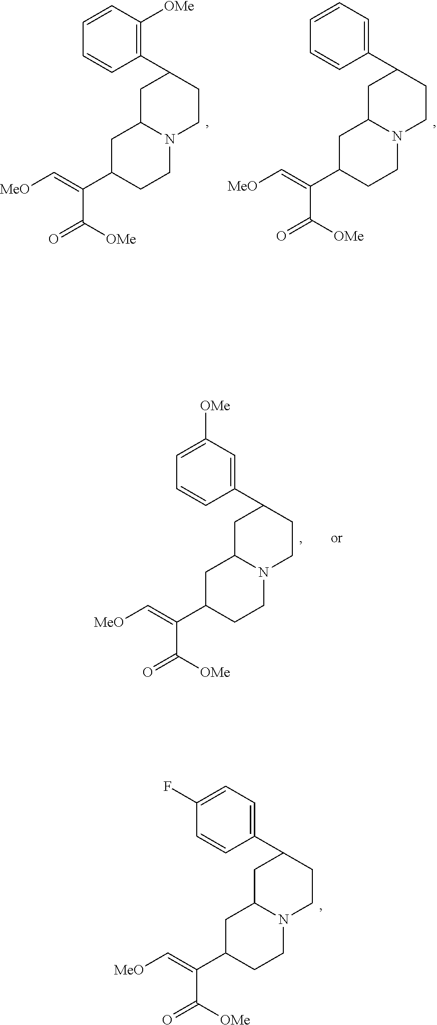 [SMILES: CO/C=C(\C(=O)OC)C1CCN2CCC(c3ccccc3OC)CC2C1 ; CO/C=C(\C(=O)OC)C1CCN2CCC(c3ccccc3)CC2C1 ; CO/C=C(\C(=O)OC)C1CCN2CCC(c3cccc(OC)c3)CC2C1 ; CO/C=C(\C(=O)OC)C1CCN2CCC(c3ccc(F)cc3)CC2C1]

or a pharmaceutically acceptable salt thereof.

Also provided herein are compounds of Formula (IV):

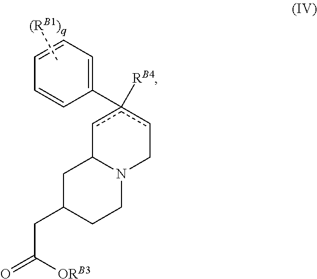

or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits;
q is 0, 1, 2, 3, 4, or 5; and
each instance [symbol] of is either a single or double bond.

In some embodiments, provided herein are compounds of Formula (IV):

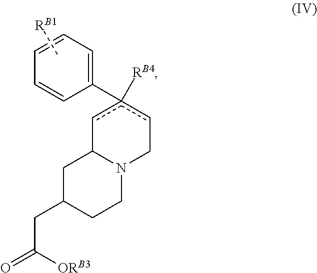

or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and each instance of [symbol] is either a single or double bond.

In some embodiments, provided herein are compounds of Formula (IV):

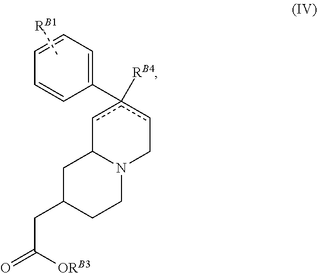

or a pharmaceutically acceptable salt thereof, wherein:
R^{B1} is hydrogen, fluoro, chloro, bromo, iodo, —OH, —OMe, or —OEt;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

In some embodiments, the compound of Formula (IV) is of formula:

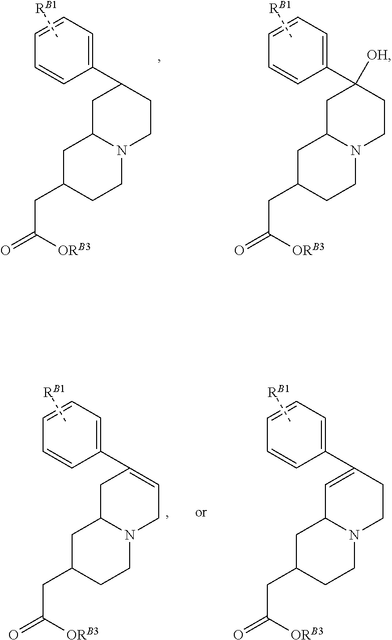

or a pharmaceutically acceptable salt thereof.

In some embodiments, the compound of Formula (IV) is of formula:

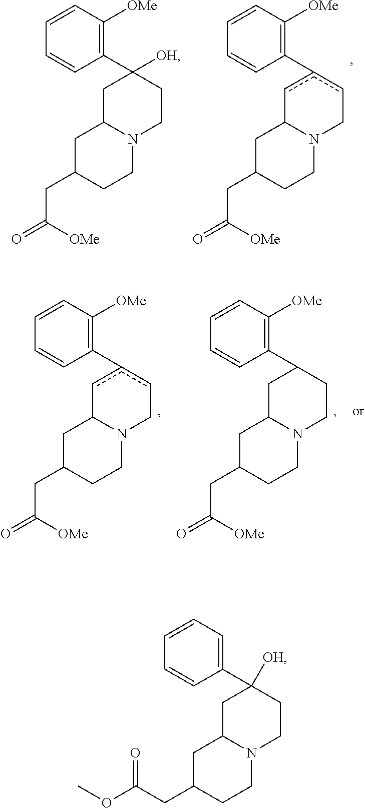

or a pharmaceutically acceptable salt thereof.

In some embodiments, the compound of Formula (IV) is of formula:

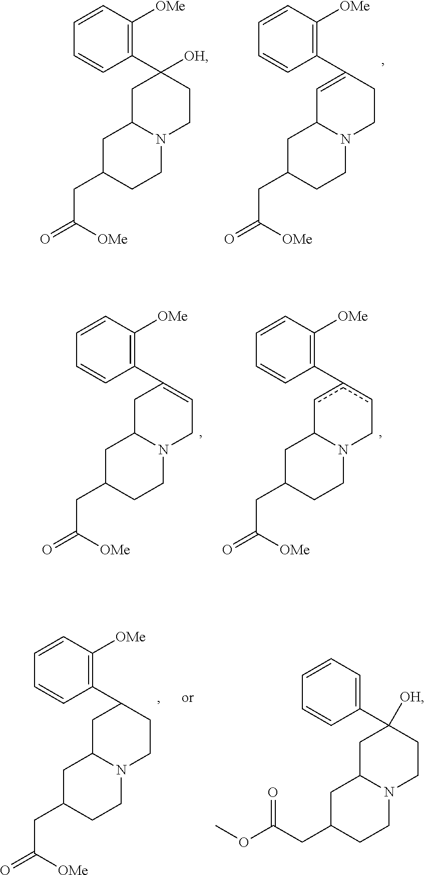

or a pharmaceutically acceptable salt thereof.

In some embodiments, the compound of Formula (IV) is of formula:

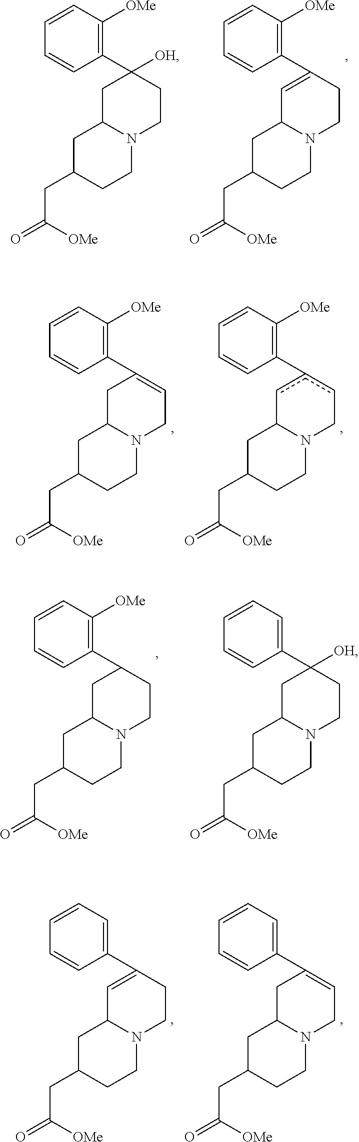

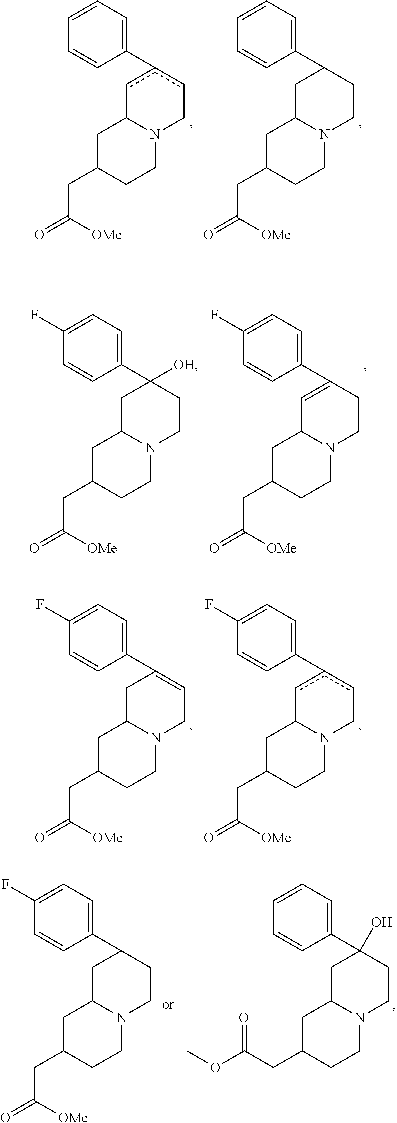

or a pharmaceutically acceptable salt thereof.

Also provided herein are compounds of Formula (V):

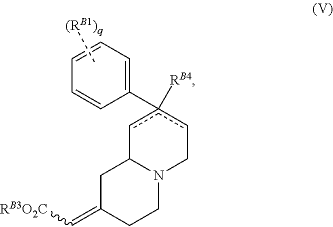

or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits;
q is 0, 1, 2, 3, 4, or 5; and
each instance of [symbol] is either a single or double bond.

In some embodiments, provided herein are compounds of Formula (V):

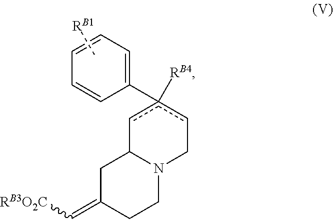

or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

In some embodiments, provided herein are compounds of Formula (V):

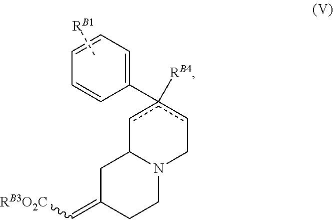

or a pharmaceutically acceptable salt thereof, wherein:
R^{B1} is hydrogen, fluoro, chloro, bromo, iodo, —OH, —OMe, or —OEt;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

In some embodiments, the compound of Formula (V) is of formula:

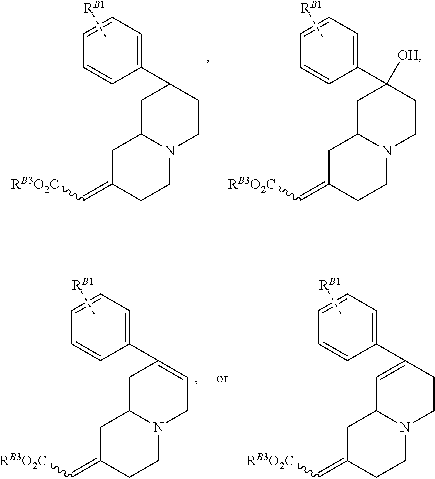

or a pharmaceutically acceptable salt thereof.

In some embodiments, the compound of Formula (V) is of formula:

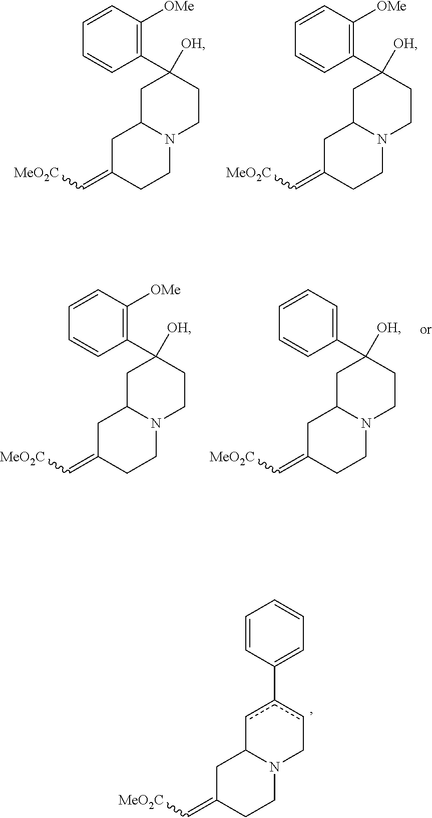

or a pharmaceutically acceptable salt thereof.

In some embodiments, the compound of Formula (V) is of formula:

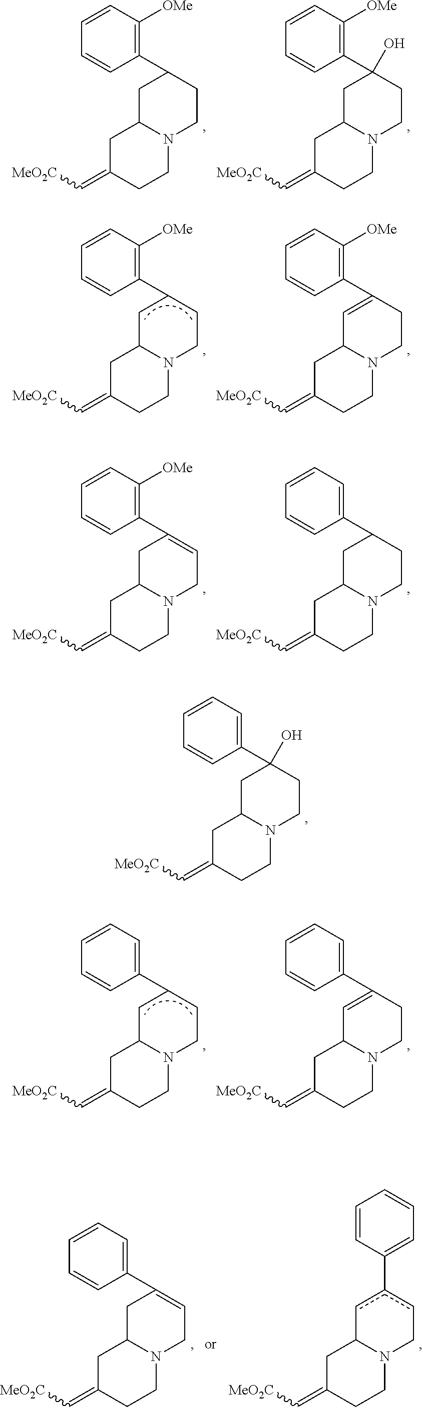

or a pharmaceutically acceptable salt thereof.
In some embodiments, the compound of Formula (V) is of formula:

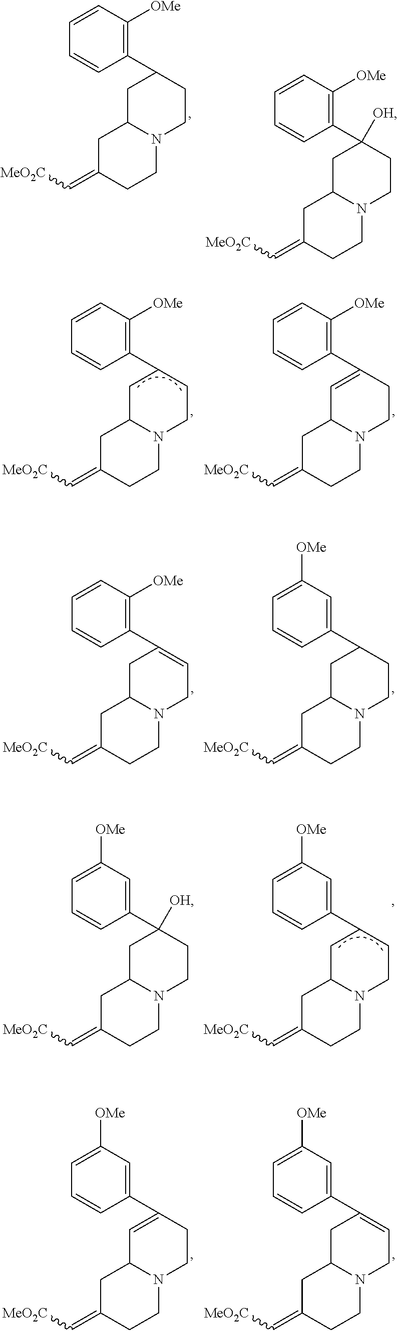

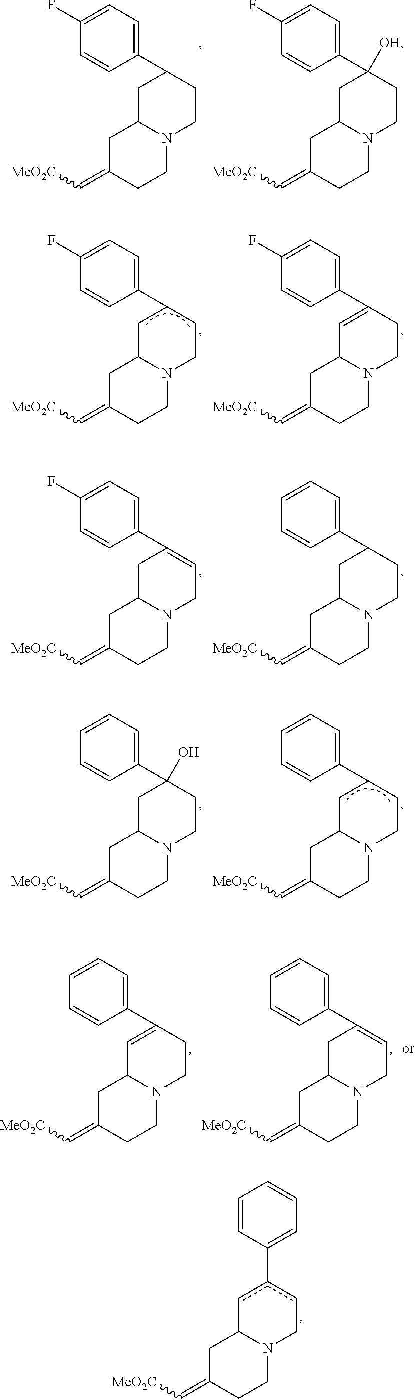

or a pharmaceutically acceptable salt thereof.

In some embodiments, the alkene bearing —CO₂Me is in the cis formation. In some embodiments, the alkene bearing —CO₂Me is in the trans formation.

In some embodiments, the compound of Formula (V) is of formula:

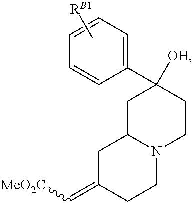

or a pharmaceutically acceptable salt thereof, wherein R^{B1} is 2-OMe, H, 4-fluoro, or 3-OMe.
In some embodiments, R^{B1} is 3-OMe and the alkene bearing —CO₂Me is in the cis formation.
In some embodiments, R^{B1} is 3-OMe and the alkene bearing —CO₂Me is in the trans formation.

Also provided herein are compounds of Formula (X):

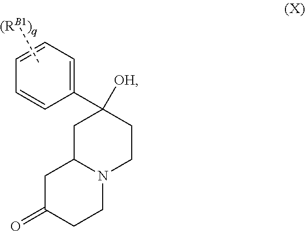

or a pharmaceutically acceptable salt thereof, wherein:
R^{B1} is hydrogen, halo, —OH, —O(C₁₋₆ alkyl), or C₁₋₆ haloalkyl; and
q is 0, 1, 2, 3, 4, or 5.

In some embodiments, provided herein are compounds of Formula (X):

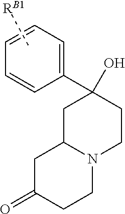

or a pharmaceutically acceptable salt thereof, wherein:
R^{B1} is hydrogen, halo, —OH, —O(C₁₋₆ alkyl), or C₁₋₆ haloalkyl.

In some embodiments, provided herein are compounds of Formula (X):

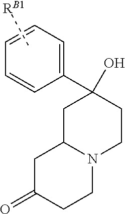

or a pharmaceutically acceptable salt thereof, wherein:
R^{B1} is hydrogen, fluoro, chloro, bromo, iodo, —OH, —OMe, or —OEt.

In certain embodiments, the compound of Formula (X) is of formula:

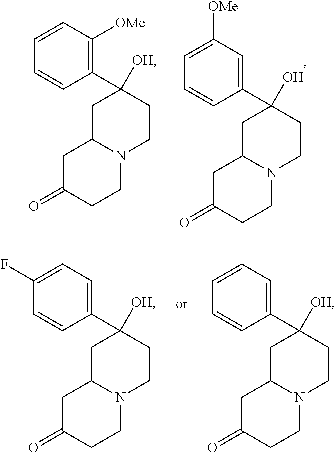

or a pharmaceutically acceptable salt thereof.

In some embodiments, R^{B1} is hydrogen, halo, —OH, —O(C₁₋₆ alkyl), or optionally substituted C₁₋₆ alkyl. In some embodiments, R^{B1} is hydrogen, halo, —OH, —O(C₁₋₆ alkyl), or C₁₋₆ haloalkyl. In some embodiments, R^{B1} is hydrogen, fluoro, chloro, bromo, iodo, —OH, —OMe, or —OEt. In certain embodiments, R^{B1} is hydrogen, fluoro, or —OMe. In some embodiments, R^{B1} is hydrogen. In some embodiments, R^{B1} is —OMe. In certain embodiments, R^{B1} is ortho —OMe. In certain embodiments, R^{B1} is meta —OMe. In certain embodiments, R^{B1} is para —OMe. In some embodiments, R^{B1} is fluoro. In certain embodiments, R^{B1} is ortho fluoro. In certain embodiments, R^{B1} is meta fluoro. In certain embodiments, R^{B1} is para fluoro.

In certain embodiments, R^{B2} is hydrogen, substituted or unsubstituted C₁₋₁₂ alkyl, or an oxygen protecting group. In some embodiments, R^{B3} is hydrogen or C₁₋₄ alkyl. In certain embodiments, R^{B3} is methyl.

In some embodiments, R^{B3} is hydrogen, substituted or unsubstituted C₁₋₁₂ alkyl, or an oxygen protecting group. In some embodiments, R^{B3} is hydrogen or C₁₋₄ alkyl. In certain embodiments, R^{B3} is methyl.

In certain embodiments, R^{B4} is hydrogen, —OH, —OMe, —OEt, or absent, as valency permits. In some embodiments, R^{B4} is hydrogen or —OH. In certain embodiments, R^{B4} is hydrogen. In some embodiments, R^{B4} is —OH. In some embodiments, R^{B4} is absent.

In some embodiments, q is 0, 1, 2, 3, 4, or 5. In some embodiments, q is 0, 1, or 2. In some embodiments, q is 0. In some embodiments, q is 1. In certain embodiments, q is 2. In some embodiments, q is 3. In certain embodiments, q is 4. In some embodiments, q is 5.

In some embodiments, each instance of [symbol] is either a single or double bond. In some embodiments, [symbol] is a single bond. In certain embodiments, [symbol] is a double bond. In some embodiments, one instance of [symbol] is a single bond and one instance of [symbol] is a double bond.

### Compounds of Formula (VI) and (VII)

Provided herein are compounds of Formula (VI):

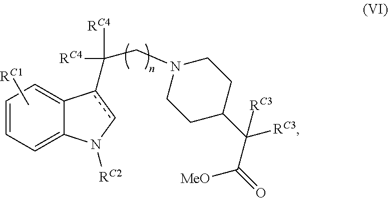

or pharmaceutically acceptable salt thereof, wherein:
R^{C1} is hydrogen, —OH, or —OMe;
R^{C2} is hydrogen, unsubstituted C₁₋₆ alkyl, or C₁₋₆ alkyl substituted with one or more instances of —OH, —OMe, fluoro, indolyl;
each instance of R^{C3} is hydrogen or both instances of R^{3C} are taken together to form:

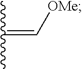 [SMILES: *=COC]

each instance of R^{C4} is hydrogen or both instances of R^{C4} are taken together to form: ═O; and
n is 0, 1, or 2.

In some embodiments, the compound of Formula (VI) is of formula:

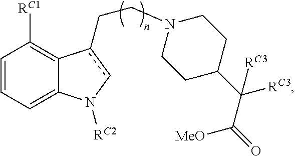

or pharmaceutically acceptable salt thereof.

In some embodiments, the compound of Formula (VI) is of formula:

or pharmaceutically acceptable salt thereof.

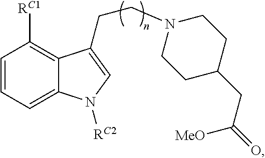

In some embodiments, the compound of Formula (VI) is of formula:

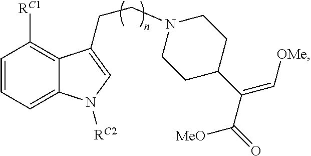

or pharmaceutically acceptable salt thereof.

In certain embodiments, the compound of Formula (VI) is of formula:

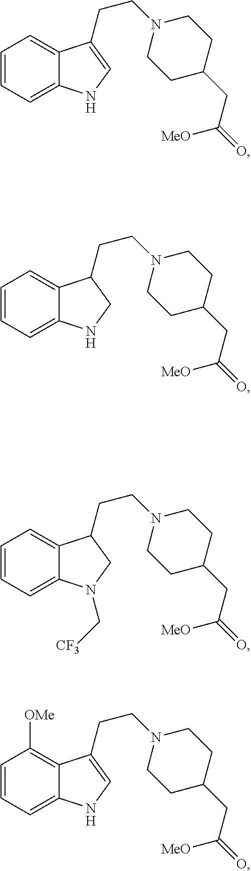 [SMILES: COC(=O)CC1CCN(CCc2c[nH]c3ccccc23)CC1 ; COC(=O)CC1CCN(CCC2CNc3ccccc32)CC1 ; COC(=O)CC1CCN(CCC2CN(CC(F)(F)F)c3ccccc32)CC1 ; COC(=O)CC1CCN(CCc2c[nH]c3cccc(OC)c23)CC1]

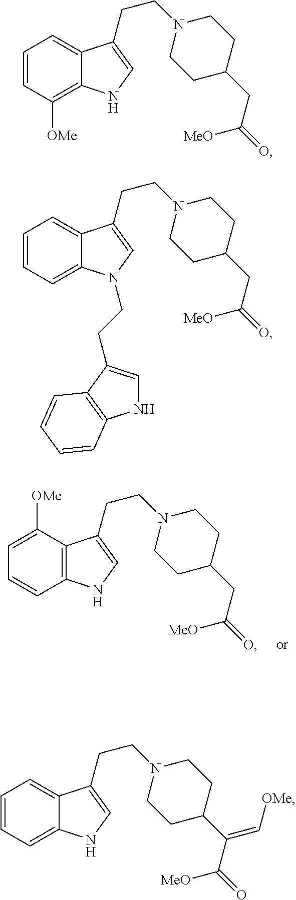 [SMILES: COC(=O)CC1CCN(CCc2c[nH]c3c(OC)cccc23)CC1 ; COC(=O)CC1CCN(CCc2cn(CCc3c[nH]c4ccccc34)c3ccccc23)CC1 ; COC(=O)CC1CCN(CCc2c[nH]c3cccc(OC)c23)CC1 ; CO/C=C(/C(=O)OC)C1CCN(CCc2c[nH]c3ccccc23)CC1]

or pharmaceutically acceptable salt thereof.

Also provided herein are compounds of Formula (VII):

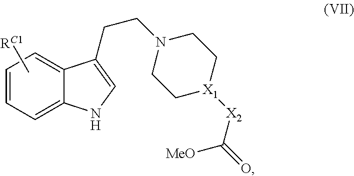

or pharmaceutically acceptable salt thereof, wherein:
R^{C1} is hydrogen, —OH, or —OMe;
X₁ is CH or N; and
X₂ is NH, CH₂, or CH—(CH₂)—OMe.

In some embodiments, the compound of Formula (VII) is of formula:

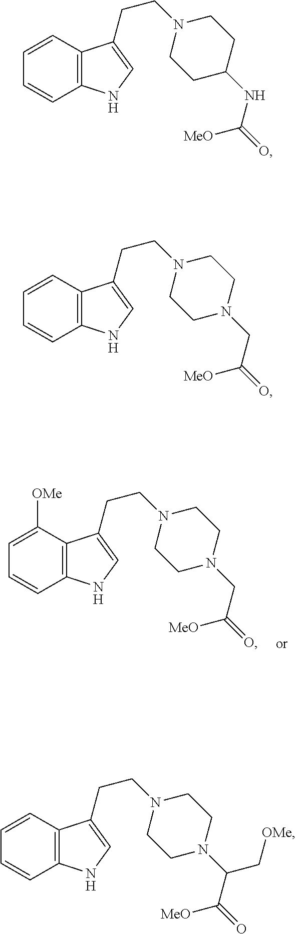 [SMILES: COC(=O)NC1CCN(CCc2c[nH]c3ccccc23)CC1 ; COC(=O)CN1CCN(CCc2c[nH]c3ccccc23)CC1 ; COC(=O)CN1CCN(CCc2c[nH]c3cccc(OC)c23)CC1 ; COCC(C(=O)OC)N1CCN(CCc2c[nH]c3ccccc23)CC1]

or pharmaceutically acceptable salt thereof.

In some embodiments, R^{C1} is hydrogen, —OH, or —OMe. In certain embodiments, R^{C1} is hydrogen or —OMe. In some embodiments, R^{C1} is —OMe.

In certain embodiments, R^{C2} is hydrogen, unsubstituted C₁₋₆ alkyl, or C₁₋₆ alkyl substituted with one or more instances of —OH, —OMe, fluoro, indolyl. In some embodiments, R^{C2} is hydrogen or C₁₋₆ alkyl substituted with -fluoro or indolyl. In some embodiments, R^{C2} is hydrogen or C₁₋₆ alkyl substituted with -one or more instances of fluoro or indolyl. In some embodiments, R^{C2} is hydrogen, —CH₂CF₃, —CF₃, or C₁₋₆ alkyl substituted with indolyl.

In some embodiments, each instance of RC³ is hydrogen or both instances of R^{3C} are taken together to form:

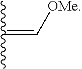 [SMILES: *=COC]

In certain embodiments, each instance of R^{C4} is hydrogen or both instances of R^{C4} are taken together to form: ═O. In certain embodiments, each instance of R^{C4} is hydrogen. In certain embodiments, each instance of R^{C4} are taken together to form: ═O.

In some embodiments, n is 0, 1, or 2. In certain embodiments, n is 0. In some embodiments, n is 1. In some embodiments, n is 2.

In certain embodiments, X₁ is CH or N. In certain embodiments, X₁ is CH. In certain embodiments, X₁ is N.

In some embodiments, X₂ is NH, CH₂, or CH—(CH₂)—OMe. In some embodiments, X₂ is NH. In some embodiments, X₂ is CH₂. In some embodiments, X₂ is CH—(CH₂)—OMe.

In some embodiments, X₁ is N and X₂ is CH₂. In certain embodiments, X₁ is CH and X₂ is NH. In some embodiments, X₁ is CH and X₂ is CH—(CH₂)—OMe.

### Compounds of Formula (VIII)

Also provided herein are compounds of Formula (VIII):

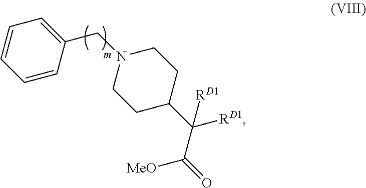

or pharmaceutically acceptable salt thereof, wherein:
m is 1, 2, or 3; and
each instance of R^{D1} is hydrogen or both instances of R^{D1} are taken together to form:

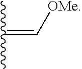 [SMILES: *=COC]

In some embodiments, each instance of R^{D1} is hydrogen. In some embodiments, both instances of R^{D1} are taken together to form:

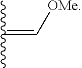 [SMILES: *=COC]

In some embodiments, m is 1, 2, or 3. In some embodiments, m is 1. In certain embodiments, m is 2. In some embodiments, m is 3

### Compounds of Formula (IX)

Also provided herein are compounds of Formula (IX):

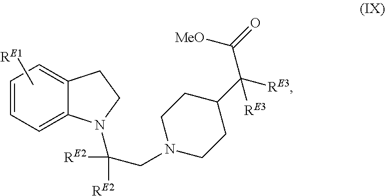

or pharmaceutically acceptable salt thereof, wherein:
R^{E1} is hydrogen, —OH, or —OMe;
each instance of R^{E2} is hydrogen or both instances of R^{E2} are taken together to form: ═O; and
each instance of R^{E3} is hydrogen or both instances of R^{E3} are taken together to form:

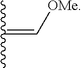 [SMILES: *=COC]

In certain embodiments, the compound of Formula (IX) is of formula:

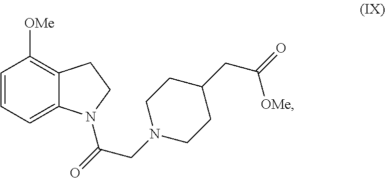 [SMILES: COC(=O)CC1CCN(CC(=O)N2CCc3c(OC)cccc32)CC1]

or pharmaceutically acceptable salt thereof.

In some embodiments, R^{E1} is hydrogen, —OH, or —OMe. In certain embodiments, R^{E1} is hydrogen or —OMe. In some embodiments, R^{E1} is —OMe.

In certain embodiments, each instance of ^{E2} is hydrogen or both instances of R^{E2} are taken together to form: ═O. In certain embodiments, each instance of R^{E2} is hydrogen. In certain embodiments, each instance of R^{E2} are taken together to form: ═O.

In some embodiments, each instance of R^{E3} is hydrogen. In some embodiments, both instances of R^{E3} are taken together to form:

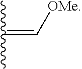 [SMILES: *=COC]

### Compositions, Administration, and Kits

The present disclosure provides pharmaceutical compositions comprising a compound of Formula (I′), (I), (II), (III), (IV), (V), (VI), (VII), (VIII), or (IX), or a pharmaceutically acceptable salt thereof, and optionally a pharmaceutically acceptable excipient. In certain embodiments, the pharmaceutical composition described herein comprises a compound of Formula (I′), (I), (II), (III), (IV), (V), (VI), (VII), (VIII), or (IX), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, painful condition, psychiatric disorder, or metabolic disorder) in a subject in need thereof.

In certain embodiments, the disorder is a psychiatric disorder. A psychiatric disorder includes, for example, depression, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder.

In certain embodiments, the disorder is a painful condition or symptoms associated with a painful condition. In certain embodiments, the painful condition is induced (i.e., caused) by nociceptive pain. In certain embodiments, the nociceptive pain is the caused by a chemical, mechanical, or thermal stimulus. In certain embodiments, the painful condition is pain associated with withdrawal from an addiction.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

In certain embodiments, the effective amount is an amount effective for inhibiting the activity of a protein (e.g., receptor (e.g., an opioid receptor and/or an adrenergic receptor)) by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of an opioid receptor and/or an adrenergic receptor by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of an opioid receptor and/or an adrenergic receptor by a range between a percentage described in this paragraph and another percentage described in this paragraph, inclusive.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymers and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional components other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymers and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein (e.g., a receptor (e.g., an opioid receptor and/or an adrenergic receptor)) in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

The present disclosure also provides a kit comprising: a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)); and instructions for administering the compound, or pharmaceutically acceptable salt thereof, or composition to a subject.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein (e.g., receptor (e.g., opioid receptor and/or adrenergic receptor)) in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological disease, opioid receptor mediated disorder, painful condition, or psychiatric disorder) in a subject in need thereof. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein in a subject or cell. A kit described herein may include one or more additional pharmaceutical agents described herein as a separate composition.

### Methods

Provided herein are methods utilizing and uses of the compounds disclosed herein (e.g., compounds of Formula (I′), (I), (II), (III), (IV), (V), (VI), (VII), (VIII), (IX)) or pharmaceutically acceptable salt thereof, or a composition provided herein (e.g., a composition comprising a compound of Formula (I′), (I), (II), (III), (IV), (V), (VI), (VII), (VIII), (IX)).

In an additional aspect, the present disclosure provides methods of treating or preventing substance intake by a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In another aspect, provided herein are methods of treating or preventing substance use disorder in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In one aspect, the present disclosure provides methods of treating the symptoms of substance use disorder in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In an additional aspect, the present disclosure provides methods of treating or preventing substance addiction in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In another aspects, provided herein are methods of treating the symptoms of substance addiction in a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In one aspect, the present disclosure provides methods of treating or preventing neurotoxic effects resulting from substance use disorder, substance addiction, and/or substance intake by a subject comprising administering to the subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In a further aspect, provided herein are methods of treating or preventing a disease or disorder associated with one or more opioid receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In a further aspect, provided herein are methods of treating or preventing a disease or disorder associated with one or more adrenergic receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

Another object of the present disclosure is the use of a compound as described herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) in the manufacture of a medicament for use in the treatment of a disorder or disease described herein. Another object of the present disclosure is the use of a compound as described herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) for use in the treatment of a disorder or disease described herein. Another object of the present disclosure is the use of a compound as described herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) in the manufacture of a veterinary composition for use in the treatment or prevention of a disorder or disease in veterinary applications.

In some embodiments, provided herein are methods of treating or preventing a disease or disorder comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In some embodiments, the disease or disorder is associated with opioid receptors and/or adrenergic receptors.

In some embodiments, provided herein are methods of treating or preventing a disease or disorder associated with one or more opioid receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In some embodiments, provided herein are methods of treating or preventing a disease or disorder associated with one or more adrenergic receptors comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In some embodiments, provided herein are methods of treating or preventing substance withdrawal comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In some embodiments, provided herein are methods of treating or preventing symptoms associated with substance withdrawal comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In some embodiments, provided herein are methods of treating or preventing substance dependence comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In another aspect, provided herein is a method of treating a subject suffering from a painful condition or symptoms thereof, the method comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)). In certain embodiments, the painful condition is induced by nociceptive pain. In some embodiments, the nociceptive pain is the caused by a chemical, mechanical, or thermal stimulus. In certain embodiments, the painful condition is neuropathic pain.

In a further aspect, provided herein is a method of treating a subject in need of an analgesic, the method comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)).

In one embodiment, the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or preventing substance addiction in the subject.

In one embodiment, the method the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or preventing substance use in the subject.

In one embodiment, the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or preventing substance seeking behavior in the subject. In some embodiments, the substance seeking behavior is stress induced. In some embodiments, the substance seeking behavior is related to a relapse. In some embodiments, the substance seeking behavior is stress induced and related to a relapse.

In one embodiment, the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or prevention of substance use.

The compounds of Formula (I′), (I), (II), (III), (IV), (V), (VI), (VII), (VIII), and (IX), or pharmaceutically acceptable salt thereof, and the compositions provided herein (e.g., a composition comprising a compound of Formula (I′), (I), (II), (III), (IV), (V), (VI), (VII), (VIII), (IX)) are useful in treating abuse of substances, inhibiting the abuse of substances, and/or preventing the abuse of substances.

In one embodiment, the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or prevention of depression.

In one embodiment, the method comprises administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject for treating, inhibiting, and/or prevention of anxiety.

In another aspect, provided herein is a method for reducing or preventing nociception (e.g., promoting antinociception), wherein the method comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject.

In another aspect, provided herein is a method of treating a subject with a disease, disorder, or symptoms thereof, wherein the method comprising administering to a subject a therapeutically effective amount of a compound provided herein (e.g., a compound of Formula (I′), (I), (II), (III), (IV), (V), (VI), (VII), (VIII), (IX)), or pharmaceutically acceptable salt thereof, or a composition provided herein (e.g., a composition comprising a compound of Formula (I′), (I), (II), (III), (IV), (V), (VI), (VII), (VIII), (IX)) to a subject.

In some embodiments, the adrenergic receptor is an alpha-2 receptor. In certain embodiments, the adrenergic receptor is α₂A and/or α₂C receptor.

In some embodiments, the disease or disorder is a psychiatric disorder, a painful condition, or a neurological disorder. In some embodiments, the disease or disorder is depression, mood disorder, nociceptive pain, neuropathic pain, or pain associated with withdrawal symptoms from substance addiction.

In some embodiments, the substance is an opioid. In certain embodiments, the substance is a prescription opioid. In some embodiments, the substance is codeine, fentanyl, hydromorphone, meperidine, methadone, morphine, oxycodone, or oxymorphone. In certain embodiments, the substance is an illicit opioid. In some embodiments, the substance is heroin.

In some embodiments, the opioid receptor is a kappa opioid receptor, delta opioid receptor, and/or a mu opioid receptor. In some embodiments, the opioid receptor is a kappa opioid receptor. In some embodiments, the opioid receptor is a delta opioid receptor. In some embodiments, the opioid receptor is a mu opioid receptor.

In certain embodiments, the disorder is a psychiatric disorder. A psychiatric disorder includes, for example, depression, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder.

In certain embodiments, the disorder is a painful condition or symptoms associated with a painful condition. In certain embodiments, the painful condition is induced (i.e., caused) by nociceptive pain. In certain embodiments, the nociceptive pain is the caused by a chemical, mechanical, or thermal stimulus. In certain embodiments, the painful condition is pain associated with withdrawal from an addiction.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the disorder is a neurological disorder. In certain embodiments, the neurological disorder is addiction. In certain embodiments, the addiction is a substance addiction. In certain embodiments, the addiction is an opioid addiction.

In certain embodiments, the disorder is a psychiatric disorder. A psychiatric disorder includes, for example, depression, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder. In certain embodiments, the substance-related disorder is a substance abuse. In some embodiments, the psychiatric disorder is depression.

In certain embodiments, the disorder is a psychiatric disorder. A psychiatric disorder includes, for example, anxiety disorders, mood disorders, personality disorders, psychotic disorders, and substance-related disorders, among others. In certain embodiments, the psychiatric disorder is a mood disorder. In certain embodiments, the psychiatric disorder is an anxiety disorder. In certain embodiments, the psychiatric disorder is a substance-related disorder. In certain embodiments, the substance-related disorder is a substance abuse. In certain embodiments, the substance abuse is opioid abuse.

In certain embodiments, the disorder is a painful condition or symptoms associated with a painful condition. In certain embodiments, the painful condition is induced (i.e., caused) by nociceptive pain. In certain embodiments, the nociceptive pain is the caused by a chemical, mechanical, or thermal stimulus. In certain embodiments, the painful condition is pain associated with withdrawal from an addiction. In certain embodiments, the addiction is an alcohol addiction. In certain embodiments, the addiction is a substance addiction. In certain embodiments, the substance addiction is an opioid addiction.

In certain embodiments, the methods of the disclosure include administering to a subject an effective amount of a compound described herein in combination with another pharmaceutically active compound. Pharmaceutically active compounds that may be used can be found in Harrison's Principles of Internal Medicine, Nineteenth Edition, Eds. T. R. Harrison et al. McGraw-Hill N.Y., NY; and the Physicians Desk Reference 71st Edition 2017, Oradell New Jersey, Medical Economics Co., the complete contents of which are expressly incorporated herein by reference. The compound of the disclosure and the pharmaceutically active compound may be administered to the subject in the same pharmaceutical composition or in different pharmaceutical compositions (at the same time or at different times).

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired.

### EXAMPLES

In order that the present disclosure may be more fully understood, the following examples are set forth. The synthetic and biological examples described in this Application are offered to illustrate the compounds, pharmaceutical compositions, methods, and uses provided herein and are not to be construed in any way as limiting their scope.

### Compounds of Formulas (I′), (I) and (II)

### Radioligand Binding Data:

Binding affinity of mitragynine amides were studied for both opioid (hMOR, hKOR, hDOR) and adrenergic receptor (alpha-2A and 2C) using radioligand displacement assay. Most of the mitragynine amides showed greater binding affinities at hMOR compared to hKOR and hDOR (Kᵢ>5-10 μM). Interesting trends towards the opioid binding affinity is observed when the synthesized series of 15 derivatives of mitragynine amide are categorized in two different classes: mono-alkylated amides (SUSM-25, SUSM-32, SUSM-27, SUSM-506, SUSM-519 and SUSM-528) and dialkylated amides (SUSM-501, SUSM-509, SUSM-507, SUSM-518, SUSM-520, SUSM-521, SUSM-522, SUSM-523 and SUSM-526). Most of the mono-alkylated amides where one of the N—H bond of amide is intact in that case poor opioid binding results was observed (Kᵢ values between 1 μM-7 μM). However, greater binding affinities was observed for di-alkylated amides (Kᵢ values between 67.8 nM-1.1 M). Among the tested di-alkylated amides SUSM-522 and SUSM-523 have the highest and selective affinity for hMOR with a Kᵢ value of 67.8 nM, and 81.9 nM, followed by SUSM-521 and SUSM-518 (Kᵢ=167 nM and 129 nM respectively). Heteroaromatic substituted amide SUSM-526 with pyrrole as amide substitution exhibited moderate affinity towards hMOR with Kᵢ=21. 6 μM and hKOR Kᵢ=57.9 μM. Next, functional activity of SUSM-501 was carried out at MOR (Eₘₐₓ=−4.05), suggesting the compound acts as partial antagonist. The compound SUSM-524 which is a 7-OH version of SUSM-501 exhibited 4-fold more potency than SUSM-501 at MOR with Kᵢ=110 nM.

TABLE A1
Summary of radioligand binding of mitragynine amides-data summary #1

| | | | KOR | DOR | Alpha2A | Alpha2C | MOR |
|---|---|---|---|---|---|---|---|
| | | MOR | Ki ± | Ki ± | Ki ± | Ki ± | Emax ± |
| | | Ki ± SEM | SEM | SEM | SEM | SEM | SEM (% |
| Structure | Code | (nM) | (nM) | (nM) | (nM) | (nM) | DAMGO) |
| 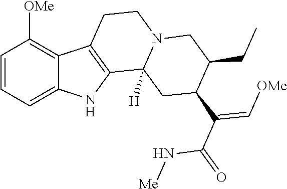 [SMILES: CC[C@@H]1CN2CCc3c([nH]c4cccc(OC)c34)[C@@H]2C[C@@H]1/C(=C\OC)C(=O)NC] | SUSM-25 | 4020 ± 728 | >10,000 | >10,000 | 7040 ± 1820 | NT | |
| 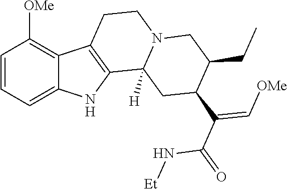 [SMILES: CCNC(=O)/C(=C/OC)[C@H]1C[C@H]2c3[nH]c4cccc(OC)c4c3CCN2C[C@H]1CC] | SUSM-32 | 4220 ± 752 | >10,000 | >10,000 | 4290 ± 1120 | NT | |
| 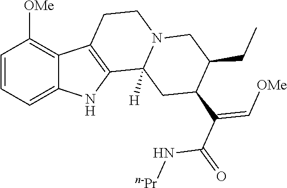 | SUSM-27 | 4560 ± 692 | >10,000 | >10,000 | 2730 ± 1260 | NT | |
| 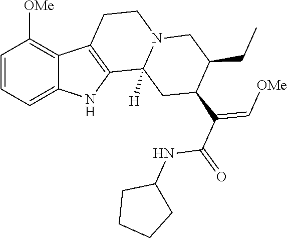 [SMILES: CC[C@@H]1CN2CCc3c([nH]c4cccc(OC)c34)[C@@H]2C[C@@H]1/C(=C\OC)C(=O)NC1CCCC1] | SUSM-506 | 1380 ± 176 | 5850 ± 657 | >10,000 | 8970 ± 572 | NT | |
| 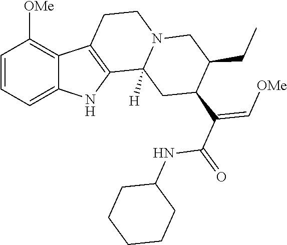 [SMILES: CC[C@@H]1CN2CCc3c([nH]c4cccc(OC)c34)[C@@H]2C[C@@H]1/C(=C\OC)C(=O)NC1CCCCC1] | SUSM-519 | 3590 ± 663 | 2790 ± 640 | >10,000 | NT | NT | |
| 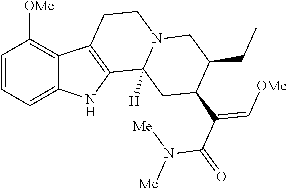 [SMILES: CC[C@@H]1CN2CCc3c([nH]c4cccc(OC)c34)[C@@H]2C[C@@H]1/C(=C\OC)C(=O)N(C)C] | SUSM-501 | 429 ± 75.7 | 3070 ± 569 | 6490 ± 3030 | 1810 ± 319 | 420 ± 113 | −4.05 ± 4.03 |
| 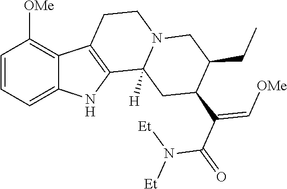 [SMILES: CC[C@@H]1CN2CCc3c([nH]c4cccc(OC)c34)[C@@H]2C[C@@H]1/C(=C\OC)C(=O)N(CC)CC] | SUSM-509 | 1130 ± 73.8 | >10,000 | >10,000 | 5200 ± 621 | NT | |
| 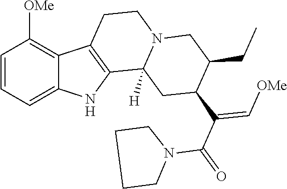 [SMILES: CC[C@@H]1CN2CCc3c([nH]c4cccc(OC)c34)[C@@H]2C[C@@H]1/C(=C\OC)C(=O)N1CCCC1] | SUSM-507 | 655 ± 76.6 | >10,000 | >10,000 | 9851 ± 1570 | NT | |
| 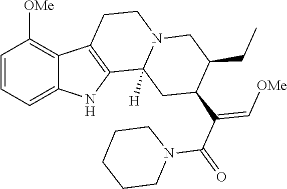 [SMILES: CC[C@@H]1CN2CCc3c([nH]c4cccc(OC)c34)[C@@H]2C[C@@H]1/C(=C\OC)C(=O)N1CCCCC1] | SUSM-518 | 129 ± 29.1 | 7460 ± 1420 | >10,000 | NT | NT | |
| 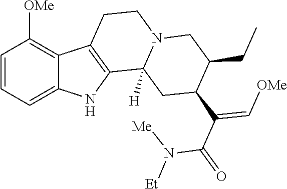 [SMILES: CC[C@@H]1CN2CCc3c([nH]c4cccc(OC)c34)[C@@H]2C[C@@H]1/C(=C\OC)C(=O)N(C)CC] | SUSM-520 | 459 ± 40.7 | 4780 ± 1140 | >10,000 | NT | NT | |
| 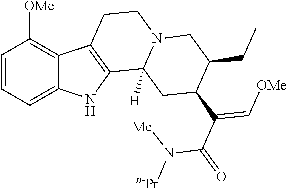 | SUSM-521 | 167 ± 2.15 | 2610 ± 588 | 5890 ± 105 | NT | NT | |
| 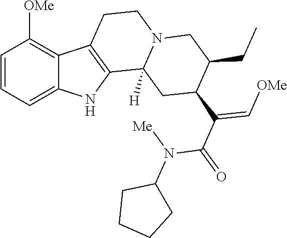 [SMILES: CC[C@@H]1CN2CCc3c([nH]c4cccc(OC)c34)[C@@H]2C[C@@H]1/C(=C\OC)C(=O)N(C)C1CCCC1] | SUSM-522 | 67.8 ± 7.02 | 4600 ± 132 | 9750 ± 452 | NT | NT | |
| 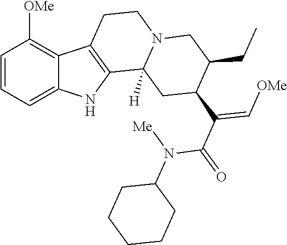 [SMILES: CC[C@@H]1CN2CCc3c([nH]c4cccc(OC)c34)[C@@H]2C[C@@H]1/C(=C\OC)C(=O)N(C)C1CCCCC1] | SUSM-523 | 81.9 ± 9 | 2640 ± 685 | 6490 ± 962 | NT | NT | |
| 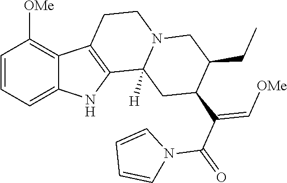 [SMILES: CC[C@@H]1CN2CCc3c([nH]c4cccc(OC)c34)[C@@H]2C[C@@H]1/C(=C\OC)C(=O)n1cccc1] | SUSM-526 | 2161 ± 941 | 5798 ± 1392 | NT | NT | NT | |
| 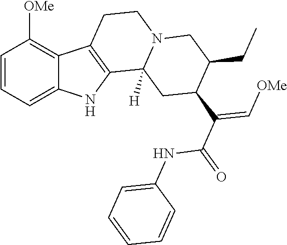 [SMILES: CC[C@@H]1CN2CCc3c([nH]c4cccc(OC)c34)[C@@H]2C[C@@H]1/C(=C\OC)C(=O)Nc1ccccc1] | SUSM-528 | 7772 ± 3622 | >10,000 | NT | 6584 ± 2316 | NT | |
| 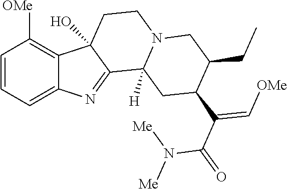 [SMILES: CC[C@@H]1CN2CC[C@@]3(O)C(=Nc4cccc(OC)c43)[C@@H]2C[C@@H]1/C(=C\OC)C(=O)N(C)C] | SUSM-524 | 110 ± 16.4 | 9150 ± 1100 | NT | NT | NT | |

| | | | | | | |
|---|---|---|---|---|---|---|
| Mitragynine | 709 ± | 1530 ± | 6800 ± | 4090 ± | 4040 ± | 3.97 ± |
| | 91.2 | 143 | 721 | 273 | 417 | 9.46 |
| 7-Hydroxy Mitragynine | 71.8 ± | 450 ± | 243 ± | >10,000 | >10,000 | 41.8 ± |
| | 10.8 | 140 | 70.4 | | | 2.67 |
| Mitragynine pseudoindoxyl | 1.30 ± | 150 ± | 2.93 ± | NT | NT | 42.4 ± |
| | 0.125 | 20.5 | 0.443 | | | 6.59 |
| DAMGO | 3.38 ± | 2930 ± | 655 ± | NT | NT | 101 ± |
| | 0.436 | 879 | 107 | | | 3.91 |
| U69593 | 3260 ± | 2.30 ± | 6700 ± | NT | NT | |
| | 1100 | 0.335 | 739 | | | |
| SNC80 | 2570 ± | >10,000 | 5.56 ± | NT | NT | |
| | 395 | | 1.97 | | | |

NT = Not tested

TABLE A4
Summary of radioligand binding of mitragynine amides-data summary #2.

| | | | | | | MOR |
|---|---|---|---|---|---|---|
| | | MOR | KOR | DOR | Alpha2A | Emax ± |
| | | Kᵢ ± SEM | Kᵢ ± SEM | Kᵢ ± SEM | Kᵢ ± SEM | SEM (% |
| Structure | Code | (nM) | (nM) | (nM) | (nM) | DAMGO) |
| 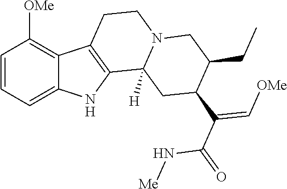 [SMILES: CC[C@@H]1CN2CCc3c([nH]c4cccc(OC)c34)[C@@H]2C[C@@H]1/C(=C\OC)C(=O)NC] | SUSM-25 | 3517 ± 763 | >10,000 | >10,000 | 6737 ± 1910 | — |
| 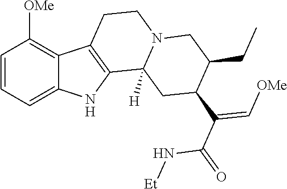 [SMILES: CCNC(=O)/C(=C/OC)[C@H]1C[C@H]2c3[nH]c4cccc(OC)c4c3CCN2C[C@H]1CC] | SUSM-32 | 4220 ± 752 | >10,000 | >10,000 | 3875 ± 984 | — |
| 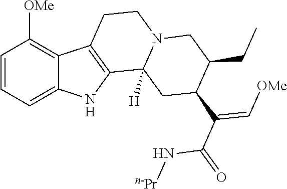 | SUSM-27 | 4560 ± 692 | >10,000 | >10,000 | 6452 ± 2591 | — |
| 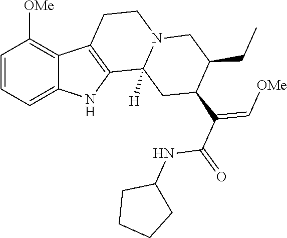 [SMILES: CC[C@@H]1CN2CCc3c([nH]c4cccc(OC)c34)[C@@H]2C[C@@H]1/C(=C\OC)C(=O)NC1CCCC1] | SUSM-506 | 1380 ± 176 | 5850 ± 657 | >10,000 | 5749 ± 568 | — |
| 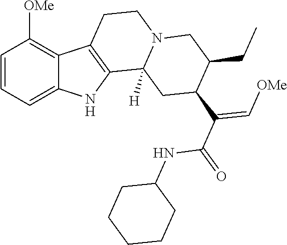 [SMILES: CC[C@@H]1CN2CCc3c([nH]c4cccc(OC)c34)[C@@H]2C[C@@H]1/C(=C\OC)C(=O)NC1CCCCC1] | SUSM-519 | 2183 ± 603 | 2790 ± 640 | >10,000 | 933 ± 242 | — |
| 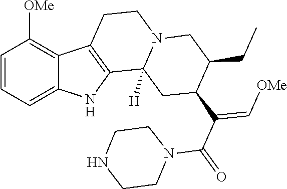 [SMILES: CC[C@@H]1CN2CCc3c([nH]c4cccc(OC)c34)[C@@H]2C[C@@H]1/C(=C\OC)C(=O)N1CCNCC1] | SUSM-562 | 4208 ± 558 | NT | >10,000 | NT | — |
| 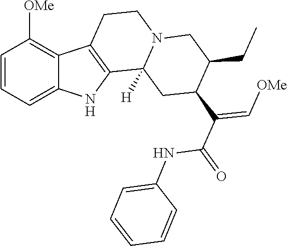 [SMILES: CC[C@@H]1CN2CCc3c([nH]c4cccc(OC)c34)[C@@H]2C[C@@H]1/C(=C\OC)C(=O)Nc1ccccc1] | SUSM-528 | 498 ± 118 | 6509 ± 4661 | >10,000 | 6584 ± 2316 | — |
| 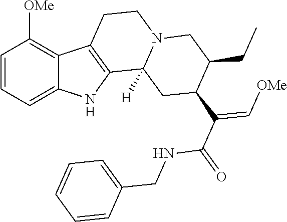 [SMILES: CC[C@@H]1CN2CCc3c([nH]c4cccc(OC)c34)[C@@H]2C[C@@H]1/C(=C\OC)C(=O)NCc1ccccc1] | SUSM-560 | 5320 ± 800 | NT | >10,000 | NT | — |
| 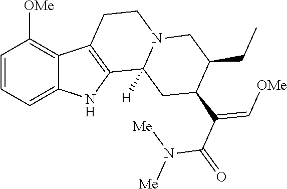 [SMILES: CC[C@@H]1CN2CCc3c([nH]c4cccc(OC)c34)[C@@H]2C[C@@H]1/C(=C\OC)C(=O)N(C)C] | SUSM-501 | 429 ± 75.7 | 3070 ± 569 | 6490 ± 3030 | 1810 ± 319 | −4.05 ± 4.03 |
| 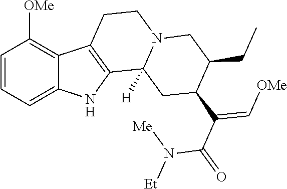 [SMILES: CC[C@@H]1CN2CCc3c([nH]c4cccc(OC)c34)[C@@H]2C[C@@H]1/C(=C\OC)C(=O)N(C)CC] | SUSM-520 | 452 ± 33 | 4780 ± 1140 | >10,000 | 938 ± 223 | — |
| 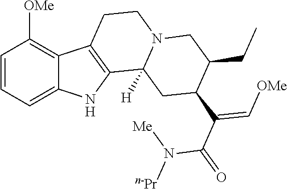 | SUSM-521 | 222 ± 33 | 2610 ± 588 | 4030 ± 797 | 1513 ± 676 | — |
| 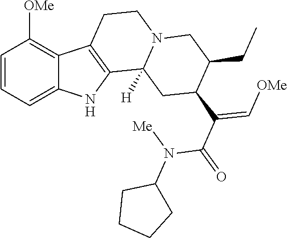 [SMILES: CC[C@@H]1CN2CCc3c([nH]c4cccc(OC)c34)[C@@H]2C[C@@H]1/C(=C\OC)C(=O)N(C)C1CCCC1] | SUSM-522 | 68 ± 7 | 4600 ± 132 | 6930 ± 1343 | 510 ± 82 | — |
| 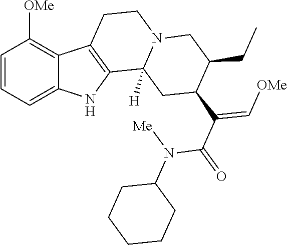 [SMILES: CC[C@@H]1CN2CCc3c([nH]c4cccc(OC)c34)[C@@H]2C[C@@H]1/C(=C\OC)C(=O)N(C)C1CCCCC1] | SUSM-523 | 49 ± 14 | 1947 ± 611 | 8285 ± 1024 | 187 ± 29 | — |
| 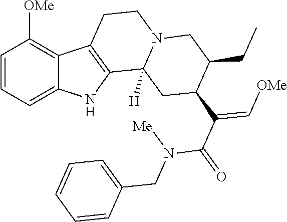 [SMILES: CC[C@@H]1CN2CCc3c([nH]c4cccc(OC)c34)[C@@H]2C[C@@H]1/C(=C\OC)C(=O)N(C)Cc1ccccc1] | SUSM-563 | 5749 ± 770 | NT | 4521 ± 1994 | NT | — |
| 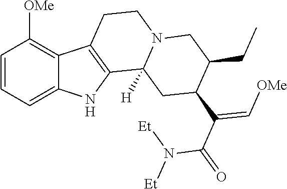 [SMILES: CC[C@@H]1CN2CCc3c([nH]c4cccc(OC)c34)[C@@H]2C[C@@H]1/C(=C\OC)C(=O)N(CC)CC] | SUSM-509 | 1130 ± 73.8 | >10,000 | >10,000 | 2731 ± 488 | — |
| 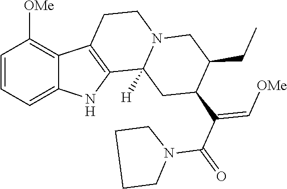 [SMILES: CC[C@@H]1CN2CCc3c([nH]c4cccc(OC)c34)[C@@H]2C[C@@H]1/C(=C\OC)C(=O)N1CCCC1] | SUSM-507 | 655 ± 76.6 | >10,000 | 6258 ± 3168 | 4993 ± 1016 | — |
| 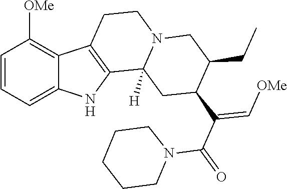 [SMILES: CC[C@@H]1CN2CCc3c([nH]c4cccc(OC)c34)[C@@H]2C[C@@H]1/C(=C\OC)C(=O)N1CCCCC1] | SUSM-518 | 244 ± 55 | 7460 ± 1420 | >10,000 | 523 ± 155 | — |
| 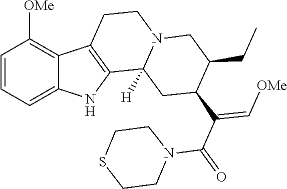 [SMILES: CC[C@@H]1CN2CCc3c([nH]c4cccc(OC)c34)[C@@H]2C[C@@H]1/C(=C\OC)C(=O)N1CCSCC1] | SUSM-564 | 134 ± 32 | NT | >10,000 | NT | — |
| 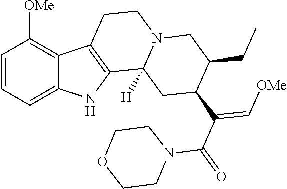 [SMILES: CC[C@@H]1CN2CCc3c([nH]c4cccc(OC)c34)[C@@H]2C[C@@H]1/C(=C\OC)C(=O)N1CCOCC1] | SUSM-527 | 1665 ± 427 | NT | >10,000 | NT | — |
| 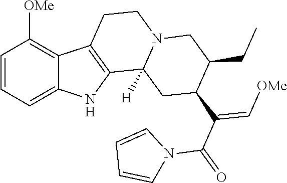 [SMILES: CC[C@@H]1CN2CCc3c([nH]c4cccc(OC)c34)[C@@H]2C[C@@H]1/C(=C\OC)C(=O)n1cccc1] | SUSM-526 | 719 ± 414 | >10,000 | >10,000 | >10,000 | — |
| 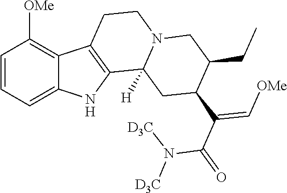 [SMILES: [2H]C([2H])([2H])N(C(=O)/C(=C/OC)[C@H]1C[C@H]2c3[nH]c4cccc(OC)c4c3CCN2C[C@H]1CC)C([2H])([2H])[2H]] | SUSM-566 | 330 ± 28 | 1804 ± 251 | 8303 ± 3300 | 1962 ± 535 | — |
| 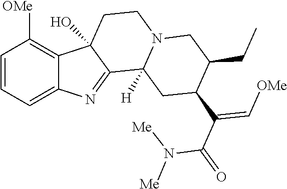 [SMILES: CC[C@@H]1CN2CC[C@@]3(O)C(=Nc4cccc(OC)c43)[C@@H]2C[C@@H]1/C(=C\OC)C(=O)N(C)C] | SUSM-524 | 90 ± 14 | 9149 ± 1103 | 802 ± 212 | >10,000 | — |

| | | | | | |
|---|---|---|---|---|---|
| Mitragynine | 353 ± | 264 ± | 1950 ± | 1353 ± | 3.97 ± |
| | 65 | 48 | 841 | 247 | 9.46 |
| 7-Hydroxy Mitragynine | 71.8 ± | 450 ± | 243 ± | >10,000 | 41.8 ± |
| | 10.8 | 140 | 70.4 | | 2.67 |
| DAMGO | 3.38 ± | 2930 ± | 655 ± | NT | 42.4 ± |
| | 0.436 | 879 | 107 | | 6.59 |
| U69593 | 3260 ± | 2.30 ± | 6700 ± | NT | |
| | 1100 | 0.335 | 739 | | |
| SNC80 | 2570 ± | >10,000 | 5.56 ± | NT | |
| | 395 | | 1.97 | | |

NT = Not tested

### RLM Stability Data:

Rat liver microsomal (RLM) stability for all the synthesized amides were done, data shows mono-alkylated amides (where N—H is intact) have higher stability, remains in the range of 30 minutes to >60 minutes. However, for di-alkylated amides stability decreases to <5 minute. Which suggest that di-alkylated amides are metabolized to mono-alkylated amides in RLM.

TABLE A2
RLM Stability Data

| Compound | RLM stability | Compound | RLM stability |
|---|---|---|---|
| Code | (min) | Code | (min) |
| SUSM-25 | >60 | SUSM-507 | <5 |
| SUSM-32 | >60 | SUSM-518 | <5 |
| SUSM-27 | >60 | SUSM-520 | <5 |
| SUSM-506 | 30 | SUSM-521 | <5 |
| SUSM-519 | 30 | SUSM-522 | <5 |
| SUSM-501 | 6 | SUSM-523 | <5 |
| SUSM-509 | <5 | SUSM-524 | <5 |

### Whole Liver Clearance

TABLE A3
Whole Liver Clearance

| | Whole liver clearance, |
|---|---|
| Code | CL int, H (mL/min*kg) |
| SUSM-25 | 8.9 |
| SUSM-32 | 11.4 |
| SUSM-27 | 20.8 |
| SUSM-506 | 36.4 |
| SUSM-519 | 38.4 |
| SUSM-501 | 128.8 |
| SUSM-509 | — |
| SUSM-507 | 833.8 |
| SUSM-518 | — |
| SUSM-520 | 879.7 |
| SUSM-521 | — |
| SUSM-522 | — |
| SUSM-523 | — |
| SUSM-526 | — |
| SUSM-528 | — |
| Mitragynine | 35.5 |
| DAMGO | — |
| U69593 | — |

### Experimental Procedures

Structural elucidation of the synthesized compounds was done through ¹H NMR, ¹³C NMR, and HRMS using Bruker model AMX 500 and Avance NEO 600 NMR spectrometers operating at 500 and 600 MHz in ¹H and 126 and 151 MHz in ¹³C, respectively. The chemical shift values were reported from trimethylsilane (TMS) in parts per million (ppm) using known solvent chemical shifts. The high-resolution mass spectra (HRMS) data and HPLC purity (>95%) were determined using an Agilent 1290 Infinity series ultraperformance liquid chromatography (UPLC) system equipped with photodiode array detector and quadrupole-time-of-flight (Q-TOF) Agilent 6540 mass spectrometer (Agilent Co, Santa Clara, CA, USA). The data was acquired by MassHunter data acquisition software (version B.09.00). The samples at a 10 M concentration were injected onto a Waters Acquity UPLC BEH C18 column (1.7 m, 2.1×100 mm). The gradient elution was performed using a mobile phase of 10 mM ammonium acetate (Sigma Aldrich Co., St. Louis, MO) buffer pH 3.5 (solvent A) and acetonitrile (solvent B, Sigma Aldrich Co.) at a flow rate of 0.5 mL/min. The gradient was started at 10% B and maintained till 1 min, then linearly increased to 90% B till 3 min, maintained at 90% B till 3.5 min, then decreased to 10% B from 3.5 to 4 min, and maintained at 10% B till 5 min. The column oven and autosampler temperatures were maintained at 45 and 4° C., respectively. The HPLC purity of samples were determined using the UV chromatogram extracted at a 254 nm wavelength. The high-resolution mass spectrometry chromatograms for each compound were extracted as base peak chromatograms at respective molecular (M+H)⁺ ion peaks. Column chromatography was carried out on silica gel (70-230 mesh, Merck) and Sephadex LH-20 (Mitsubishi Kagaku, Tokyo, Japan). TLC (silica gel 60 F₂₅₄, Sorbtech, Norcross, GA, USA) was used to monitor obtained fractions from column chromatography. Visualization of the TLC plates was achieved with a UV lamp (λ=254 and 365 nm) and anisaldehyde/acid spray reagent (MeOH-acetic acid-anisaldehyde-sulfuric acid, 85:9:1:5). All solvents and reagents used for plant extraction and compound isolation were of analytical grade (Fisher Scientific, Fair Lawn, NJ, USA).

### Experimental Procedure for the Synthesis of Mitragynine Acid

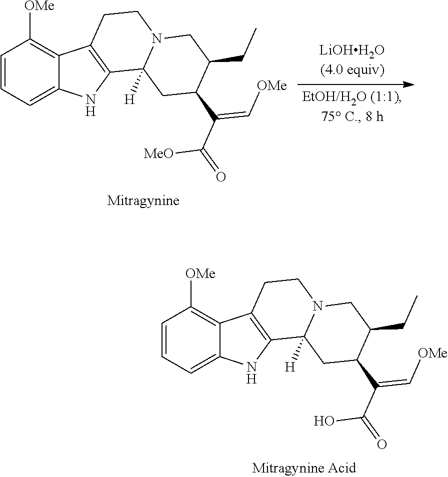 [SMILES: CC[C@@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@@H]2C[C@@H]1/C(=C\OC)C(=O)O]

To a stirred solution of Mitragynine (500 mg, 1.25 mmol) in EtOH/H₂O (1:1) (10 mL) was added LiOH·H₂O (119 mg, 5.0 mmol) at room temperature. The mixture was then heated at 75° C. for 8 h. After completion of the reaction (as monitored by TLC) the solvent was removed under vacuum. The crude residue was redissolved in DCM and purified using flash column chromatography (gradient: 0-10% MeOH in DCM) to yield: 381 mg (79%) of Mitragynine acid as a brown solid.

### Experimental Procedure for the Synthesis of Mitragynine Amide

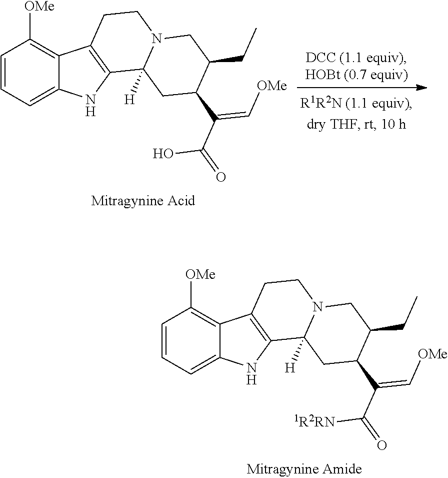

To a stirred solution of Mitragynine acid (100 mg, 0.26 mmol) in dry THF (3 mL) was added DCC (59 mg, 0.29 mmol), HOBt (24 mg, 0.18 mmol) and amine (0.29 mmol). The reaction mixture was then stirred at room temperature for 10 h. After completion (monitored by TLC), the resulting mixture was filtered through filter paper to remove DCC urea as side product. The filtrate was removed under reduced pressure. The crude residue was purified using flash column chromatography (gradient: 20-70% EtOAc in Hexanes) to yield mitragynine amide as desired product.

### Reaction Schemes

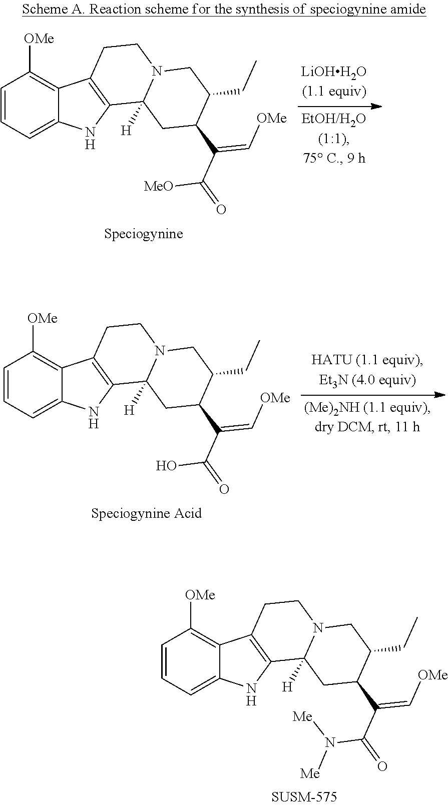 [SMILES: CC[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)O ; CC[C@H]1CN2CCc3c([nH]c4cccc(OC)c34)[C@@H]2C[C@@H]1/C(=C\OC)C(=O)N(C)C]

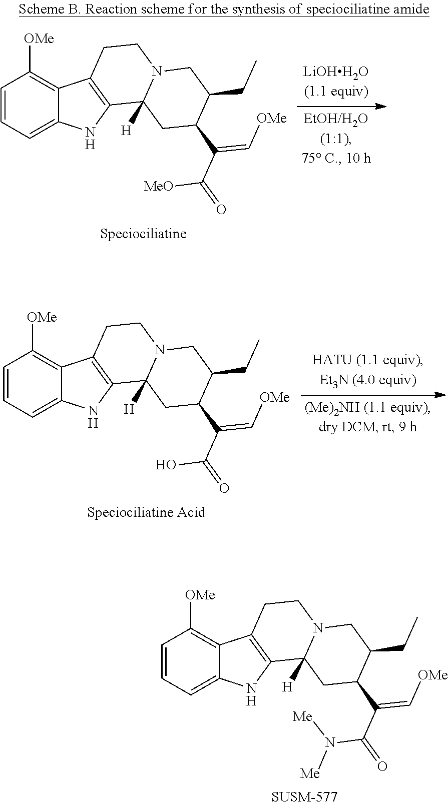 [SMILES: CC[C@@H]1CN2CCc3c([nH]c4cccc(OC)c34)[C@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)O ; CC[C@@H]1CN2CCc3c([nH]c4cccc(OC)c34)[C@H]2C[C@@H]1/C(=C\OC)C(=O)N(C)C]

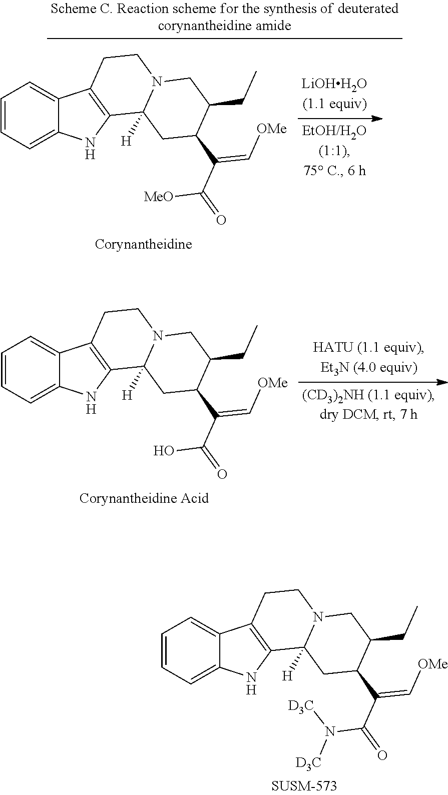 [SMILES: CC[C@@H]1CN2CCc3c([nH]c4ccccc34)[C@@H]2C[C@@H]1/C(=C\OC)C(=O)OC ; CC[C@@H]1CN2CCc3c([nH]c4ccccc34)[C@@H]2C[C@@H]1/C(=C\OC)C(=O)O ; [2H]C([2H])([2H])N(C(=O)/C(=C/OC)[C@H]1C[C@H]2c3[nH]c4ccccc4c3CCN2C[C@H]1CC)C([2H])([2H])[2H]]

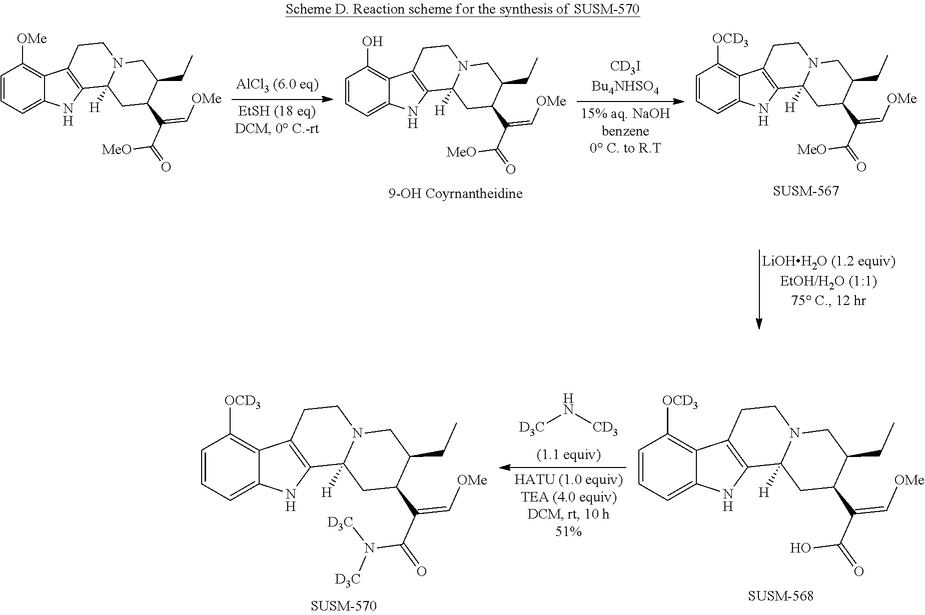

### Experimental Procedure for the Synthesis of Speciogynine Acid

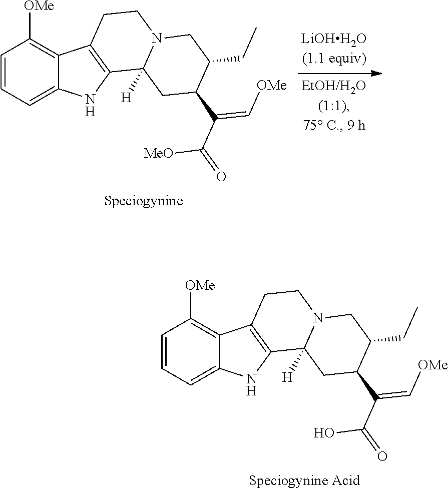 [SMILES: CC[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)O]

To a stirred solution of speciogynine (500 mg, 1.25 mmol) in EtOH/H₂O (1:1) (10 mL) was added LiOH·H₂O (57 mg, 1.37 mmol) at room temperature. The mixture was then heated at 75° C. for 9 h. After completion of the reaction (as monitored by TLC) the solvent was removed under vacuum. The crude residue was redissolved in DCM and purified using flash column chromatography (gradient: 0-10% MeOH in DCM) to yield: 361 mg (75%) of speciogynine acid as a yellow solid.

### Experimental Procedure for the Synthesis of Speciogynine Amide

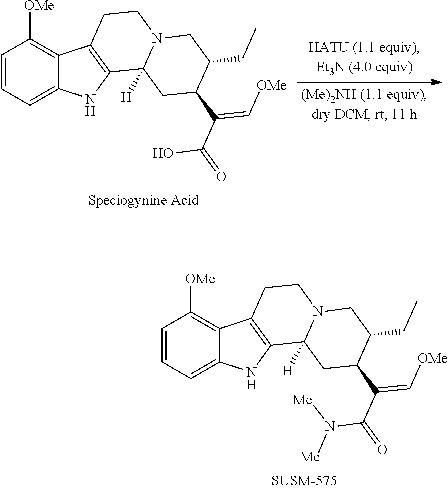 [SMILES: CC[C@H]1CN2CCc3c([nH]c4cccc(OC)c34)[C@@H]2C[C@@H]1/C(=C\OC)C(=O)O ; CC[C@H]1CN2CCc3c([nH]c4cccc(OC)c34)[C@@H]2C[C@@H]1/C(=C\OC)C(=O)N(C)C]

To a stirred solution of speciogynine acid (150 mg, 0.39 mmol) in dry DCM (3 mL) was added HATU (163 mg, 0.43 mmol), Et₃N (0.22 mL, 1.56 mmol) and di-methyl amine (0.43 mmol). The reaction mixture was then stirred at room temperature for 11 h. After completion (monitored by TLC) DCM (30 mL) was added to the reaction mixture followed by addition of H₂O (10 mL). The organic layer was removed under reduced pressure. The crude residue was purified using flash column chromatography (gradient: 0-50% EtOAc in hexanes) to yield speciogynine amide (SUSM-575) as desired product in 41% yield.

### Experimental Procedure for the Synthesis of Speciociliatine Acid

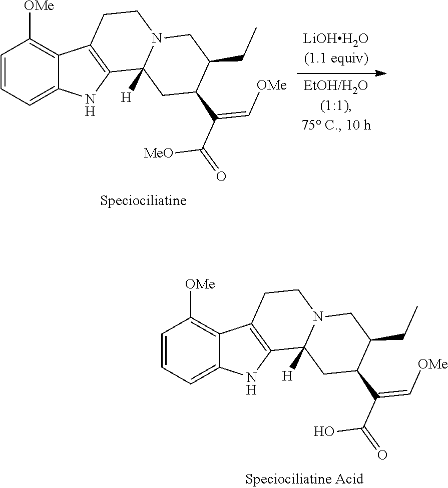 [SMILES: CC[C@@H]1CN2CCc3c([nH]c4cccc(OC)c34)[C@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)O]

To a stirred solution of speciociliatine (500 mg, 1.25 mmol) in EtOH/H₂O (1:1) (10 mL) was added LiOH·H₂O (57 mg, 1.37 mmol) at room temperature. The mixture was then heated at 75° C. for 10 h. After completion of the reaction (as monitored by TLC) the solvent was removed under vacuum. The crude residue was redissolved in DCM and purified using flash column chromatography (gradient: 0-10% MeOH in DCM) to yield: 385 mg (80%) of speciociliatine acid as a pale yellow solid.

### Experimental Procedure for the Synthesis of Speciociliatine Amide

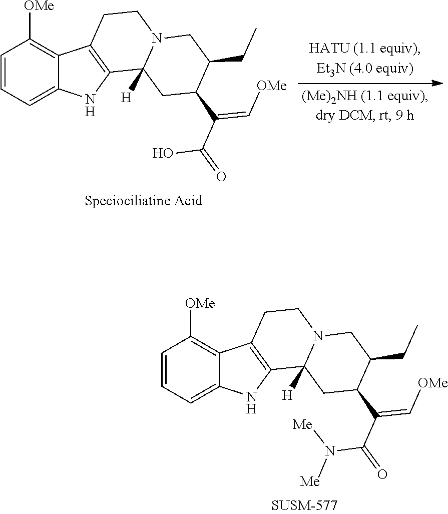 [SMILES: CC[C@@H]1CN2CCc3c([nH]c4cccc(OC)c34)[C@H]2C[C@@H]1/C(=C\OC)C(=O)O ; CC[C@@H]1CN2CCc3c([nH]c4cccc(OC)c34)[C@H]2C[C@@H]1/C(=C\OC)C(=O)N(C)C]

To a stirred solution of speciociliatine acid (150 mg, 0.39 mmol) in dry DCM (3 mL) was added HATU (163 mg, 0.43 mmol), Et₃N (0.22 mL, 1.56 mmol) and di-methyl amine (0.43 mmol). The reaction mixture was then stirred at room temperature for 9 h. After completion (monitored by TLC) DCM (30 mL) was added to the reaction mixture followed by addition of H₂O (10 mL). The organic layer was removed under reduced pressure. The crude residue was purified using flash column chromatography (gradient: 0-55% EtOAc in hexanes) to yield speciociliatine amide (SUSM-577) as desired product in 43% yield.

### Experimental Procedure for the Synthesis of 7-OH-Mitragynine Amide

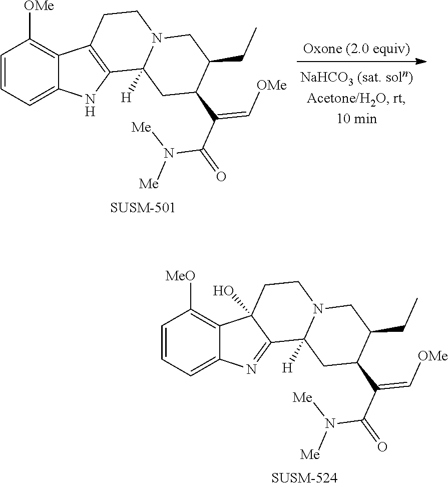 [SMILES: CC[C@@H]1CN2CCc3c([nH]c4cccc(OC)c34)[C@@H]2C[C@@H]1/C(=C\OC)C(=O)N(C)C ; CC[C@@H]1CN2CC[C@@]3(O)C(=Nc4cccc(OC)c43)[C@@H]2C[C@@H]1/C(=C\OC)C(=O)N(C)C]

SUSM-501 (100 mg, 0.24 mmol) was dissolved in acetonitrile (5 mL), then water (1 mL) was added. The resulting suspension was cooled to 0° C., and the following solution was added slowly over the course of several minutes: oxone (73 mg, 0.28 mmol) in 2 mL acetonitrile. The reaction mixture was stirred at room temperature for 10 min, then saturated aqueous NaHCO₃ solution was added, and the mixture extracted with EtOAc. The organic phase was rinsed with brine (6 mL) and dried over anhydrous Na₂SO₄, and then it was evaporated under reduced pressure. The residue was dissolved in DCM and purified using flash column chromatography (gradient: 0-75% EtOAc in hexanes). The fractions containing the product were evaporated to yield 35 mg (32%) of 7-OH-Mitragynine amide (SUSM-524) as a white solid product.

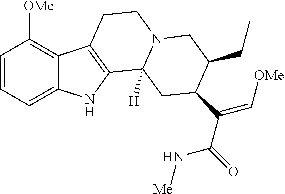 [SMILES: CC[C@@H]1CN2CCc3c([nH]c4cccc(OC)c34)[C@@H]2C[C@@H]1/C(=C\OC)C(=O)NC]

(E)-2-((2S,3S,12bS)-3-ethyl-8-methoxy-1,2,3,4,6,7,12,12b-octahydroindolo[2,3-a]quinolizin-2-yl)-3-methoxy-N-methylacrylamide (SUSM-25). Yield: 31% (32 mg from 100 mg); a white solid; R_{f}=0.43 (MeOH:EtOAc, 0.5:9.5, v/v); ¹H NMR (600 MHz, CDCl₃): δ (ppm) 7.93 (s, 1H), 6.99 (t, J=7.9 Hz, 1H), 6.96 (s, 1H), 6.91 (d, J=7.4 Hz, 1H), 6.45 (d, J=7.7 Hz, 1H), 5.73 (s, 1H), 3.87 (s, 3H), 3.64 (s, 3H), 3.16-3.09 (m, 2H), 3.02-2.95 (m, 2H), 2.92-2.87 (m, 2H), 2.84 (d, J=4.7 Hz, 3H), 2.54-2.50 (m, 1H), 2.43-2.33 (m, 2H), 1.91 (d, J=13.0 Hz, 1H), 1.79-1.77 (m, 1H), 1.62-1.61 (m, 1H), 1.27-1.25 (m, 1H), 0.88 (t, J=7.4 Hz, 3H); ¹³C NMR (151 MHz, CDCl₃): δ (ppm)=170.2, 154.6, 154.4, 137.5, 133.7, 121.9, 117.7, 116.1, 107.9, 104.5, 99.8, 77.4, 77.2, 76.9, 61.2, 61.1, 57.8, 55.5, 53.8, 40.8, 40.6, 30.2, 26.8, 24.0, 19.5, 13.0. MS (ESI⁺): m/z=398.2 [M+H]⁺.

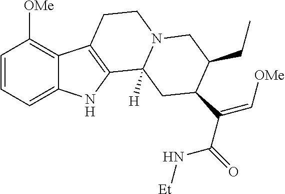 [SMILES: CCNC(=O)/C(=C/OC)[C@H]1C[C@H]2c3[nH]c4cccc(OC)c4c3CCN2C[C@H]1CC]

(E)-N-ethyl-2-((2S,3S,12bS)-3-ethyl-8-methoxy-1,2,3,4,6,7,12,12b-octahydroindolo[2,3-a]quinolizin-2-yl)-3-methoxyacrylamide (SUSM-32). Yield: 37% (39 mg from 100 mg); a white solid, R_{f}=0.41 (MeOH:EtOAc, 0.5:9.5, v/v); ¹H NMR (600 MHz, CDCl₃): δ (ppm) 7.95 (s, 1H), 6.99 (t, J=8.0 Hz, 1H), 6.91 (d, J=8.5 Hz, 2H), 6.44 (d, J=7.7 Hz, 1H), 5.67 (t, J=5.5 Hz, 1H), 3.87 (s, 3H), 3.65 (s, 3H), 3.34-3.30 (m, 2H), 3.18-3.08 (m, 2H), 3.02-2.97 (m, 2H), 2.92-2.86 (m, 3H), 2.52 (td, J=11.4, 4.1 Hz, 1H), 2.42 (dd, J=11.6, 3.1 Hz, 1H), 2.35 (q, J=12.4 Hz, 1H), 1.93 (dt, J=13.1, 3.1 Hz, 1H), 1.89-1.82 (m, 1H), 1.80-1.74 (m, 1H), 1.61 (dt, J=11.2, 3.2 Hz, 1H), 1.16 (t, J=7.3 Hz, 3H), 0.88 (t, J=7.4 Hz, 3H); ¹³C NMR (151 MHz, CDCl₃): δ (ppm)=169.3, 162. 6, 154.5, 153.9, 137.3, 133.6, 121.8, 117.6, 116.3, 107.7, 104.3, 77.3, 77.0, 76.8, 61.1, 60.9, 57.7, 55.3, 53.7, 40.7, 36.5, 34.7, 31.4, 30.1, 23.9, 19.3, 12.9. MS (ESI⁺): m/z=412.3 [M+H]⁺.

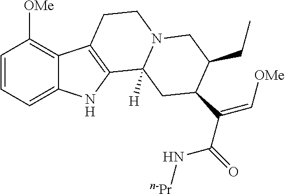

(E)-2-((2S,3S,12bS)-3-ethyl-8-methoxy-1,2,3,4,6,7,12,12b-octahydroindolo[2,3-a]quinolizin-2-yl)-3-methoxy-N-propylacrylamide (SUSM-27). Yield: 33% (36 mg from 100 mg); a yellow solid; R_{f}=0.44 (MeOH:EtOAc, 0.5:9.5, v/v); ¹H NMR (600 MHz, CDCl₃): δ (ppm) 7.83 (s, 1H), 6.99 (t, J=7.9 Hz, 1H), 6.91 (d, J=9.2 Hz, 2H), 6.45 (d, J=7.7 Hz, 1H), 5.66 (t, J=5.8 Hz, 1H), 4.09 (d, J=7.3 Hz, 1H), 3.87 (s, 3H), 3.65 (s, 3H), 3.49-3.47 (m, 1H), 3.26 (q, J=6.5 Hz, 2H), 3.17-3.08 (m, 1H), 3.02-2.95 (m, 2H), 2.92-2.88 (m, 2H), 2.55-2.50 (m, 1H), 2.43-2.41 (m, 1H), 2.38-2.32 (m, 1H), 1.96-1.92 (m, 2H), 1.81-1.76 (m, 1H), 1.71-1.69 (m, 1H), 1.63-1.59 (m, 1H), 0.93 (t, J=7.4 Hz, 3H), 0.88 (t, J=7.4 Hz, 3H); ¹³C NMR (151 MHz, CDCl₃): δ (ppm)=168.7, 153.5, 148.9, 137.3, 133.7, 121.7, 117.6, 115.8, 107.7, 104.3, 99.7, 77.2, 77.0, 76.8, 60.3, 55.3, 53.7, 40.4, 34.7, 31.6, 31.1, 24.3, 22.7, 21.7, 19.3, 14.2, 12.2. MS (ESI⁺): m/z=426.3 [M+H]⁺.

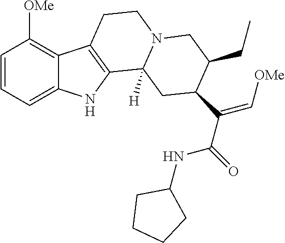 [SMILES: CC[C@@H]1CN2CCc3c([nH]c4cccc(OC)c34)[C@@H]2C[C@@H]1/C(=C\OC)C(=O)NC1CCCC1]

(E)-N-cyclopentyl-2-((2S,3S,12bS)-3-ethyl-8-methoxy-1,2,3,4,6,7,12,12b-octahydroindolo[2,3-a]quinolizin-2-yl)-3-methoxyacrylamide (SUSM-506). Yield: 30% (35 mg from 100 mg); a white solid; R_{f}=0.41 (MeOH:EtOAc, 0.5:9.5, v/v); ¹H NMR (600 MHz, CDCl₃): δ (ppm) 7.90 (s, 1H), 6.99 (t, J=7.9 Hz, 1H), 6.91 (d, J=7.9 Hz, 1H), 6.87 (s, 1H), 6.45 (d, J=7.7 Hz, 1H), 5.54 (s, 1H), 4.25-4.20 (m, 1H), 3.87 (s, 3H), 3.65 (s, 3H), 3.17-3.10 (m, 2H), 3.02-2.95 (m, 2H), 2.92-2.87 (m, 2H), 2.52 (td, J=11.5, 4.3 Hz, 1H), 2.43-2.41 (m, 1H), 2.33 (td, J=12.9, 11.3 Hz, 1H), 2.04-1.99 (m, 2H), 1.94 (dt, J=13.1, 3.1 Hz, 1H), 1.79-1.73 (m, 1H), 1.69-1.66 (m, 1H), 1.62-1.60 (m, 2H), 1.41-1.36 (m, 2H), 1.27-1.24 (m, 1H), 0.88 (t, J=7.3 Hz, 3H); ¹³C NMR (151 MHz, CDCl₃): δ (ppm)=169.1, 154.6, 153.7, 137.5, 133.7, 121.9, 117.7, 116.7, 107.9, 104.5, 99.8, 77.4, 77.2, 76.9, 61.3, 61.0, 57.9, 55.5, 53.9, 51.6, 40.9, 34.8, 33.3, 31.7, 30.2, 25.4, 24.1, 23.9, 22.8, 19.5, 13.1. MS (ESI⁺): m/z=452.3 [M+H]⁺.

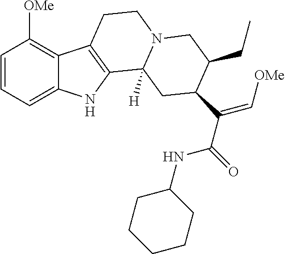 [SMILES: CC[C@@H]1CN2CCc3c([nH]c4cccc(OC)c34)[C@@H]2C[C@@H]1/C(=C\OC)C(=O)NC1CCCCC1]

(E)-N-cyclohexyl-2-((2S,3S,12bS)-3-ethyl-8-methoxy-1,2,3,4,6,7,12,12b-octahydroindolo[2,3-a]quinolizin-2-yl)-3-methoxyacrylamide (SUSM-519). Yield: 27% (32 mg from 100 mg); a brown solid; R_{f}=0.46 (MeOH:EtOAc, 0.5:9.5, v/v); ¹H NMR (600 MHz, CDCl₃): δ (ppm) 7.91 (s, 1H), 6.99 (t, J=7.9 Hz, 1H), 6.91 (d, J=8.1 Hz, 1H), 6.85 (s, 1H), 6.44 (d, J=7.8 Hz, 1H), 5.48 (d, J=8.1 Hz, 1H), 3.87 (s, 3H), 3.81-3.80 (m, 1H), 3.64 (s, 3H), 3.17-3.07 (m, 2H), 3.03-2.95 (m, 2H), 2.90 (dq, J=13.5, 4.7, 3.7 Hz, 2H), 2.52 (td, J=11.5, 4.3 Hz, 1H), 2.41 (dd, J=11.3, 3.1 Hz, 1H), 2.32 (td, J=12.9, 11.3 Hz, 1H), 1.94 (td, J=10.6, 5.6 Hz, 3H), 1.81-1.74 (m, 1H), 1.73-1.66 (m, 2H), 1.40-1.35 (m, 3H), 1.27-1.23 (m, 1H), 1.21-1.09 (m, 4H), 0.88 (t, J=7.4 Hz, 3H); ¹³C NMR (151 MHz, CDCl₃): δ (ppm)=168.5, 154.5, 153.3, 137.3, 133.6, 121.8, 117.6, 116.7, 107.7, 104.3, 99.7, 77.3, 77.0, 76.8, 61.2, 60.9, 57.8, 55.3, 53.8, 48.3, 40.8, 40.7, 34.0, 33.3, 33.2, 30.9, 25.6, 24.9, 23.9, 19.4, 13.0. MS (ESI⁺): m/z=466.3 [M+H]⁺.

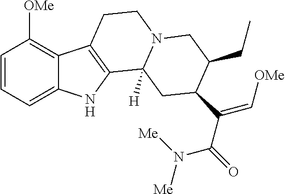 [SMILES: CC[C@@H]1CN2CCc3c([nH]c4cccc(OC)c34)[C@@H]2C[C@@H]1/C(=C\OC)C(=O)N(C)C]

(E)-2-((2S,3S,12bS)-3-ethyl-8-methoxy-1,2,3,4,6,7,12,12b-octahydroindolo[2,3-a]quinolizin-2-yl)-3-methoxy-N,N-dimethylacrylamide (SUSM-501). Yield: 40% (42 mg from 100 mg); a white solid; R_{f}=0.48 (MeOH:EtOAc, 0.5:9.5, v/v); ¹H NMR (600 MHz, CDCl₃): δ (ppm) 7.87 (s, 1H), 6.98 (t, J=7.9 Hz, 1H), 6.90 (d, J=8.3 Hz, 1H), 6.44 (d, J=7.8 Hz, 1H), 6.17 (s, 1H), 3.87 (s, 3H), 3.60 (s, 3H), 3.17-3.07 (m, 2H), 3.04 (s, 6H), 3.00-2.94 (m, 2H), 2.91-2.88 (m, 2H), 2.52 (td, J=11.4, 4.2 Hz, 1H), 2.38 (d, J=11.0 Hz, 1H), 2.18-2.12 (m, 1H), 2.01 (d, J=13.0 Hz, 1H), 1.88-1.84 (m, 1H), 1.32-1.25 (m, 2H), 0.91 (t, J=7.4 Hz, 3H); ¹³C NMR (151 MHz, CDCl₃): δ (ppm)=172.1, 154.6, 149.6, 137.4, 133.8, 121.8, 117.7, 115.5, 107.8, 104.5, 99.8, 77.4, 77.2, 76.9, 61.1, 60.5, 57.7, 55.4, 53.8, 41.8, 40.5, 34.8, 30.2, 24.1, 19.5, 14.3, 13.1. MS (ESI⁺): m/z=412.3 [M+H]⁺.

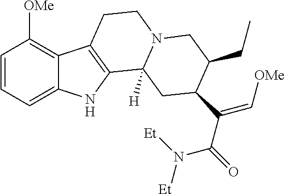 [SMILES: CC[C@@H]1CN2CCc3c([nH]c4cccc(OC)c34)[C@@H]2C[C@@H]1/C(=C\OC)C(=O)N(CC)CC]

(E)-N-ethyl-2-((2S,3S,12bS)-3-ethyl-8-methoxy-1,2,3,4,6,7,12,12b-octahydroindolo[2,3-a]quinolizin-2-yl)-3-methoxy-N-methylacrylamide (SUSM-520). Yield: 34% (37 mg from 100 mg); a yellow solid; R_{f}=0.49 (MeOH:EtOAc, 0.5:9.5, v/v); ¹H Et NMR (600 MHz, CDCl₃): δ (ppm) 7.93 (s, 1H), 7.01 (t, J=7.9 Hz, 1H), 6.93 (d, J=8.1 Hz, 1H), 6.46 (d, J=7.7 Hz, 1H), 6.17 (s, 1H), 3.89 (s, 3H), 3.62 (s, 3H), 3.57-3.43 (m, 2H), 3.18 (d, J=10.1 Hz, 1H), 3.12 (ddd, J=11.6, 5.3, 2.7 Hz, 1H), 3.02 (s, 3H), 2.98 (dd, J=17.4, 3.1 Hz, 1H), 2.92 (ddd, J=12.6, 6.1, 2.7 Hz, 2H), 2.54 (td, J=11.4, 4.2 Hz, 1H), 2.41 (d, J=10.9 Hz, 1H), 2.17 (q, J=12.8 Hz, 1H), 2.04 (d, J=13.1 Hz, 1H), 1.97-1.83 (m, 1H), 1.41-1.25 (m, 2H), 1.19 (t, J=7.1 Hz, 3H), 0.93 (t, J=7.4 Hz, 3H); ¹³C NMR (151 MHz, CDCl₃): δ (ppm)=171.5, 154.5, 148.7, 137.3, 133.7, 121.7, 117.6, 115.8, 107.7, 104.3, 99.7, 77.2, 77.0, 76.8, 61.0, 60.3, 57.6, 55.3, 53.7, 41.7, 40.4, 31.6, 30.1, 23.9, 22.7, 19.3, 14.1, 12.9. MS (ESI⁺): m/z=426.3 [M+H]⁺.

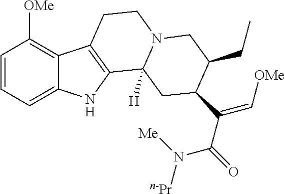

(E)-2-((2S,3S,12bS)-3-ethyl-8-methoxy-1,2,3,4,6,7,12,12b-octahydroindolo[2,3-a]quinolizin-2-yl)-3-methoxy-N-methyl-N-propylacrylamide (SUSM-521). Yield: 28% (32 mg from 100 mg); a white solid; R_{f}=0.47 (MeOH:EtOAc, 0.5:9.5, v/v); ¹H NMR (600 MHz, CDCl₃): δ (ppm) 7.93 (s, 1H), 6.98 (t, J=7.9 Hz, 1H), 6.90 (d, J=8.2 Hz, 1H), 6.44 (d, J=7.7 Hz, 1H), 6.14 (s, 1H), 3.86 (s, 3H), 3.59 (s, 3H), 3.45-3.39 (m, 1H), 3.37-3.32 (m, 1H), 3.17-3.08 (m, 2H), 3.03-2.96 (m, 4H), 2.91-2.85 (m, 2H), 2.52 (dt, J=11.4, 5.6 Hz, 1H), 2.37 (dd, J=11.3, 3.0 Hz, 1H), 2.14 (q, J=12.5 Hz, 1H), 2.04-1.98 (m, 1H), 1.96-1.81 (m, 2H), 1.63-1.56 (m, 2H), 1.37-1.20 (m, 2H), 0.90 (dd, J=7.4, 3.7 Hz, 6H); ¹³C NMR (151 MHz, CDCl₃): δ (ppm)=171.7, 154.5, 148.9, 137.3, 133.7, 121.7, 117.6, 115.8, 107.7, 104.3, 99.7, 77.2, 77.0, 76.8, 60.3, 55.3, 53.7, 40.4, 34.7, 31.6, 30.1, 25.3, 22.7, 20.7, 19.3, 14.1, 12.9, 11.3. MS (ESI⁺): m/z=440.3 [M+H]⁺.

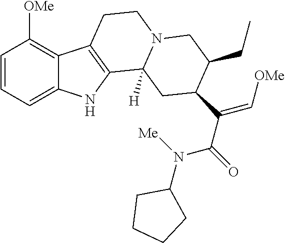 [SMILES: CC[C@@H]1CN2CCc3c([nH]c4cccc(OC)c34)[C@@H]2C[C@@H]1/C(=C\OC)C(=O)N(C)C1CCCC1]

(E)-2-((2S,3S,12bS)-3-ethyl-8-methoxy-1,2,3,4,6,7,12,12b-octahydroindolo[2,3-a]quinolizin-2-yl)-3-methoxy-N-methylacrylamide (SUSM-522). Yield: 30% (36 mg from 100 mg); a brown solid; R_{f}=0.51 (MeOH:EtOAc, 0.5:9.5, v/v); ¹H NMR (600 MHz, CDCl₃): δ ^{N} (ppm) 7.98 (s, 1H), 6.98 (t, J=7.9 Hz, 1H), 6.91 (d, J=8.0 Hz, 1H), 6.44 (d, J=7.7 Hz, 1H), 6.14 (s, 1H), 4.65 (t, J=8.4 Hz, 1H), 3.86 (s, 3H), 3.59 (s, 3H), 3.15-3.08 (m, 2H), 2.99-2.94 (m, 2H), 2.91-2.88 (m, 2H), 2.85 (s, 3H), 2.52 (td, J=11.5, 4.3 Hz, 1H), 2.39-2.37 (m, 1H), 2.17-2.11 (m, 1H), 2.00 (dt, J=12.9, 3.2 Hz, 1H), 1.88 (ddt, J=13.5, 11.3, 7.0 Hz, 2H), 1.74-1.70 (m, 2H), 1.59 (dt, J=6.0, 2.7 Hz, 3H), 1.31-1.28 (m, 1H), 0.90 (t, J=7.4 Hz, 3H); ¹³C NMR (151 MHz, CDCl₃): δ (ppm)=172.0, 154.4, 148.5, 137.3, 133.8, 121.7, 117.6, 116.3, 107.6, 104.4, 99.6, 77.3, 77.1, 76.8, 61.0, 60.3, 57.6, 55.3, 53.7, 41.8, 40.5, 34.7, 34.5, 31.6, 30.1, 29.1, 25.3, 24.6, 24.6, 24.0, 22.7, 20.7, 19.4, 14.1, 13.0, 11.4. MS (ESI⁺): m/z=466.3 [M+H]⁺.

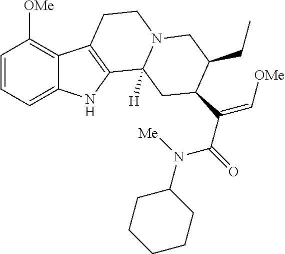 [SMILES: CC[C@@H]1CN2CCc3c([nH]c4cccc(OC)c34)[C@@H]2C[C@@H]1/C(=C\OC)C(=O)N(C)C1CCCCC1]

(E)-N-cyclohexyl-2-((2S,3S,12bS)-3-ethyl-8-methoxy-1,2,3,4,6,7,12,12b-octahydroindolo[2,3-a]quinolizin-2-yl)-3-methoxy-N-methylacrylamide (SUSM-523). Yield: 26% (32 mg from 100 mg); a yellow solid; R_{f}=0.53 (MeOH:EtOAc, 0.5:9.5, v/v); ¹H NMR (600 MHz, CDCl₃): δ (ppm) 7.95 (s, 1H), 6.98 (t, J=7.9 Hz, 1H), 6.91 (d, J=8.0 Hz, 1H), 6.44 (d, J=7.6 Hz, 1H), 6.13 (s, 1H), 4.14-4.12 (m, 1H), 3.86 (s, 3H), 3.16-3.08 (m, 2H), 2.99-2.94 (m, 2H), 2.91-2.87 (m, 5H), 2.54-2.49 (m, 1H), 2.39-2.36 (m, 1H), 2.17-2.13 (m, 1H), 2.03-2.01 (m, 1H), 1.89-1.82 (m, 4H), 1.69-1.61 (m, 4H), 1.53-1.47 (m, 2H), 1.35-1.30 (m, 3H), 0.91 (t, J=7.4 Hz, 3H); ¹³C NMR (151 MHz, CDCl₃): δ (ppm)=170.5, 152.5, 151.3, 136.3, 132.6, 121.8, 117.6, 116.7, 107.7, 104.3, 99.7, 77.3, 77.0, 76.8, 61.2, 60.9, 57.8, 55.3, 53.8, 48.3, 40.8, 40.7, 34.0, 33.3, 33.2, 30.9, 25.6, 24.9, 22.9, 19.6, 12.3, 13.1. MS (ESI⁺): m/z=480.3 [M+H]⁺.

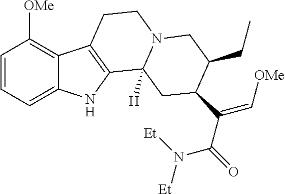 [SMILES: CC[C@@H]1CN2CCc3c([nH]c4cccc(OC)c34)[C@@H]2C[C@@H]1/C(=C\OC)C(=O)N(CC)CC]

(E)-N,N-diethyl-2-((2S,3S,12bS)-3-ethyl-8-methoxy-1,2,3,4,6,7,12,12b-octahydroindolo[2,3-a]quinolizin-2-yl)-3-methoxyacrylamide (SUSM-509). Yield: 31% (35 mg from 100 mg); a white solid; R_{f}=0.58 (MeOH:EtOAc, 0.5:9.5, v/v); ¹H NMR (600 MHz, CDCl₃): δ (ppm) 7.86 (s, 1H), 6.98 (t, J=7.9 Hz, 1H), 6.90 (d, J=7.6 Hz, 1H), 6.44 (d, J=7.7 Hz, 1H), 6.13 (s, 1H), 3.87 (s, 3H), 3.59 (s, 3H), 3.49-3.39 (m, 3H), 3.16 (d, J=11.1 Hz, 1H), 3.13-3.08 (m, 1H), 2.99-2.94 (m, 1H), 2.91-2.86 (m, 2H), 2.52 (td, J=11.4, 4.3 Hz, 1H), 2.40-2.36 (m, 1H), 2.17-2.10 (m, 1H), 2.02-1.99 (m, 1H), 1.88-1.83 (m, 1H), 1.33-1.25 (m, 2H), 1.17-1.15 (m, 6H), 0.91 (t, J=7.4 Hz, 3H); ¹³C NMR (151 MHz, CDCl₃): δ (ppm)=171.1, 154.5, 147.7, 137.3, 133.7, 121.7, 117.6, 116.2, 107.7, 104.3, 99.7, 61.0, 60.3, 55.3, 53.7, 41.7, 40.4, 34.7, 31.6, 30.2, 25.3, 24.0, 22.7, 19.3, 14.1, 12.9. MS (ESI⁺): m/z=440.3 [M+H]⁺.

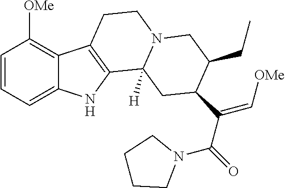 [SMILES: CC[C@@H]1CN2CCc3c([nH]c4cccc(OC)c34)[C@@H]2C[C@@H]1/C(=C\OC)C(=O)N1CCCC1]

(E)-2-((2S,3S,12bS)-3-ethyl-8-methoxy-1,2,3,4,6,7,12,12b-octahydroindolo[2,3-a]quinolizin-2-yl)-3-methoxy-1-(pyrrolidin-1-yl)prop-2-en-1-one (SUSM-507). Yield: 33% (37 mg from 100 mg); a white solid; R_{f}=0.42 (MeOH:EtOAc, 0.5:9.5, v/v); ¹H NMR (600 MHz, CDCl₃): δ (ppm) 7.94 (s, 1H), 6.98 (t, J=7.9 Hz, 1H), 6.91 (d, J=7.9 Hz, 1H), 6.44 (d, J=7.7 Hz, 1H), 6.28 (s, 1H), 3.86 (s, 3H), 3.60 (s, 3H), 3.53-3.50 (m, 4H), 3.16-3.09 (m, 1H), 2.98-2.94 (m, 3H), 2.91-2.88 (m, 1H), 2.52 (td, J=11.4, 4.2 Hz, 1H), 2.39 (d, J=10.5 Hz, 1H), 2.21-2.15 (m, 1H), 2.01-1.98 (m, 1H), 1.90-1.85 (m, 3H), 1.66-1.63 (m, 1H), 1.30-1.25 (m, 1H), 0.90 (t, J=7.4 Hz, 3H); ¹³C NMR (151 MHz, CDCl₃): δ (ppm)=170.3, 154.4, 150.1, 137.3, 133.8, 121.7, 117.6, 117.2, 107.6, 104.4, 99.6, 77.3, 77.0, 76.8, 61.0, 60.4, 57.6, 55.3, 53.7, 41.4, 40.6, 31.6, 30.1, 25.3, 24.0, 22.7, 19.4, 14.1, 13.0. MS (ESI⁺): m/z=438.3 [M+H]⁺.

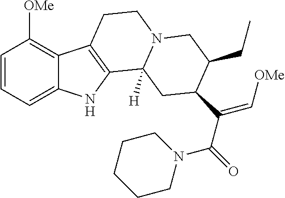 [SMILES: CC[C@@H]1CN2CCc3c([nH]c4cccc(OC)c34)[C@@H]2C[C@@H]1/C(=C\OC)C(=O)N1CCCCC1]

(E)-2-((2S,3S,12bS)-3-ethyl-8-methoxy-1,2,3,4,6,7,12,12b-octahydroindolo[2,3-OMe a]quinolizin-2-yl)-3-methoxy-1-(piperidin-1-yl)prop-2-en-1-one (SUSM-518). Yield: 28% (32 mg from 100 mg); a white solid; R_{f}=0.40 (MeOH:EtOAc, 0.5:9.5, v/v); ¹H NMR (600 MHz, CDCl₃): δ (ppm) 7.91 (s, 1H), 6.98 (t, J=7.9 Hz, 1H), 6.91 (d, J=8.0 Hz, 1H), 6.44 (d, J=7.7 Hz, 1H), 6.16 (s, 1H), 3.86 (s, 3H), 3.62-3.53 (m, 7H), 3.17-3.08 (m, 2H), 2.98-2.94 (m, 2H), 2.91-2.87 (m, 2H), 2.52 (td, J=11.4, 4.2 Hz, 1H), 2.38 (dd, J=8.6, 1.3 Hz, 1H), 2.18-2.12 (m, 1H), 2.04-2.01 (m, 1H), 1.89-1.84 (m, 1H), 1.67-1.65 (m, 1H), 1.58-1.53 (m, 5H), 0.90 (t, J=7.4 Hz, 3H); ¹³C NMR (151 MHz, CDCl₃): δ (ppm)=170.7, 154.6, 148.9, 137.4, 133.8, 121.8, 117.7, 115.7, 107.8, 104.5, 99.8, 77.4, 77.2, 76.9, 61.1, 60.5, 57.7, 55.4, 53.8, 47.6, 42.6, 40.5, 30.2, 26.3, 25.6, 24.9, 24.6, 24.1, 19.5, 13.0. MS (ESI⁺): m/z=452.3 [M+H]⁺.

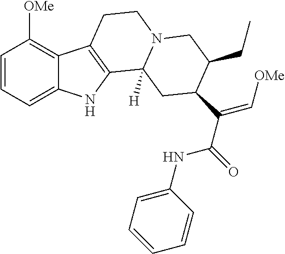 [SMILES: CC[C@@H]1CN2CCc3c([nH]c4cccc(OC)c34)[C@@H]2C[C@@H]1/C(=C\OC)C(=O)Nc1ccccc1]

(E)-2-((2S,3S,12bS)-3-ethyl-8-methoxy-1,2,3,4,6,7,12,12b-octahydroindolo[2,3-a]quinolizin-2-yl)-3-methoxy-N-phenylacrylamide (SUSM-528). Yield: 31% (37 mg from 100 mg); a grey solid; R_{f}=0.58 (MeOH:EtOAc, 0.5:9.5, v/v); ¹H NMR (600 MHz, CDCl₃): δ (ppm) 7.86 (s, 1H), 7.53 (d, J=7.6 Hz, 2H), 7.44 (s, 1H), 7.32 (t, J=7.5 Hz, 2H), 7.11 (t, J=7.4 Hz, 1H), 7.04 (s, 1H), 7.00 (t, J=7.9 Hz, 1H), 6.91 (d, J 7.5 Hz, 1H), 6.45 (d, J 7.7 Hz, 1H), 3.87 (s, 3H), 3.69 (s, 3H), 3.18-3.15 (m, 1H), 3.13-3.10 (m, 1H), 3.07-2.96 (m, 3H), 2.94-2.90 (m, 1H), 2.55-2.51 (m, 1H), 2.46-2.43 (m, 1H), 2.41-2.37 (m, 1H), 1.92 (d, J=12.9 Hz, 1H), 1.86-1.83 (m, 1H), 1.71-1.69 (m, 1H), 1.34-1.30 (m, 1H), 0.91 (t, J=7.4 Hz, 3H); ¹³C NMR (151 MHz, CDCl₃): δ (ppm)=167.7, 154.8, 154.6, 138.4, 137.4, 133.6, 129.1, 124.3, 121.9, 120.2, 117.7, 117.2, 107.9, 104.5, 99.8, 61.3, 61.2, 57.8, 55.4, 53.8, 40.9, 40.6, 30.2, 24.1, 19.6, 13.0. MS (ESI⁺): m/z=459.3 [M+H]⁺.

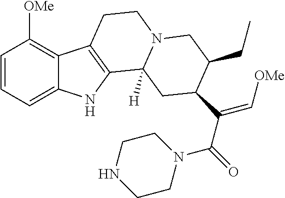 [SMILES: CC[C@@H]1CN2CCc3c([nH]c4cccc(OC)c34)[C@@H]2C[C@@H]1/C(=C\OC)C(=O)N1CCNCC1]

(E)-2-((2S,3S,12bS)-3-ethyl-8-methoxy-1,2,3,4,6,7,12,12b-octahydroindolo[2,3-a]quinolizin-2-yl)-3-methoxy-1-(piperazin-1-yl)prop-2-en-1-one (SUSM-562). ¹H NMR (600 MHz, CDCl₃): δ (ppm) 7.86 (s, 1H), 6.98 (t, J=7.9 Hz, 1H), 6.90 (d, J=7.7 Hz, 1H), 6.44 (d, J=8.1 Hz, 1H), 6.16 (s, 1H), 3.87 (s, 3H), 3.65-3.57 (m, 7H), 3.17 (d, J=11.1 Hz, 1H), 3.13-3.07 (m, 1H), 2.99-2.93 (m, 3H), 2.91-2.87 (m, 2H), 2.86-2.81 (m, 4H), 2.54-2.50 (m, 1H), 2.40-2.37 (m, 1H), 2.17-2.12 (m, 1H), 1.89-1.83 (m, 1H), 1.29-1.23 (m, 1H), 0.90 (t, J=7.4 Hz, 3H); ¹³C NMR (151 MHz, CDCl₃): δ (ppm)=172.6, 154.6, 148.2, 143.7, 137.4, 133.7, 121.9, 117.6, 108.9, 104.4, 99.8, 96.4, 88.2, 60.6, 55.5, 46.5, 40.5, 30.2, 22.8, 13.1. MS (ESI⁺): m/z=453.2 [M+H]⁺.

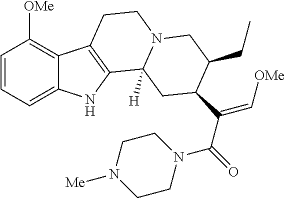 [SMILES: CC[C@@H]1CN2CCc3c([nH]c4cccc(OC)c34)[C@@H]2C[C@@H]1/C(=C\OC)C(=O)N1CCN(C)CC1]

(E)-2-((2S,3S,12bS)-3-ethyl-8-methoxy-1,2,3,4,6,7,12,12b-octahydroindolo[2,3-a]quinolizin-2-yl)-3-methoxy-1-(4-methylpiperazin-1-yl)prop-2-en-1-one (SUSM-561). ¹H NMR (600 MHz, CDCl₃): δ (ppm) 8.03 (s, 1H), 6.98 (t, J OMe=7.9 Hz, 1H), 6.90 (d, J=8.1 Hz, 1H), 6.43 (d, J=7.8 Hz, 1H), 6.18 (s, 1H), 3.86 (s, 3H), 3.71-3.63 (m, 4H), 3.60 (s, 3H), 3.14-3.07 (m, 2H), 2.98-2.94 (m, 2H), 2.90-2.88 (m, 2H), 2.53-2.49 (m, 1H), 2.41-2.35 (m, 5H), 2.30 (s, 3H), 2.17-2.10 (m, 1H), 2.01-1.99 (m, 1H), 1.89-1.85 (m, 1H), 1.63 (d, J=10.7 Hz, 1H), 1.29-1.24 (m, 1H), 0.89 (t, J=7.4 Hz, 3H); ¹³C NMR (151 MHz, CDCl₃): δ (ppm)=170.8, 154.5, 149.7, 137.4, 133.8, 121.8, 117.7, 115.2, 107.7, 104.5, 99.7, 77.4, 77.2, 76.9, 61.1, 60.6, 57.7, 55.4, 55.3, 53.8, 46.2, 41.9, 40.5, 31.7, 30.2, 24.1, 22.7, 19.5, 14.2, 13.0. MS (ESI⁺): m/z=467.3 [M+H]⁺.

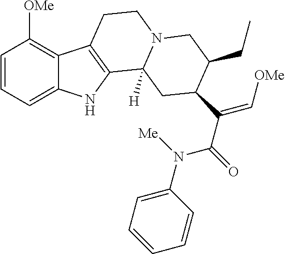 [SMILES: CC[C@@H]1CN2CCc3c([nH]c4cccc(OC)c34)[C@@H]2C[C@@H]1/C(=C\OC)C(=O)N(C)c1ccccc1]

(E)-2-((2S,3S,12bS)-3-ethyl-8-methoxy-1,2,3,4,6,7,12,12b-octahydroindolo[2,3-a]quinolizin-2-yl)-3-methoxy-N-methyl-N-phenylacrylamide (SUSM-529). ¹H NMR (600 MHz, CDCl₃): δ (ppm) 7.80 (s, 1H), 7.38 (t, J=7.9 Hz, 2H), 7.25 (d, J=7.3 Hz, 1H), 7.15 (d, J=7.3 Hz, 2H), 6.97 (t, J=7.9 Hz, 1H), 6.89 (d, J=7.7 Hz, 1H), 6.42 (d, J=7.4 Hz, 1H), 6.25 (s, 1H), 3.85 (s, 3H), 3.36 (s, 3H), 3.31 (s, 3H), 3.11-3.05 (m, 1H), 3.00-2.92 (m, 3H), 2.89-2.86 (m, 1H), 2.79-2.78 (m, 1H), 2.49-2.44 (m, 1H), 2.33-2.30 (m, 1H), 2.25-2.19 (m, 1H), 1.75-1.70 (m, 1H), 1.63 (dd, J=8.8, 5.8 Hz, 1H), 1.56-1.54 (m, 1H), 1.12-1.06 (m, 1H), 0.88 (t, J=7.3 Hz, 3H); ¹³C NMR (151 MHz, CDCl₃): δ (ppm)=172.2, 165.9, 156.0, 154.6, 146.3, 137.4, 133.9, 129.4, 127.0, 126.5, 121.8, 117.7, 115.2, 107.7, 104.4, 99.8, 61.3, 60.5, 57.7, 55.4, 53.8, 41.9, 40.7, 34.8, 31.7, 30.0, 25.4, 24.1, 22.8, 18.9, 14.2, 12.8. MS (ESI⁺): m/z=474.3 [M+H]⁺.

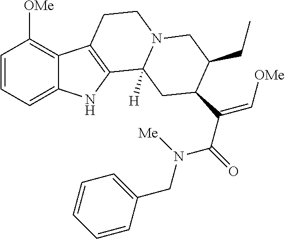 [SMILES: CC[C@@H]1CN2CCc3c([nH]c4cccc(OC)c34)[C@@H]2C[C@@H]1/C(=C\OC)C(=O)N(C)Cc1ccccc1]

(E)-N-benzyl-2-((2S,3S,12bS)-3-ethyl-8-methoxy-1,2,3,4,6,7,12,12b-octahydroindolo[2,3-a]quinolizin-2-yl)-3-methoxy-N-methylacrylamide (SUSM-563). ¹H NMR (600 MHz, CDCl₃): δ (ppm) 7.84 (s, 1H), 7.37-7.34 (m, 2H), 7.31-7.29 (m, 1H), 7.26-7.24 (m, 2H), 6.99 (t, J=7.9 Hz, 1H), 6.90 (d, J=8.0 Hz, 1H), 6.44 (d, J=7.7 Hz, 1H), 6.19 (s, 1H), 4.66 (d, J=5.0 Hz, 2H), 3.87 (s, 3H), 3.53 (s, 2H), 3.15-3.08 (m, 2H), 3.00-2.89 (m, 7H), 2.51 (td, J=11.5, 4.2 Hz, 1H), 2.39 (dd, J=11.4, 1.7 Hz, 1H), 2.17-2.11 (m, 1H), 1.96-1.94 (m, 1H), 1.88-1.82 (m, 2H), 1.77-1.74 (m, 1H), 1.37-1.29 (m, 2H), 0.88 (t, J=7.4 Hz, 3H); ¹³C NMR (151 MHz, CDCl₃): δ (ppm)=172.1, 165.9, 154.6, 149.1, 137.5, 137.4, 133.7, 128.9, 127.6, 121.9, 117.7, 115.5, 107.8, 104.4, 99.8, 61.1, 60.4, 57.7, 55.4, 53.9, 41.7, 40.5, 34.8, 31.7, 30.1, 25.4, 24.1, 22.8, 19.5, 13.0. MS (ESI⁺): m/z=488.4 [M+H]⁺.

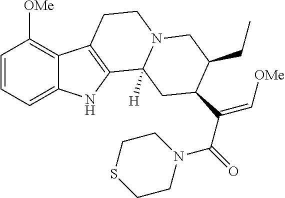 [SMILES: CC[C@@H]1CN2CCc3c([nH]c4cccc(OC)c34)[C@@H]2C[C@@H]1/C(=C\OC)C(=O)N1CCSCC1]

(E)-2-((2S,3S,12bS)-3-ethyl-8-methoxy-1,2,3,4,6,7,12,12b-octahydroindolo[2,3-a]quinolizin-2-yl)-3-methoxy-1-thiomorpholinoprop-2-en-1-one (SUSM-564). ¹H NMR (600 MHz, CDCl₃): δ (ppm) 7.77 (s, 1H), 6.99 (t, J=7.9 Hz, 1H), 6.90 (d, J=7.7 Hz, 1H), 6.45 (d, J=7.1 Hz, 1H), 6.19 (s, 1H), 3.90-3.88 (m, 4H), 3.87 (s, 3H), 3.61 (s, 3H), 3.18-3.16 (m, 1H), 3.12-3.07 (m, 1H), 3.00-2.94 (m, 2H), 2.91-2.85 (m, 2H), 2.66-2.58 (m, 5H), 2.53 (td, J=11.5, 4.2 Hz, 1H), 2.40-2.38 (m, 1H), 2.21-2.15 (m, 1H), 2.02-1.99 (m, 1H), 1.90-1.87 (m, 1H), 1.65 (d, J=11.3 Hz, 2H), 1.29-1.26 (m, 1H), 0.91 (t, J=7.4 Hz, 3H); ¹³C NMR (151 MHz, CDCl₃): δ (ppm)=171.2, 154.6, 149.6, 137.4, 133.6, 122.0, 117.7, 115.3, 108.0, 104.4, 99.9, 61.1, 60.7, 57.7, 55.5, 53.8, 42.0, 40.6, 30.3, 27.9, 24.1, 19.5, 13.0. MS (ESI⁺): m/z=470.2 [M+H]⁺.

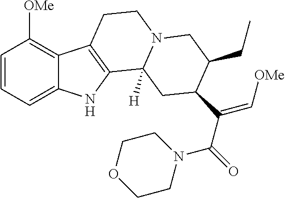 [SMILES: CC[C@@H]1CN2CCc3c([nH]c4cccc(OC)c34)[C@@H]2C[C@@H]1/C(=C\OC)C(=O)N1CCOCC1]

(E)-2-((2S,3S,12bS)-3-ethyl-8-methoxy-1,2,3,4,6,7,12,12b-octahydroindolo[2,3-a]quinolizin-2-yl)-3-methoxy-1-morpholinoprop-2-en-1-one (SUSM-527). ¹H NMR (600 MHz, CDCl₃): δ (ppm) 7.93 (s, 1H), 6.98 (t, J=7.9 Hz, 1H), 6.90 (d, J=8.0 Hz, 1H), 6.44 (d, J=7.7 Hz, 1H), 6.19 (s, 1H), 3.87 (s, 3H), 3.69-3.63 (m, 7H), 3.61-3.60 (m, 4H), 3.16-3.13 (m, 1H), 3.12-3.07 (m, 1H), 2.99-2.94 (m, 2H), 2.91-2.88 (m, 2H), 2.54-2.50 (m, 1H), 2.40-2.38 (m, 1H), 2.15 (td, J=12.9, 11.3 Hz, 1H), 2.01 (d, J=12.9 Hz, 1H), 1.91-1.85 (m, 1H), 1.64 (d, J=11.1 Hz, 1H), 1.26-1.22 (m, 1H), 0.89 (t, J=7.4 Hz, 3H); ¹³C NMR (151 MHz, CDCl₃): δ (ppm)=170.9, 154.6, 150.0, 137.4, 133.7, 121.9, 117.7, 114.9, 107.9, 104.4, 99.8, 67.1, 61.1, 60.7, 60.5, 57.7, 55.4, 53.8, 41.8, 40.6, 38.7, 30.2, 24.1, 21.2, 19.5, 14.3, 13.0. MS (ESI⁺): m/z=454.3 [M+H]⁺.

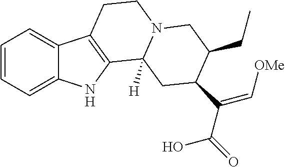 [SMILES: CC[C@@H]1CN2CCc3c([nH]c4ccccc34)[C@@H]2C[C@@H]1/C(=C\OC)C(=O)O]

(E)-2-((2S,3S,12bS)-3-ethyl-1,2,3,4,6,7,12,12b-octahydroindolo[2,3-a]quinolizin-2-yl)-3-methoxyacrylic acid (SUSM-572). 1H NMR (600 MHz, CD₃OD): δ (ppm) 7.42 (s, 1H), 7.40 (d, J=8.0 Hz, 1H), 7.31 (d, J=8.1 Hz, 1H), 7.06 (t, J=7.4 Hz, 1H), 6.99 (t, J=7.4 Hz, 1H), 3.71 (s, 3H), 3.65 (d, J=11.8 Hz, 1H), 3.27-3.20 (m, 3H), 3.11-3.07 (m, 1H), 2.87-2.76 (m, 3H), 2.54-2.48 (m, 1H), 2.31-2.28 (m, 1H), 1.87-1.83 (m, 1H), 1.70-1.64 (m, 1H), 1.47-1.42 (m, 1H), 0.91 (t, J=7.4 Hz, 3H); ¹³C NMR (151 MHz, CD₃OD): δ (ppm)=173.6, 159.0, 136.8, 126.7, 120.9, 118.5, 117.3, 113.6, 110.7, 105.9, 60.1, 56.9, 53.2, 39.7, 29.3, 28.9, 20.6, 20.2, 19.1, 14.3, 11.7. MS (ESI⁺): m/z=355.2 [M+H]⁺.

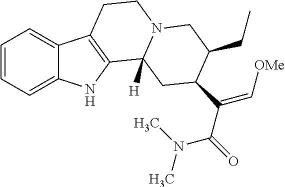 [SMILES: CC[C@@H]1CN2CCc3c([nH]c4ccccc34)[C@H]2C[C@@H]1/C(=C\OC)C(=O)N(C)C]

(E)-2-((2S,3S,12bR)-3-ethyl-1,2,3,4,6,7,12,12b-octahydroindolo[2,3-a]quinolizin-2-yl)-3-methoxy-N,N-bis(methyl-d3)acrylamide (SUSM-573). ¹H NMR (600 MHz, CDCl₃): δ (ppm) 7.90 (s, 1H), 7.46 (d, J=7.6 Hz, 1H), 7.30 (d, J=7.9 Hz, 1H), 7.11 (td, J=8.0, 1.3 Hz, 2H), 7.06 (td, J=8.0, 1.1 Hz, 1H), 6.18 (s, 1H), 3.60 (s, 3H), 3.21-3.19 (m, 1H), 3.01-2.91 (m, 4H), 2.70-2.67 (m, 1H), 2.58-2.54 (m, 1H), 2.43-2.41 (m, 1H), 2.22-2.15 (m, 1H), 2.06-2.03 (m, 1H), 1.89-1.84 (m, 1H), 1.71-1.67 (m, 1H), 1.34-1.25 (m, 1H), 0.91 (t, J=7.5 Hz, 3H); ¹³C NMR (151 MHz, CDCl₃): δ (ppm)=172.0, 149.6, 136.1, 135.7, 127.6, 121.2, 119.3, 118.1, 115.5, 110.9, 108.1, 61.1, 60.5, 57.7, 53.5, 41.9, 40.5, 31.7, 30.2, 25.4, 22.8, 22.0, 19.5, 14.3. MS (ESI⁺): m/z=388.3 [M+H]⁺.

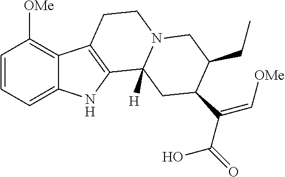 [SMILES: CC[C@@H]1CN2CCc3c([nH]c4cccc(OC)c34)[C@H]2C[C@@H]1/C(=C\OC)C(=O)O]

(E)-2-((2S,3S,12bR)-3-ethyl-8-methoxy-1,2,3,4,6,7,12,12b-octahydroindolo[2,3-a]quinolizin-2-yl)-3-methoxyacrylic acid (SUSM-574). ¹H NMR (600 MHz, CD₃OD): δ (ppm) 7.32 (s, 1H), 6.99 (t, J=7.9 Hz, 1H), 6.91 (d, J=8.2 Hz, 1H), 6.46 (d, J=7.7 Hz, 1H), 3.84 (s, 3H), 3.76 (s, 3H), 3.45-3.30 (m, 4H), 3.11-2.90 (m, 2H), 2.80-2.75 (m, 1H), 2.56-2.25 (m, 4H), 1.56-1.51 (m, 1H), 1.19-1.12 (m, 1H), 0.88 (t, J=7.5 Hz, 3H); ¹³C NMR (151 MHz, CD₃OD): δ (ppm)=159.5, 155.7, 139.7, 123.8, 117.8, 115.1, 106.8, 105.9, 100.4, 62.4, 61.5, 55.5, 54.1, 39.1, 33.2, 30.7, 24.9, 22.9, 22.1, 15.8, 11.1. MS (ESI⁺): m/z=385.3 [M+H]⁺.

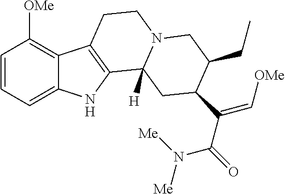 [SMILES: CC[C@@H]1CN2CCc3c([nH]c4cccc(OC)c34)[C@H]2C[C@@H]1/C(=C\OC)C(=O)N(C)C]

(E)-2-((2S,3S,12bR)-3-ethyl-8-methoxy-1,2,3,4,6,7,12,12b-octahydroindolo[2,3-a]quinolizin-2-yl)-3-methoxy-N,N-dimethylacrylamide (SUSM-575). ¹H NMR (600 MHz, CDCl₃): δ (ppm) 7.97 (s, 1H), 6.98 (t, J=8.1 Hz, 1H), 6.88 (d, J=7.0 Hz, 1H), 6.44 (d, J=8.0 Hz, 1H), 6.23 (s, 1H), 3.86 (s, 3H), 3.63 (s, 3H), 3.19-3.13 (m, 3H), 3.04-2.96 (m, 8H), 2.56-2.52 (m, 1H), 2.40-2.35 (m, 1H), 2.19-2.12 (m, 2H), 2.04-1.94 (m, 3H), 1.64-1.60 (m, 1H), 1.13-1.08 (m, 1H), 0.89 (d, J=7.5 Hz, 3H); ¹³C NMR (151 MHz, CDCl₃): δ (ppm)=171.7, 154.6, 150.2, 137.5, 133.3, 121.8, 117.6, 115.2, 107.8, 104.4, 99.8, 60.9, 60.5, 60.3, 55.4, 53.6, 42.1, 39.7, 34.8, 31.0, 24.8, 23.9, 21.2, 14.3, 11.4. MS (ESI⁺): m/z=412.2 [M+H]⁺.

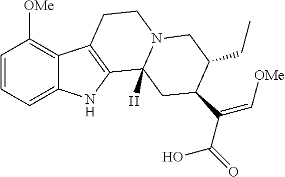 [SMILES: CC[C@H]1CN2CCc3c([nH]c4cccc(OC)c34)[C@H]2C[C@@H]1/C(=C\OC)C(=O)O]

(E)-2-((2S,3R,12bR)-3-ethyl-8-methoxy-1,2,3,4,6,7,12,12b-octahydroindolo[2,3-a]quinolizin-2-yl)-3-methoxyacrylic acid (SUSM-576). ¹H NMR (600 MHz, CD₃OD): δ (ppm) 7.44 (s, 1H), 7.02 (t, J=8.0 Hz, 1H), 6.90 (d, J=8.2 Hz, 1H), 6.48 (d, J=7.8 Hz, 1H), 5.09 (d, J=11.0 Hz, 1H), 3.86 (s, 3H), 3.79-3.73 (m, 4H), 3.53-3.40 (m, 2H), 3.34-3.28 (m, 2H), 3.20-3.15 (m, 2H), 2.62-2.43 (m, 1H), 2.09-2.04 (s, 2H), 1.40-1.36 (m, 2H), 0.92 (t, J=7.4 Hz, 3H); ¹³C NMR (151 MHz, CD₃OD): δ (ppm)=174.9, 159.9, 155.7, 139.8, 124.2, 117.7, 114.0, 106.6, 105.9, 100.5, 61.6, 55.6, 47.8, 43.5, 38.9, 30.7, 15.8, 12.1, 11.6. MS (ESI⁺): m/z=385.3 [M+H]⁺.

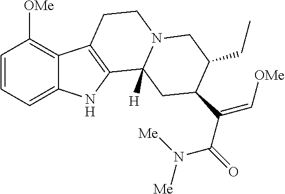 [SMILES: CC[C@H]1CN2CCc3c([nH]c4cccc(OC)c34)[C@H]2C[C@@H]1/C(=C\OC)C(=O)N(C)C]

(E)-2-((2S,3R,12bR)-3-ethyl-8-methoxy-1,2,3,4,6,7,12,12b-octahydroindolo[2,3-a]quinolizin-2-yl)-3-methoxy-N,N-dimethylacrylamide (SUSM-577). ¹H NMR (600 MHz, CDCl₃): δ (ppm) 7.99 (s, 1H), 6.99 (t, J=7.9 Hz, 1H), 6.90 (d, J=8.4 Hz, 1H), 6.45 (d, J=7.2 Hz, 1H), 6.10 (s, 1H), 3.87 (s, 3H), 3.64 (s, 3H), 3.19-3.13 (m, 1H), 3.08-2.99 (m, 8H), 2.93-2.89 (m, 1H), 2.82 (dd, J=11.4, 3.8 Hz, 1H), 2.57-2.54 (m, 1H), 2.45-2.41 (m, 1H), 2.01-1.96 (m, 3H), 1.85 (s, 1H), 1.55 (s, 1H), 1.46-1.43 (m, 1H), 0.94 (t, J=7.4 Hz, 3H); ¹³C NMR (151 MHz, CDCl₃): δ (ppm)=171.9, 154.5, 148.9, 137.4, 121.8, 117.9, 114.8, 108.0, 104.5, 99.8, 60.2, 55.4, 53.0, 35.3, 31.7, 31.1, 29.8, 29.4, 22.8, 14.2, 12.7. MS (ESI⁺): m/z=412.2 [M+H]⁺.

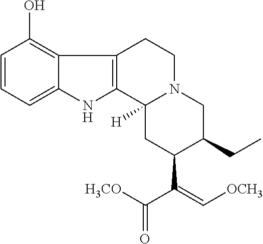 [SMILES: CC[C@@H]1CN2CCc3c([nH]c4cccc(O)c34)[C@@H]2C[C@@H]1/C(=C\OC)C(=O)OC]

Methyl (E)-2-((2S,3S,12bS)-3-ethyl-8-hydroxy-1,2,3,4,6,7,12,12b-octahydroindolo[2,3-a]quinolizin-2-yl)-3-methoxyacrylate. (9-OH Corynantheidine)¹H NMR (600 MHz, CDCl₃) δ 7.77 (s, 1H), 7.43 (s, 1H), 6.90 (t, J=7.8 Hz, 1H), 6.86 (d, J=8.0 Hz, 1H), 6.39 (d, J=7.5 Hz, 1H), 5.22 (s, 1H), 3.72 (s, 3H), 3.71 (s, 3H), 3.23-3.14 (m, 2H), 3.03 (ddd, J=13.7, 9.2, 3.0 Hz, 2H), 2.98-2.91 (m, 2H), 2.58-2.45 (m, 3H), 1.80-1.75 (m, 2H), 1.65-1.62 (m, 1H), 1.23-1.18 (m, 1H), 0.87 (t, J=7.4 Hz, 3H). ¹³C NMR (151 MHz, CDCl₃) δ (ppm)=169.4, 160.7, 150.1, 138.0, 134.2, 122.1, 116.8, 111.6, 106.9, 104.4, 103.9, 61.7, 61.4, 57.8, 53.7, 51.5, 40.8, 40.0, 30.0, 23.8, 19.2, 12.9. MS (ESI⁺): m/z=385.2 [M+H]⁺.

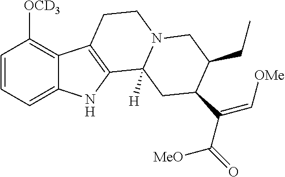

Methyl (E)-2-((2S,3S,12bS)-3-ethyl-8-(methoxy-d3)-1,2,3,4,6,7,12,12b-octahydroindolo[2,3-a]quinolizin-2-yl)-3-methoxyacrylate (SUSM-567). ¹H NMR (600 MHz, CDCl₃): δ (ppm) 7.74 (s, 1H), 7.43 (s, 1H), 6.99 (t, J=7.9 Hz, 1H), 6.90 (d, J=8.0 Hz, 1H), 6.45 (d, J=7.7 Hz, 1H), 3.72 (s, 3H), 3.71 (s, 3H), 3.16-3.10 (m, 2H), 3.05-2.98 (m, 3H), 2.94-2.91 (m, 1H), 2.55-2.49 (m, 2H), 2.47-2.44 (m, 1H), 1.81-1.78 (m, 2H), 1.63-1.62 (m, 1H), 0.87 (t, J=7.4 Hz, 3H); ¹³C NMR (151 MHz, CDCl₃): δ (ppm)=169.4, 160.7, 154.6, 137.4, 133.8, 121.9, 117.8, 111.7, 107.9, 104.3, 99.9, 61.7, 61.4, 57.9, 53.9, 51.5, 40.8, 40.1, 24.1, 22.8, 19.2, 14.3, 13.0. MS (ESI⁺): m/z=402.2 [M+H]⁺.

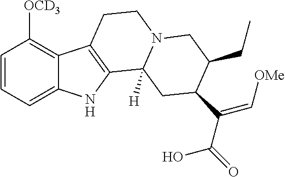

(E)-2-((2S,3S,12bS)-3-ethyl-8-(methoxy-d3)-1,2,3,4,6,7,12,12b-octahydroindolo[2,3-a]quinolizin-2-yl)-3-methoxyacrylic acid (SUSM-568). ¹H NMR (600 MHz, CD₃OD): δ (ppm) 7.37 (s, 1H), 6.94-6.88 (m, 2H), 6.40 (d, J=7.6 Hz, 1H), 5.92-5.78 (m, 2H), 3.58 (s, 3H), 3.18-3.08 (m, 3H), 2.96-2.94 (m, 3H), 2.52-2.38 (m, 3H), 1.85-1.62 (m, 2H), 1.32-1.28 (m, 1H), 0.84 (t, J=7.0 Hz, 3H); ¹³C NMR (151 MHz, CD₃OD): δ (ppm)=173.8, 173.3, 158.7, 154.5, 137.7, 134.4, 121.8, 117.5, 114.0, 106.3, 104.8, 99.6, 61.0, 57.5, 55.0, 50.8, 41.2, 31.7, 28.8, 24.0, 22.7, 14.2, 12.9. MS (ESI⁺): m/z=388.2 [M+H]⁺.

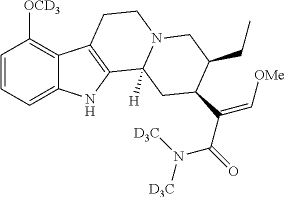

(E)-2-((2S,3S,12bS)-3-ethyl-8-(methoxy-d3)-1,2,3,4,6,7,12,12b-octahydroindolo[2,3-a]quinolizin-2-yl)-3-methoxy-N,N-bis(methyl-d3)acrylamide (SUSM-570). ¹H NMR (600 MHz, CDCl₃): δ (ppm) 7.89 (s, 1H), 6.98 (t, J=7.9 Hz, 1H), 6.90 (d, J=8.1 Hz, 1H), 6.43 (d, J=7.7 Hz, 1H), 6.17 (s, 1H), 3.60 (s, 3H), 3.17-3.14 (m, 2H), 3.13-3.08 (m, 1H), 3.00-2.94 (m, 2H), 2.91-2.88 (m, 2H), 2.54-2.49 (m, 1H), 2.39-2.37 (m, 1H), 2.18-2.12 (m, 1H), 2.02-2.00 (m, 1H), 1.88-1.84 (m, 1H), 1.68-1.66 (m, 1H), 1.33-1.28 (m, 1H), 0.91 (t, J=7.4 Hz, 3H); ¹³C NMR (151 MHz, CDCl₃): δ (ppm)=172.1, 154.6, 149.6, 137.4, 133.8, 121.8, 117.7, 115.6, 107.8, 104.4, 99.8, 61.1, 60.5, 57.7, 53.8, 41.8, 40.5, 34.8, 31.7, 30.2, 25.4, 24.1, 22.8, 19.5, 13.1. MS (ESI⁺): m/z=421.3 [M+H]⁺.

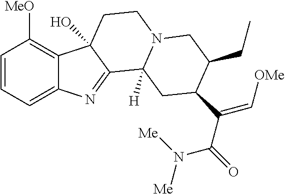 [SMILES: CC[C@@H]1CN2CC[C@@]3(O)C(=Nc4cccc(OC)c43)[C@@H]2C[C@@H]1/C(=C\OC)C(=O)N(C)C]

(E)-2-((2S,3S,7aS,12bS)-3-ethyl-7a-hydroxy-8-methoxy-1,2,3,4,6,7,7a,12b-octahydroindolo[2,3-a]quinolizin-2-yl)-3-methoxy-N,N-dimethylacrylamide (SUSM-524). Yield: 32% (35 mg from 100 mg); a white solid; R_{f}=0.31 (MeOH:EtOAc, 0.5:9.5, v/v); ¹H NMR (600 MHz, CDCl₃): δ (ppm) 7.31-7.28 (m, 1H), 7.20 (d, J=7.6 Hz, 1H), 6.74 (d, J=8.3 Hz, 1H), 6.17 (s, 1H), 3.88 (s, 3H), 3.66 (s, 3H), 3.15 (dd, J=11.1, 2.6 Hz, 1H), 3.04 (s, 1H), 3.01 (s, 6H), 2.82-2.76 (m, 2H), 2.65-2.61 (m, 2H), 2.56-2.50 (m, 1H), 2.43-2.41 (m, 1H), 2.05-2.03 (m, 1H), 1.76-1.74 (m, 2H), 1.69-1.64 (m, 1H), 1.44-1.40 (m, 1H), 1.32-1.26 (m, 1H), 0.87 (t, J=6.0 Hz, 3H); ¹³C NMR (151 MHz, CDCl₃): δ (ppm)=184.2, 172.1, 156.0, 155.2, 149.7, 130.9, 126.7, 115.4, 114.4, 109.0, 81.2, 77.4, 77.2, 76.9, 61.3, 60.6, 58.2, 55.6, 50.1, 41.4, 40.1, 36.1, 31.7, 26.3, 22.8, 19.2, 14.2, 13.0. MS (ESI⁺): m/z=428.3 [M+H]⁺.

### Metabolic Stability of Kratom Alkaloids in Rat Liver Microsomes

In vitro metabolic stability of each compound was determined using rat liver microsomes in triplicate. Verapamil was used as a positive control to check the activity of the microsomes. The incubation mixtures consisted of rat liver microsomes (1 mg microsomal protein/ml), substrate (1 μM), and reduced NADPH (1 mM) in a total volume of 0.4 ml potassium phosphate buffer (50 mM, pH 7.4). Reactions were initiated with the addition of NADPH and kept in an incubator shaker at 37° C. Aliquots of 25 μl were withdrawn at 0, 5, 10, 15, 30, and 60 min and mixed with 125 μl of acetonitrile with formic acid (0.1% v/v) containing phenacetin (50 ng/ml; internal standard) for the termination of the reaction. The samples were then vortex mixed and filtered through a 0.45 m 96-well membrane filtration plate under centrifugation at speed of 2000×g for 5 min at 4° C. The filtrates were subjected to UPLC-mass spectrometry (MS/MS) analysis.

The intrinsic clearance of the compounds was calculated using a half-life employing the ‘substrate depletion’ approach. The apparent half-life was calculated from the pseudo-first-order rate constants obtained by linear regression of log (concentration) and time plots. The in vitro intrinsic clearance of IFN was estimated using the formula:

$${t}_{1 / 2} = ln ( 2 ) / k$$

$$C {l}_{i n t} ( μ L / min / mg protein ) = \frac{0.693}{{t}_{1 / 2}} \times \frac{{V}_{i n c u b a tion i n μ L}}{Protein Concentration in mg}$$

Where k is the slope of the line obtained by plotting the natural logarithmic of the percentage of parent remaining versus time and V is the volume of incubation.

The in vitro intrinsic clearance was scaled to whole-organ (hepatic) in vivo intrinsic CL (CL_{int,H}) using the scaling factors available in the literature using equation (Mahmood I. Application of allometric principles for the prediction of pharmacokinetics in human and veterinary drug development. Advanced drug delivery reviews, 2007, 59(11), 1177-1192)

$$C {L}_{i n t , H} ( mL / min * kg ) = \frac{0.693}{{t}_{1 / 2} ( min )} \times \frac{Volume of incubation ( mL )}{Microsomal protein ( mg )} \times \frac{Liver weight ( g )}{Body weight ( kg )} \times MPPGL$$

MPPGL represents the microsomal protein per gram of Sprague Dawley rat liver (45 mg/g) and liver weight per kg body weight was 40 g/kg.

### Radioligand Binding Assay

Mitragynine amides were screened at Eurofins Cerep (Celle l'Evescault, France) for in vitro binding affinity at alpha adrenergic and opioid receptors. Briefly, each cell membrane homogenate was incubated with a radioligand in the absence or presence of the kratom alkaloids in a buffer. Nonspecific binding was determined in the presence of a specific agonist or antagonist at the target receptor. Following incubation, the samples were filtered rapidly under vacuum through glass fiber filters presoaked in a buffer and rinsed several times with an ice-cold buffer using a 48- or 96-sample cell harvester. The experimental conditions that were used for the assays are summarized in Table A2. The filters were counted for radioactivity in a scintillation counter using a scintillation cocktail. The IC₅₀ values (concentration causing a half-maximal inhibition of control specific binding) from the displacement data were computed using a nonlinear, least squares regression analysis (Prism; GraphPad Software, Inc., San Diego, CA) as well as the concentration of radioligand used in the assay, and the Eurofins Cerep's historical value for the K_{D} values of the radioligand. The IC₅₀ values converted to Ki values using the Cheng-Prusoff equation.

TABLE A2
Summary of scintillation counting conditions employed for assessing affinity at
various binding sites for mitragynine amides in competition for the specified
radioligand. These details were extracted from reports from Eurofins Scientific.

| Receptors | Source | Ligand | Concentration | | Ligand Kd | | Non-Specific | Incubation |
|---|---|---|---|---|---|---|---|---|
| DOP (h) | human | [3H]DADLE | 0.5 | nM | 0.6 | nM | naltrexone | 60 min RT |
| | recombinant | | | | | | (10 μM) | |
| | (Chem-1 | | | | | | | |
| | (RBL) cells) | | | | | | | |
| KOP (h) | human | [3H]U69,593 | 0.5 | nM | 0.6 | nM | naloxone | 60 min RT |
| | recombinant | | | | | | (10 μM) | |
| | (RBL cells) | | | | | | | |
| MOP (h) | human | [3H]DAMGO | 0.5 | nM | 0.35 | nM | naloxone | 120 min RT |
| | recombinant | | | | | | (10 μM) | |
| | (HEK-293 | | | | | | | |
| | cells) | | | | | | | |
| α2A (h) | human | [3H]RX | 1 | nM | 0.8 | nM | (−)epinephrine | 60 min RT |
| | recombinant | 821002 | | | | | (100 μM) | |
| | (CHO cells) | | | | | | | |
| α2C (h) | human | [3H]RX | 2 | nM | 0.95 | nM | (−)epinephrine | 60 min RT |
| | recombinant | 821002 | | | | | (100 μM) | |
| | (CHO cells) | | | | | | | |

Evaluation of Mitragynine Amide in cAMP Assay

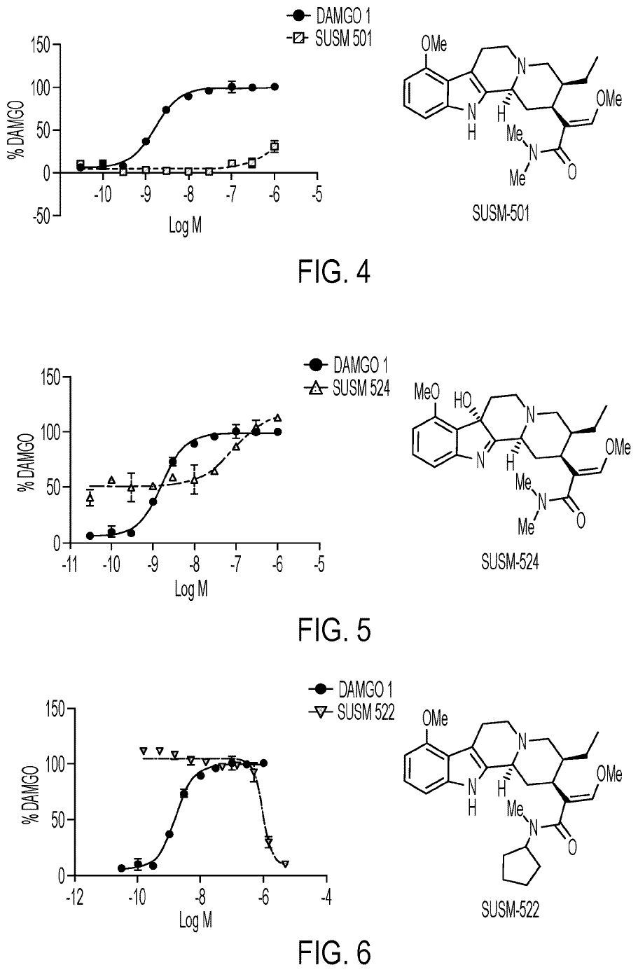

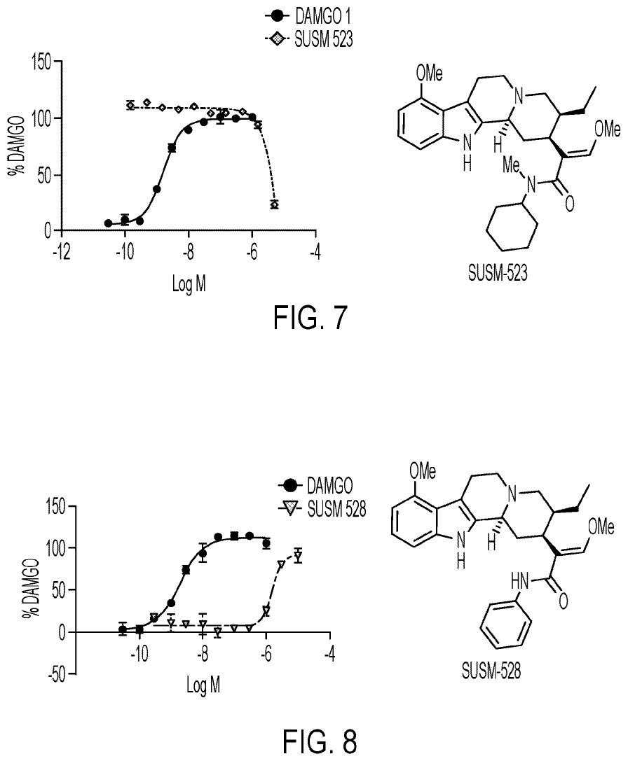

Compounds SUSM-501, SUSM-524, SUSM-522, SUSM-523, and SUSM-528 were evaluated for cyclic adenosine 3′,5′-monophosphate (cAMP) assay (described below). The cAMP functional assay was performed in N=1. Compounds SUSM-501 and SUSM-524 were investigated and it was found that the di-methylated mitragynine amide SUSM-501 produced partial agonist activity, while the 7-OH version of this molecule, SUSM-524, exhibited agonist activity. Compounds SUSM-522 and SUSM-523 acted as antagonists. Compound SUSM-528 acted as agonist, however its potency was less than DAMGO. The resulting functional activity of the individual compounds are shown in FIGS. 4-8.

Protocol for Cyclic Adenosine 3′,5′-Monophosphate (cAMP) Assay
Cells: CHO-MOR
Compounds tested: SUSM 501, 522, 523, 524 and 528.
Agonist used: DAMGO

Preparation of cell suspension: hMOR-CHO cell lines were maintained in DMEM/F₁₂ medium supplemented with 1% FBS, 100 units penicillin/streptomycin, and 50 mg/mL G418. Cells were incubated at 37° C. and under 5% C₀₂. Once they reached 60-80% confluence, they were scraped and detached from the 182 cm² flask and spun at 130 g for 10 minutes at 20° C. Freshly harvested cells were counted using a hemocytometer and used in the assay accordingly.

Dilutions and Assay: LANCE Ultra cAMP kit (RF0263) was used to detect cAMP accumulation in a white 96-well Optiplate. Stimulation buffer containing 1×HBSS, 1 M HEPES (pH 7.4), 7.5% BSA stabilizer, and 50 mM RO-20-1724 (Sigma, 557502) in DMSO was prepared. cAMP standard serial dilutions in stimulation buffer, as well as Forskolin (adenylyl cyclase activator), were done following the table provided in the kit's manual. Serial dilutions of the control agonist DAMGO were done to titrate from 1 μM to 30 μM in a stimulation buffer. Compounds were prepared as a stock solution of 10 mM in DMSO and then diluted to the working concentration range in the stimulation buffer. The cell pellet was resuspended in the previously prepared stimulation buffer and 5,000 cells (10 μL) were added to each well. After incubating for 30 min at room temperature to equilibrate the reaction, Eu-cAMP tracer, and uLIGHT-anti-cAMP working solutions were added per the manufacturer's instructions. The 96-well plate was centrifuged at 1,000 rpm, for 60 seconds at 20° C. to homogenize all reagents and incubated for 1 h. After incubation, the TR-FRET signal (ex 337 nm) was read on a CLARIO star multimode plate reader (BMG Biotech, Cary NC). Fluorescence values at 665 nm were plotted against the log of compound concentration and nonlinear regression analysis (4-parameters) was used to generate EC₅₀ values (GraphPad Prism, GraphPad Software, Inc., San Diego CA).

The optimal conditions for the assay such as cell density, assay window, and forskolin concentration were evaluated before testing compounds. For hMOR-CHO forskolin concentrations were 630 nM, and the cell density for was 5,000 cells/well.

### Compounds of Formulas (III), (IV), or (V)

In the present study, the analogues were synthesized via a multistep route which is outlined in Scheme 1. The synthesis was initiated with the Grignard reaction between 1-benzyl 4-piperidone and different phenylmagnesium bromides to get compound 1. Then the tertiary alcohol product 1 was treated with Pd/C under hydrogen atmosphere in EtOH to give debenzylated compound 2. Compound 2 was then treated with 1-bromo-3-butyne in presence of base K₂CO₃ to afford N-(3-butynyl)piperidine compound 3. One-pot sequential m-CPBA oxidation of compound 3 and gold-catalysis with Ph₃PAuNTf₂ at 0° C. indeed led to an excellent yield of bicyclic piperidin-4-one 4. Compound 4 then underwent Horner-Wadsworth-Emmons reaction with trimethylphosphonocaetate in presence of base NaH to give intermediate α, β-unsaturated ester 5. Hydrogenation of 5 with Pd/C under hydrogen atmosphere in MeOH provided saturated compound 6. When compound 6 was treated with trifluoroacetic acid, concomitant alcohol dehydration reaction took place to afford 7a and 7b. Again upon hydrogenation with Pd/C and hydrogen compound 7 gave saturated compound 8. Formylation of 8 with LDA and methyl formate provided intermediate 9. Subjecting 9 to dimethyl sulfate (as the methylating agent) in the presence of potassium carbonate provided the desired β-methylacrylate containing final compound 10.

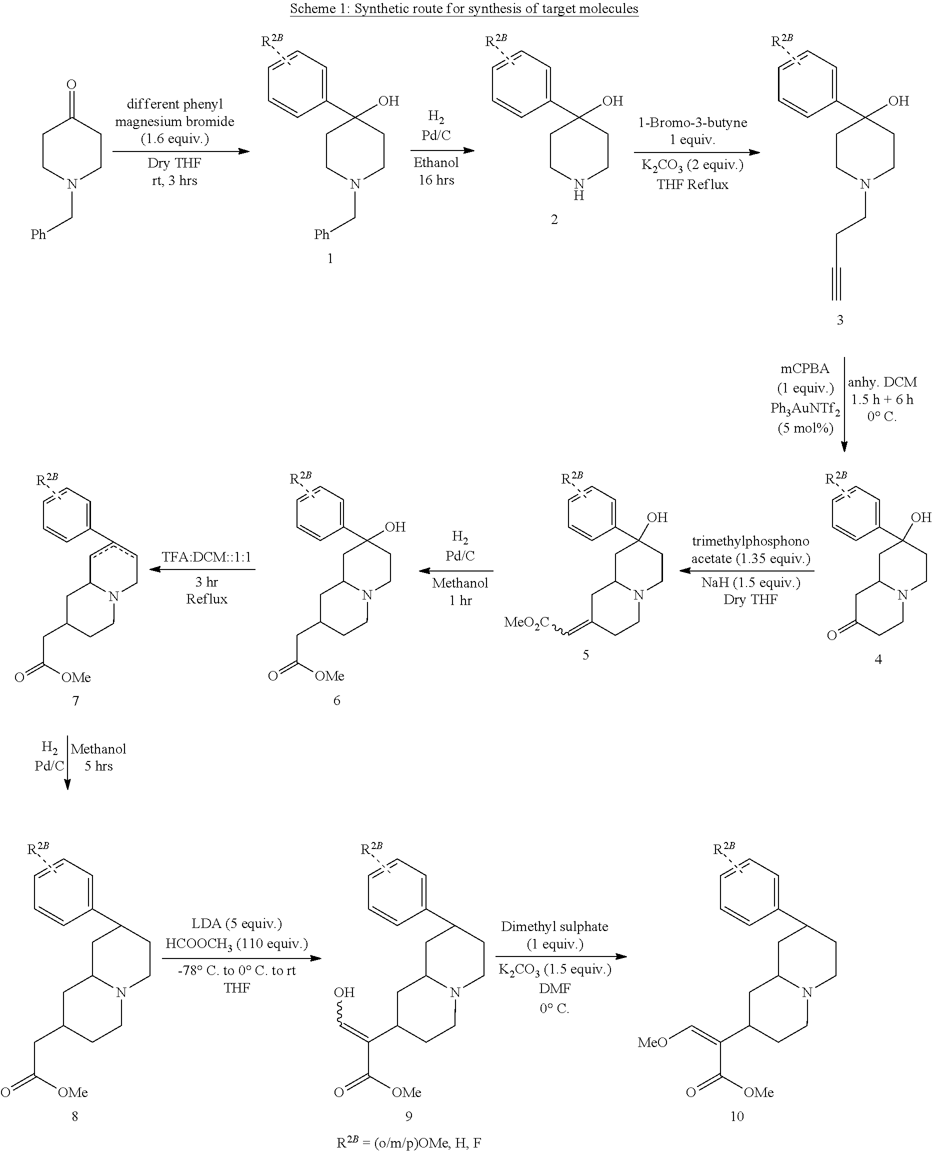

While synthesizing the mitragynine analogs, the intermediates were screened against MOR and α 2A. Piperidine derivative (SG-01-003) was tested against both MOR and a 2A and we found this compound was not selective towards both receptors. After cyclizing the piperidine ring into quinolizine ring, the affinity increased a significant amount towards α 2A and α-2C ARs. This trend was almost same up to the final compound except the MOR affinity regained after installing the β-methyl acrylate moiety to the ester derivative which is shown in table Bi1.

TABLE B1
Binding affinities of synthesised mitragynine analogs at opioid and α2-
adrenergic receptor subtype

| | | MOR | Alpha2A | |
|---|---|---|---|---|
| | | Ki ± SEM | Ki ± SEM | Alpha2C |
| Structure | Code | (nM) | (nM) | Ki ± SEM (nM) |
| 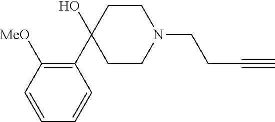 [SMILES: C#CCCN1CCC(O)(c2ccccc2OC)CC1] | SG-01-003 | >10,000 | 5754 ± 1174 | >10,000 |
| 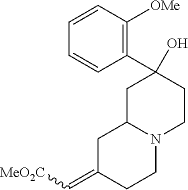 [SMILES: COC(=O)C=C1CCN2CCC(O)(c3ccccc3OC)CC2C1] | SG-01-005 | >10,000 | 264 ± 16.0 | 129 ± 37.90 |
| 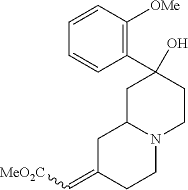 [SMILES: COC(=O)C=C1CCN2CCC(O)(c3ccccc3OC)CC2C1] | SG-01-005A | >10,000 | 1004 ± 254 | 489 ± 83.4 |
| 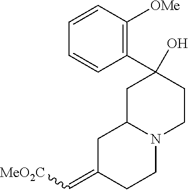 [SMILES: COC(=O)C=C1CCN2CCC(O)(c3ccccc3OC)CC2C1] | SG-01-005B | >10,000 | 110 ± 8 | 46.7 ± 4.04 |
| 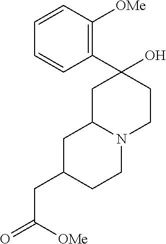 [SMILES: COC(=O)CC1CCN2CCC(O)(c3ccccc3OC)CC2C1] | SG-01-006 | >10,000 | 376 ± 72.2 | 139 ± 20.3 |
| 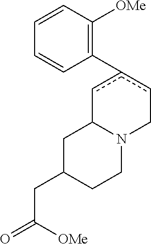 | SG-01-007A | >10,000 | 36.1 ± 2.64 | 22.3 ± 4.48 |
| 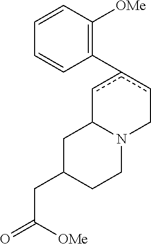 | SG-01-007B | >10,000 | 15.0 ± 0.369 | 12.2 ± 2.51 |
| 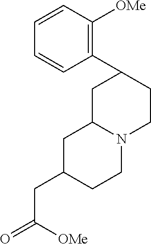 [SMILES: COC(=O)CC1CCN2CCC(c3ccccc3OC)CC2C1] | SG-01-008 | >10,000 | 27.9 ± 3.05 | 13.6 ± 0.828 |
| 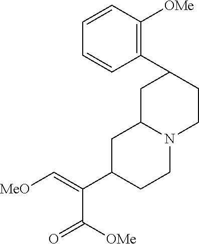 [SMILES: CO/C=C(\C(=O)OC)C1CCN2CCC(c3ccccc3OC)CC2C1] | SG-01-040 | SG-01-040 | 5992 ± 2883 | 145 ± 21 |

| | | | |
|---|---|---|---|
| Mitragynine | 1530 ± 143 | 709 ± 91.2 | 4090 ± 273 |

The values represent the mean ± SEM from 3 independent experiments. (*). NT: Not tested

TABLE B1′
Binding affinities of synthesised mitragynine analogs at opioid and
α2-adrenergic receptor subtype

| | MOR | KOR | DOR | |
|---|---|---|---|---|
| | Kᵢ ± | Kᵢ ± | Kᵢ ± | alpha2A |
| | SEM | SEM | SEM | Kᵢ ± SEM |
| Compound | (nM) | (nM) | (nM) | (nM) |
| 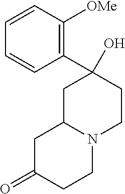 [SMILES: COc1ccccc1C1(O)CCN2CCC(=O)CC2C1] | >10000 | >10000 | >10000 | 5754 ± 1174 |
| 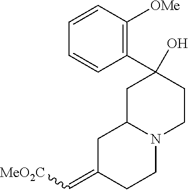 [SMILES: COC(=O)C=C1CCN2CCC(O)(c3ccccc3OC)CC2C1] | >10000 | >10000 | 2943 ± 2548 | 227 ± 37 |
| 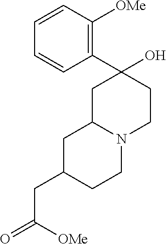 [SMILES: COC(=O)CC1CCN2CCC(O)(c3ccccc3OC)CC2C1] | >10000 | >10000 | >10000 | 3472 ± 1795 |
| 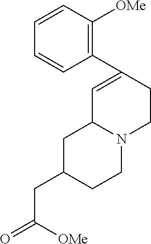 [SMILES: COC(=O)CC1CCN2CCC(c3ccccc3OC)=CC2C1] | >10000 | >10000 | >10000 | 39 ± 3 |
| 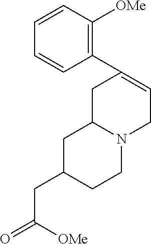 [SMILES: COC(=O)CC1CCN2CC=C(c3ccccc3OC)CC2C1] | >10000 | >10000 | >10000 | 15 ± 0 |
| 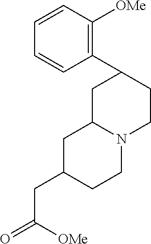 [SMILES: COC(=O)CC1CCN2CCC(c3ccccc3OC)CC2C1] | >10000 | >10000 | >10000 | 24 ± 3 |
| 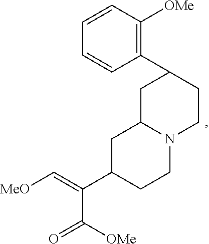 [SMILES: CO/C=C(\C(=O)OC)C1CCN2CCC(c3ccccc3OC)CC2C1] | 4226 ± 736 | 1024 ± 118 | >10000 | 125 ± 21 |
| 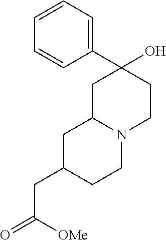 [SMILES: COC(=O)CC1CCN2CCC(O)(c3ccccc3)CC2C1] | 8614 ± 2528 | 3602 ± 1084 | >10000 | 129 ± 49 |
| 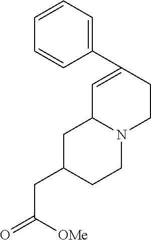 [SMILES: COC(=O)CC1CCN2CCC(c3ccccc3)=CC2C1] | 7929 ± 1343 | 683 ± 142 | >10000 | 5 ± 2 |
| 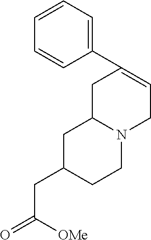 [SMILES: COC(=O)CC1CCN2CC=C(c3ccccc3)CC2C1] | 6981 ± 1452 | 957 ± 188 | >10000 | 7 ± 2 |
| 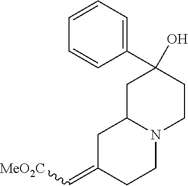 [SMILES: COC(=O)C=C1CCN2CCC(O)(c3ccccc3)CC2C1] | >10000 | 7655 ± 2671 | >10000 | 155 ± 65 |
| 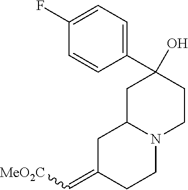 [SMILES: COC(=O)C=C1CCN2CCC(O)(c3ccc(F)cc3)CC2C1] | 672 ± 222 | >10000 | >10000 | 311 ± 73 |
| 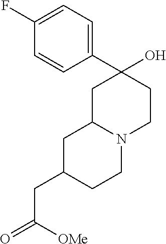 [SMILES: COC(=O)CC1CCN2CCC(O)(c3ccc(F)cc3)CC2C1] | 424 ± 203 | >10000 | >10000 | 240 ± 63 |
| 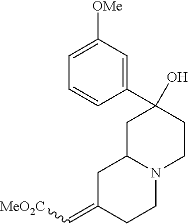 [SMILES: COC(=O)C=C1CCN2CCC(O)(c3cccc(OC)c3)CC2C1] | >10000 | >10000 | >10000 | 37 ± 3 |
| trans | | | | |
| 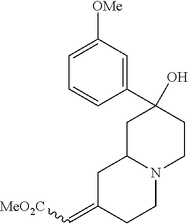 [SMILES: COC(=O)C=C1CCN2CCC(O)(c3cccc(OC)c3)CC2C1] | >10000 | >10000 | >10000 | 44 ± 5 |
| cis | | | | |
| Mitragynine | 709 ± 91.2 | 1530 ± 143 | 6800 ± 721 | 4090 ± 273 |
| Corynantheidine | 2520 ± 372 | 1034 ± 162 | 2406 ± 1200 | 68 ± 12 |
| DAMGO | 3.38 ± 0.436 | 2930 ± 879 | 655 ± 107 | NT |

NT = Not tested, MOR = mu opioid receptor, KOR = kappa opioid receptor, DOR = delta opioid
receptor.

After synthesizing COR analogs these were tested against MOR and α 2A receptors. Here better affinities towards MOR and α 2A receptors compared to mitragynine analog which is shown in table B2.

TABLE B2
Binding affinities of synthesised corynantheidine analogs at opioid and
α2-adrenergic receptor subtype

| | | MOR | Alpha2A | Alpha2C |
|---|---|---|---|---|
| Structure | Code | Ki ± SEM (nM) | Ki ± SEM (nM) | Ki ± SEM (nM) |
| 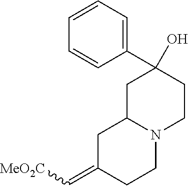 [SMILES: COC(=O)C=C1CCN2CCC(O)(c3ccccc3)CC2C1] | SG-01- 053 | >10,000 | 95 ± 35 | NT |
| 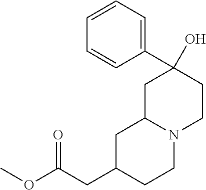 [SMILES: COC(=O)CC1CCN2CCC(O)(c3ccccc3)CC2C1] | SG-01- 049 | 9815 ± 2857 | 80 ± 19 | NT |
| 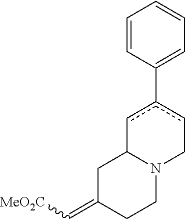 | SG-01- 0051A | 9016 ± 1508 | 6 ± 3 | NT |
| 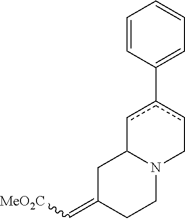 | SG-01- 0051B | 7989 ± 1683 | 8 ± 1 | NT |

| | | | |
|---|---|---|---|
| Mitragynine | 1530 ± 143 | 709 ± 91.2 | 4090 ± 372 |

The values represent the mean ± SEM from 3 independent experiments. (*). NT: Not tested

### EXPERIMENTAL

The solvents, reagents and starting materials were purchased from Fisher or Millipore-Sigma and were used without purification. Pre-coated silica gel 60 F254 aluminum backed plates from Millipore-Sigma were used for thin-layer chromatography (TLC). Compounds were purified by automated column chromatography were using a Teledyne ISCO apparatus (CombiFlash® R_{f}+) with RediSep® prepacked silica gel columns (4-120 g). 1H and 13C NMR spectra were obtained on a Bruker Avance Neo 600 MHz console. Spectra were acquired at 25° C. Chemical shifts values (6) were recorded in parts per million (ppm) and coupling constants (J) in Hertz (Hz). Signal multiplicities are abbreviated as follow: s (singlet), d (doublet), t (triplet), q (quartet), td (triple doublet), tt (triplet of triplets), m (multiplet).

1-benzyl-4-(2-methoxyphenyl)piperidin-4-ol (SG-01-001). A solution of 1-benzyl 4-piperidone (1.89 g) in dry tetrahydrofuran (5 ml) was added drop wise to a 1M solution of (2-methoxyphenyl) magnesium bromide (16 ml) in THF. The mixture was stirred for 3 h at room temperature. The reaction was quenched by the addition of saturated ammonium chloride solution and extracted into ethyl acetate (3×30 mL). The organic phases were combined and dried over anhydrous Na₂SO₄, filtered and concentrated under reduced pressure to furnish crude 1-benzyl-4-(2-methoxyphenyl)piperidin-4-ol. Purification of the title compound was achieved by flash chromatography on silica or, where indicated, by elution through a short length silica column using the eluent specified. 1-benzyl-4-(2-methoxyphenyl)piperidin-4-ol, Yield (2.4 g, 86%). ¹HNMR (600 MHz, CDCl₃): δ 2.02-2.05 (m, 2H), δ 2.08-2.13 (m, 2H), 2.57-2.61 (m, 2H), 2.74-2.76 (m, 2H), 3.59 (s, 2H), 3.90 (s, 3H), 4.02 (brs, 1H), 6.92-6.97 (m, 2H), 7.23-7.26 (m, 2H), 7.27-7.30 (m, 1H), 7.31-7.34 (m, 2H), 7.36-7.37 (m, 2H); ¹³C NMR (151 MHz, CDCl₃): δ (ppm)=157.2, 139.1, 135.5, 129.6, 128.5, 127.2, 125.9, 121.5, 111.7, 71.5, 63.7, 55.6, 49.6, 36.6. MS (ESI⁺): m/z=298.1 [M+H]⁺.

4-(2-methoxyphenyl)piperidin-4-ol (SG-01-002). To a solution of SG-01-001 (1.7 g) in ethanol (25 ml), 740 mg of Pd/C was added. H₂ gas was passed through balloon and stirred it for 16 hrs. After completion of the reaction, the reaction mixture was filtered through celite. The filtrate was concentrated in vacuum to afford 1 g of pure product SG-01-002. ¹HNMR (600 MHz, CDCl₃): δ 2.06-2.08 (m, 2H), δ 2.46-2.51 (m, 2H), 3.29-3.35 (m, 2H), 3.41-3.46 (m, 2H), 3.89 (s, 3H), 6.92-6.94 (m, 1H), 6.95-6.98 (m, 1H), 7.25-7.29 (m, 1H), 7.30-7.32 (m, 1H); ¹³C NMR (151 MHz, CDCl₃): δ (ppm)=157.0, 135.3, 128.1, 125.5, 125.5, 121.1, 111.3, 71.5, 55.2, 42.0, 36.9, MS (ESI⁺): m/z=208.3 [M+H]⁺.

1-(but-3-ynyl)-4-(2-methoxyphenyl)piperidin-4-ol (SG-01-003). To a mixture of compound SG-01-002 (1.0 g, 1 eq) and K₂CO₃ (1.3 g, 2 equiv.) in THF (20 mL), 1-Bromo-3-butyne (630 mg, 1 equiv.) was added dropwise. The mixture was stirred overnight under reflux, and then filtered and evaporated. The residue was purified by column chromatography DCM:MeOH 95:5. Yield (920 mg, 73%). ¹HNMR (600 MHz, CDCl₃): δ 2.02 (t, 1H, 2.7 Hz), δ 2.07-2.09 (m, 2H), 2.17 (td, 2H, J₁=4.2 Hz, J₂=12.9 Hz), 2.50 (td, 2H, J₁=2.7 Hz, J₂=7.6 Hz), 2.71-2.76 (m, 4H), 2.86 (d, 2H, J=10.6 Hz), 3.92 (s, 3H), 4.09 (brs, 1H), 6.94-6.99 (m, 2H, 7.25-7.30 (m, 2H); ¹³C NMR (151 MHz, CDCl₃): δ (ppm)=157.2, 134.7, 128.4, 125.6, 121.3, 111.4, 82.7, 70.7, 69.2, 57.0, 55.3, 48.9, 35.8, 16.6. MS (ESI⁺): m/z=260.2 [M+H]⁺.

8-hydroxy-8-(2-methoxyphenyl)hexahydro-1H-quinolizin-2(6H)-one (SG-01-004). To a stirred mixture of compound SG-01-003 (400 mg) and 4 A molecular sieves (3 g) in anhydrous DCM (10 ml) at 0 C under argon was added 3-chloroperbenzoic acid (mCPBA) (348 mg). After addition, the reaction mixture was stirred at 0 C for 1.5 h. After this, to the reaction mixture was added [Bis(trifluoromethanesulfonyl)imidate](triphenylphosphine)gold(I) catalyst (5 mol %) (120 mg) and stirred at 0 C for another 4.5 h. The reaction mixture was diluted with DCM (50 ml) and washed with 5% aqueous sodium carbonate. The organic layer was then dried over anhydrous sodium sulfate and concentrated under reduced pressure. The resulting residue was then purified by flash column chromatography on silica gel with MeOH:EtOAc 1:10 to afford 260 mg of compound SG-01-004 as an off white solid. Yield (260 mg, 61%). ¹HNMR (600 MHz, CDCl₃): δ 7.26-7.28 (m, 2H), δ 6.94-6.97 (m, 2H), 4.26 (s, 1H), 3.91 (s, 3H) 3.11-3.14 (m, 1H), 2.98-3.01 (m, 1H), 2.65-2.71 (m, 2H), 2.60-2.63 (m, 1H), 2.48-2.51 (m, 1H), 2.42-2.46 (m, 1H), 2.33-2.39 (m, 2H), 2.27-2.31 (m, 2H), 2.20-2.25 (m, 1H), 2.10-2.16 (m, 1H), 1.84-1.88 (m, 1H); ¹³C NMR (151 MHz, CDCl₃): δ (ppm)=208.6, 157.4, 131.8, 128.9, 127.8, 120.8, 111.7, 71.6, 59.6, 55.3, 54.7, 48.0, 44.5, 41.2, 37.2; MS (ESI⁺): m/z=276.2 [M+H]⁺.

Methyl 2-(8-hydroxy-8-(2-methoxyphenyl)dihydro-1H-quinolizin-2(6H,7H,8H,9H, 9aH)-ylidene)acetate (SG-01-005). To a suspension of sodium hydride (60 mg, 1.5 equiv.) in THF (10 ml) was added a solution of trimethyl phosphonoacetate (446 mg, 1.35 equiv.) in THF (5 mL) drop wise over 30 min. After complete addition the solution was stirred for 10 min then a solution of SG-01-004 (500 mg, 1 equiv.) in THF (5 mL) was added drop wise over 20 min. After 3 hrs of stirring at room temperature, the reaction mixture was quenched with few drops of H₂O, evaporated the solvent and extracted with ethyl acetate. The organic phase were washed with H₂O, brine, dried over Na₂SO₄, filtered, and purified through column chromatography (EtOAc MeOH) to give 480 mg of title compound SG-01-005 (yield 79%). While doing column chromatography, we found that there were two spots which were very close to each other. We managed to isolate both and labeled it as SG-01-005A an SG-01-005B.

SG-01-005A. ¹HNMR (600 MHz, CDCl₃): δ 7.28-7.29 (m, 1H), 7.24-7.27 (m, 1H), δ 6.93-6.96 (m, 2H), 5.61 (s, 1H), 3.90 (s, 3H), 3.75-3.78 (m, 1H), 3.66 (s, 3H), 2.97-3.00 (m, 1H), 2.86-2.89 (m, 1H), 2.56-2.59 (m, 1H), 2.48 (dt, 1H, J₁=2.7 Hz, J₂=13.6 Hz), 2.22-2.28 (m, 2H), 2.09-2.16 (m, 3H), 2.01-2.07 (m, 2H), 1.79-1.83 (m, 1H); ¹³C NMR (151 MHz, CDCl₃): δ (ppm)=167.0, 158.9, 157.4, 132.1, 128.7, 128.0, 120.7, 113.9, 111.6, 72.0, 60.7, 55.5, 55.3, 51.0, 44.4, 43.7, 37.2, 29.4; MS (ESI⁺): m/z=332.1 [M+H]⁺.

SG-01-005B. ¹HNMR (600 MHz, CDCl₃): δ 7.29 (dd, 1H, J₁=1.7 Hz, J₂=7.6 Hz), 7.23-7.26 (m, 1H), δ 6.92-6.95 (m, 2H), 5.62 (s, 1H), 3.90 (s, 3H), 3.73-3.78 (m, 1H), 3.67 (s, 3H), 2.97-3.00 (m, 1H), 2.87 (dt, 1H, J₁=3.8 Hz, J₂=11.7 Hz), 2.51-2.58 (m, 3H), 2.15-2.20 (m, 2H), 2.09-2.12 (m, 1H), 2.04-2.08 (m, 2H), 1.93-1.98 (m, 2H), 1.81-1.85 (m, 1H); ¹³C NMR (151 MHz, CDCl₃): δ (ppm)=166.9, 158.9, 157.2, 131.9, 128.4, 127.8, 120.5, 113.5, 111.4, 71.8, 59.7, 55.1, 55.3, 50.8, 44.6, 36.9, 36.4, 35.9; MS (ESI⁺): m/z=332.1 [M+H]⁺.

Methyl 2-(8-hydroxy-8-(2-methoxyphenyl)octahydro-1H-quinolizin-2-yl)acetate (SG-01-006). To a solution of SG-01-005 (480 mg) in methanol (20 ml), 154 mg of Pd/C was added. To this mixture H₂ gas was passed through balloon and stirred it for 1 hr. After completion of the reaction, the reaction mixture was filtered through celite. The filtrate was concentrated in vacuum to afford product SG-01-006. The crude was purified through column chromatography DCM:MeOH: 95: 0.5. Yield (450 mg, 93%). ¹HNMR (600 MHz, CDCl₃): δ 7.29-7.33 (m, 1H), 7.24-7.28 (m, 1H), δ 6.92-6.98 (m, 2H), 4.25 (brs, 1H), 3.89 (s, 3H), 3.65 (s, 3H), 2.65-2.87 (m, 2H), 2.52-2.57 (m, 1H), 2.32-2.46 (m, 2H), 2.17-2.26 (m, 2H), 1.97-2.13 (m, 3H), 1.90-1.96 (m, 1H), 1.76-1.85 (m, 1H), 1.72 (t, 1H, J=12.5 Hz), 1.64-1.69 (m, 1H), 1.32-1.61 (m, 2H); ¹³C NMR (151 MHz, CDCl₃): δ (ppm)=173.3, (173.0), 157.2, 132.2, (132.1), 128.4, 127.9, (127.8), 120.5, (120.4), 111.4, (111.3), 72.1, (72.0), 59.1, 55.0, 54.0, 51.4, (51.4), 44.4, 40.9, 39.3, 37.1, 32.7, 27.9; MS (ESI⁺): m/z=334.4 [M+H]⁺.

Methyl 2-(8-(2-methoxyphenyl)-2,3,4,6,9/7,9a-hexahydro-1H-quinolizin-2-yl)acetate and SG-01-007. For alcohol dehydration reaction, compound SG-01-006 (450 mg) was treated with (10 ml) trifluoroacetic acid: dichloromethane (1:1) and the mixture was refluxed for 3 h. After completion of the reaction, the reaction was neutralized with saturated sodium carbonate solution and extracted with dichloromethane (3×30 ml). The organic phase were dried over Na₂SO₄, filtered, and purified through column chromatography (EtOAc MeOH) to give 307 mg of title compound SG-01-007. While doing column chromatography, we found that there were two spots which were very close to each other. We managed to isolate both and labeled it as SG-01-007A an SG-01-007B.

SG-01-007A. ¹HNMR (600 MHz, CDCl₃): δ 7.20-7.23 (m, 1H), 7.15 (dd, 1H, J₁=7.4 Hz, J₂=1.8 Hz), 6.90 (dt, 1H, J₁=7.4 Hz, J₂=1.1 Hz), 6.85-6.86 (m, 1H), 5.53 (brs, 1H), 3.79 (s, 3H), 3.68 (s, 3H), 2.89-2.95 (m, 2H), 2.77-2.83 (m, 1H), 2.66-2.69 (m, 1H), 2.49-2.53 (m, 1H), 2.38-2.42 (m, 1H), 2.28 (d, 2H, 7.2 Hz), 2.24-2.27 (m, 1H), 1.94-2.02 (m, 1H), 1.75-1.78 (m, 3H), 1.45-1.52 (m, 1H); ¹³C NMR (151 MHz, CDCl₃): δ (ppm)=173.1, 156.8, 135.3, 131.5, 129.4, 128.3, 128.1, 120.5, 110.7, 61.1, 55.3, 55.2, 52.2, 51.4, 41.1, 38.2, 33.6, 32.0, 29.4; MS (ESI⁺): m/z=316.4 [M+H]⁺.

SG-01-007B. ¹HNMR (600 MHz, CDCl₃): δ 7.20-7.23 (m, 1H), 7.13-7.15 (m, 1H), 6.88-6.90 (m, 1H), 6.84-6.85 (m, 1H), 5.72-5.74 (m, 1H), 3.79 (s, 3H), 3.67 (s, 3H), 3.42-3.45 (m, 1H), 3.03 (dt, 1H J₁=11.4 Hz, J₂=3.4 Hz), 2.88-2.92 (m, 1H), 2.44-2.52 (m, 1H), 2.28-2.32 (m, 2H), 2.25-2.27 (m, 2H), 2.14 (td, 1H, J₁=12.5 Hz, J₂=2.7 Hz), 1.89-1.96 (m, 1H), 1.82-1.85 (m, 1H), 1.74-1.78 (m, 1H), 1.39-1.46 (m, 1H), 1.03-1.09 (m, 1H); ¹³C NMR (151 MHz, CDCl₃): δ (ppm)=173.5, 157.2, 135.3, 132.0, 130.0, 128.5, 123.3, 120.9, 111.0, 57.2, 55.7, 55.6, 55.2, 51.8, 41.5, 40.0, 37.0, 33.3, 32.4; MS (ESI⁺): m/z=316.4 [M+H]

Methyl 2-(8-(2-methoxyphenyl)octahydro-1H-quinolizin-2-yl)acetate (SG-01-008). To a solution of SG-01-007 (300 mg) in methanol (15 ml), 95 mg of Pd/C was added. To this mixture H₂ gas was passed through balloon and stirred it for 5 hr. After completion of the reaction, the reaction mixture was filtered through celite. The filtrate was concentrated in vacuum to afford 285 mg of SG-01-008, ¹HNMR (600 MHz, CDCl₃): δ 7.17-7.24 (m, 2H), 6.91-6.95 (m, 1H), δ 6.83-6.87 (m, 1H), 3.81 (s, 3H), 3.67 (s, 3H), 3.39-3.43 (m, 1H), 3.24-3.34 (m, 2H), 3.07-3.19 (m, 1H), 2.85-2.94 (m, 1H), 2.38-2.59 (m, 3H), 2.27-2.33 (m, 1H), 2.13-2.26 (m, 2H), 1.96-2.13 (m, 1H), 1.74-1.93 (m, 3H), 1.57-1.70 (m, 2H); ¹³C NMR (151 MHz, CDCl₃): δ (ppm)=173.6, (173.2), 157.4, 133.6, (128.2), 128.1, 126.9, 120.1, (120.0), 110.4, 57.6, 55.6, 55.3, 52.5, (51.6), 41.4, 36.6, (36.5), 33.5, 29.8, 28.7; MS (ESI⁺): m/z=316.4 [M+H]⁺.

Methyl 3-hydroxy-2-(8-(2-methoxyphenyl)octahydro-1H-quinolizin-2-yl)acrylate (SG-01-009). To a solution of 2 M solution of LDA in THF (2.3 ml, 5 equiv.) at −78 C, a solution of ester SG-01-008 (285 mg) in anhydrous THF (3 ml) was added dropwise over 15 min. After stirring the orange solution for 1.5 h at −78° C., anhydrous methyl formate (6 ml, 110 equiv.) was added, and the mixture was warmed to 0° C. and stirred for 1 h. The reaction was then quenched with water (50 ml) and extracted with CH₂Cl₂ (4×25 mL). The extracted fractions were combined, dried over Na₂SO₄, and concentrated to provide a viscous, yellow-orange oil (-350 mg). We tried to purify the compound but failed to get out of column. We confirmed the compound through mass spectroscopy and tried to record crudes NMR and the spectrum was extremely complex due to mixture of tautomers and E/Z enol. So we decided to use this crude for next step without further purification.

Methyl 3-methoxy-2-(8-(2-methoxyphenyl)octahydro-1H-quinolizin-2-yl)acrylate (SG-01-040). To a flask cooled to 0° C. was added SG-01-009 (350 mg) and dissolved in minimum amount of DMF (3 mL). Then, K₂CO₃ (207 mg, 1.5 equiv.) and dimethyl sulfate (95 μl, 1.0 equiv.) were added and the reaction was stirred at 0° C. for 1 h. The crude was extracted with diethyl ether, dried over Na₂SO₄, and evaporated to give crude SG-01-040 as a yellow oil (360 mg). The compound was then purified with column chromatography DCM:MeOH:95:5. ¹HNMR (600 MHz, CDCl₃): δ 7.31 (s, 1H), 7.23-7.24 (m, 1H), 7.16-7.19 (m, 1H), 6.92 (t, 1H, J 7.4 Hz), δ 6.84 (d, 1H, J=8.2 Hz), 3.82 (s, 3H), 3.81 (s, 3H), 3.70 (s, 3H), 3.57-3.66 (m, 1H), 3.21-3.29 (m, 2H), 3.12-3.17 (m, 1H), 2.77-2.82 (m, 1H), 2.53-2.55 (m, 1H), 2.36-2.49 (m, 3H), 2.17-2.27 (m, 2H), 1.79-1.87 (m, 3H), 1.55-1.62 (m, 2H); ¹³C NMR (151 MHz, CDCl₃): δ (ppm)=168.6, 160.1, 156.5, 132.1, 128.5, 127.2, 126.7, 120.7, 110.2, 63.9, 61.6, 56.0, 55.2, 51.3, 34.1, 33.2, 31.6, 29.6, 29.1, 22.6; MS (ESI⁺): m/z=360.2 [M+H]⁺.

### Radioligand Binding Assays

The kratom alkaloids were screened at Eurofins Cerep (Celle l'Evescault, France) for in vitro binding affinity at alpha adrenergic and opioid receptors (Table 2A and Table 2B). Briefly, each cell membrane homogenate was incubated with a radioligand in the absence or presence of the kratom alkaloids in a buffer. Nonspecific binding was determined in the presence of a specific agonist or antagonist at the target receptor. Following incubation, the samples were filtered rapidly under vacuum through glass fiber filters presoaked in a buffer and rinsed several times with an ice-cold buffer using a 48- or 96-sample cell harvester. The filters were counted for radioactivity in a scintillation counter using a scintillation cocktail. The IC₅₀ values (concentration causing a half-maximal inhibition of control specific binding) from the displacement data were computed using a nonlinear, least-squares regression analysis (Prism; GraphPad Software, Inc., San Diego, CA) as well as the concentration of radioligand used in the assay (Table 1), and the Eurofins Cerep's historical value for the K_{D} values of the radioligand. The IC₅₀ values converted to Kᵢ values using the Cheng-Prusoff equation.

### Compounds of Formula (VI), (VII), (VIII), and (IX)

### Synthesis

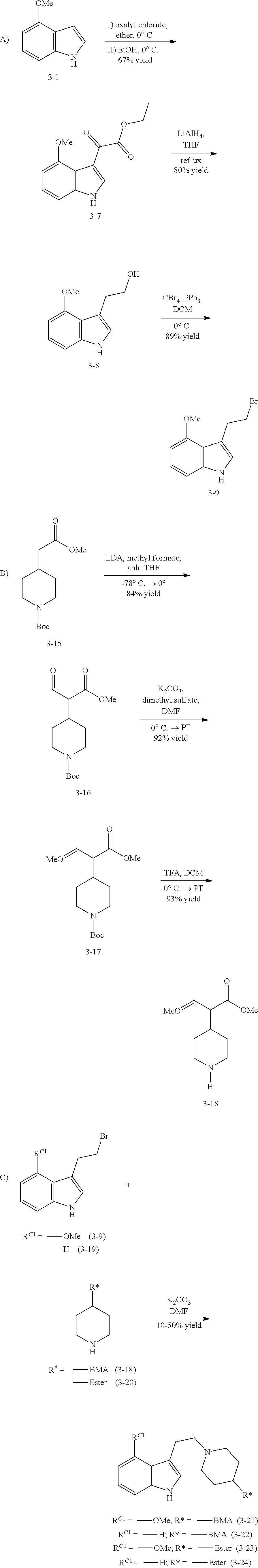

Scheme 3-1. Overview of Analog Synthesis. A) Synthesis of Indole Fragment. B) Synthesis of Piperidine Fragment. C) Synthesis of Parent Analogs (3-21 to 3-24).

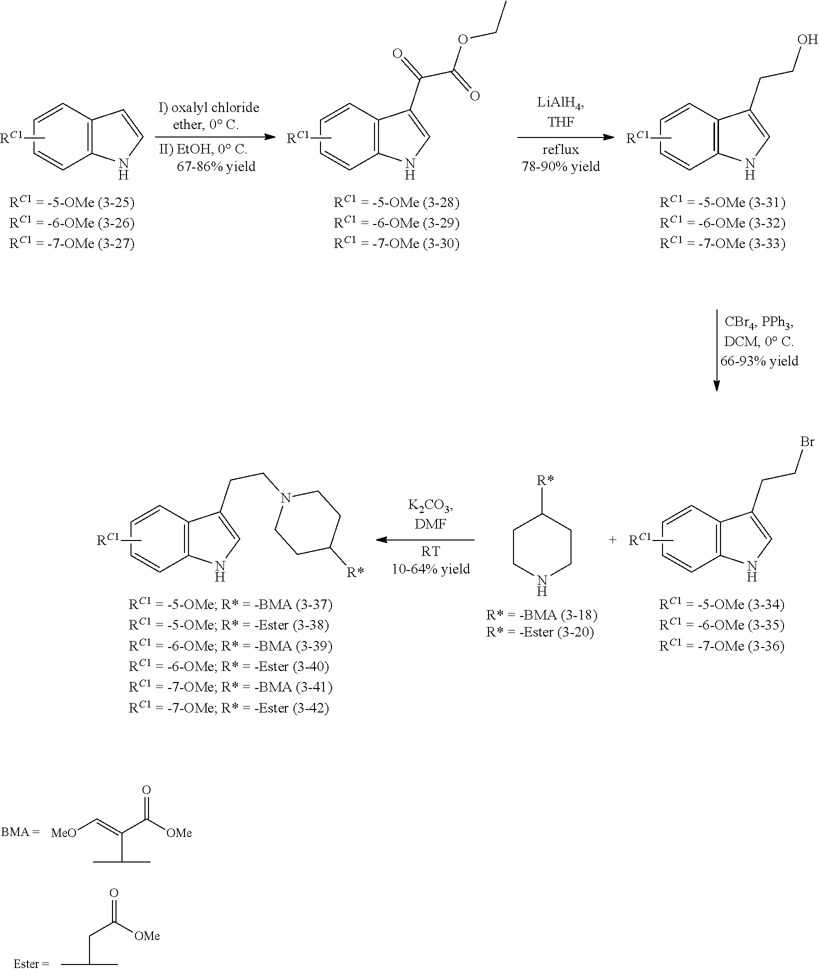

### Scheme 3-2. Synthesis of Indole Substituted Analogs.

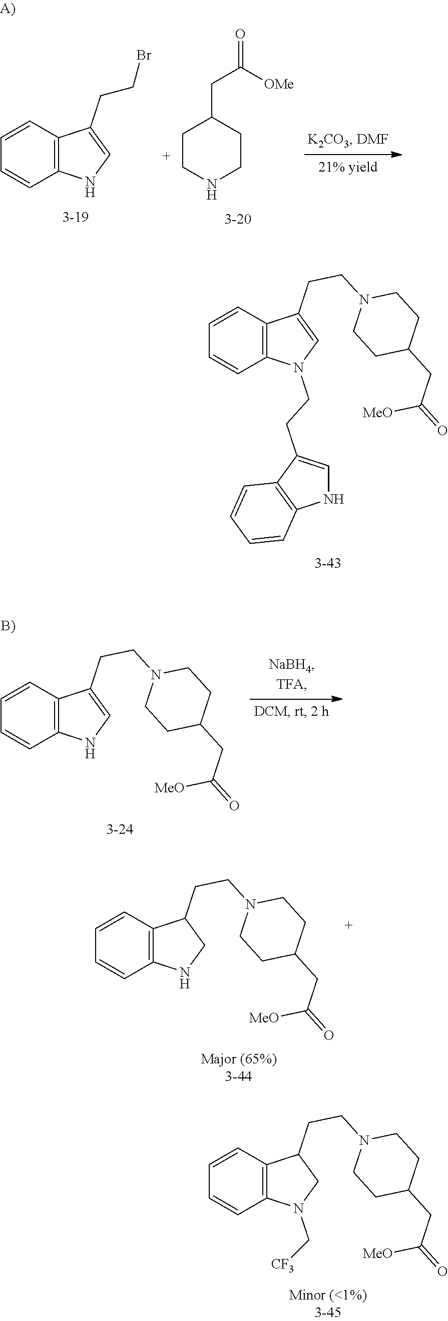

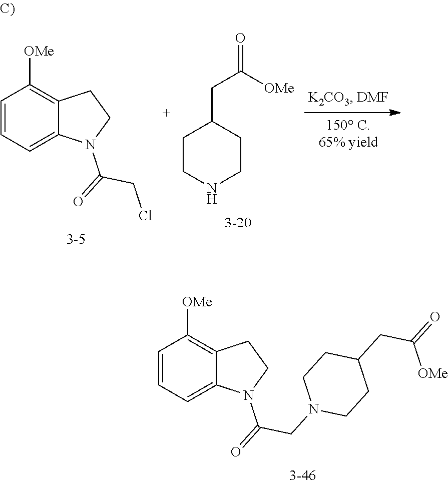

Scheme 3-3. Synthesis of Indoline and N-Substituted Analogs. A) Synthesis of Dimerized Analog 3-43. B) Synthesis of Unsubstituted Indoline and N-Substituted Indoline Analogs 3-44 and 3-45. C) Synthesis of Methoxy Substituted Indoline Analog 3-46.

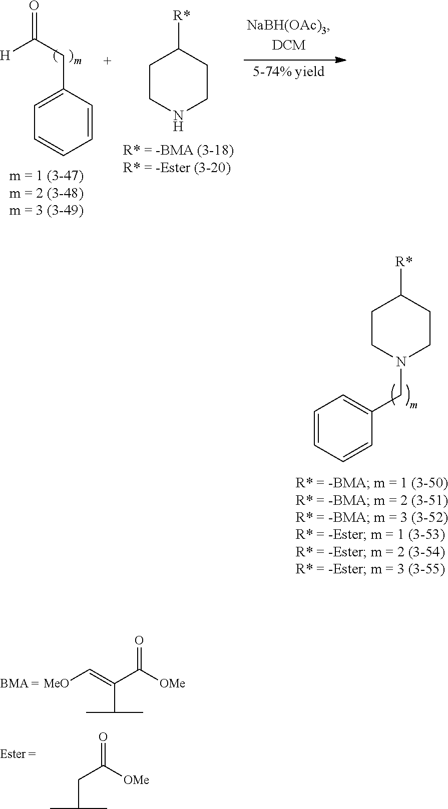

### Scheme 3-4. Synthesis of Phenyl Analogs 3-50 to 3-55.

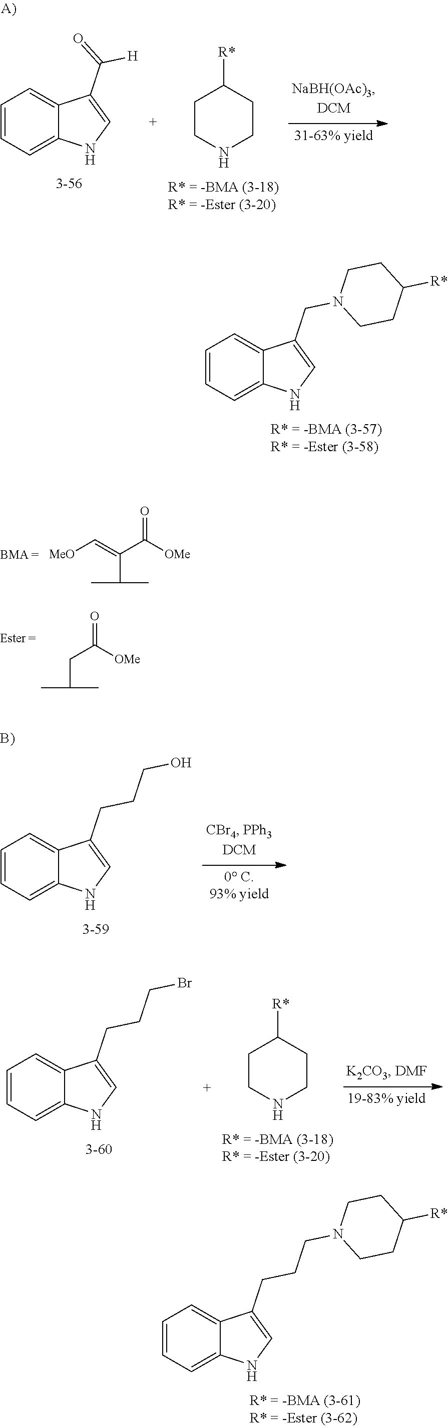

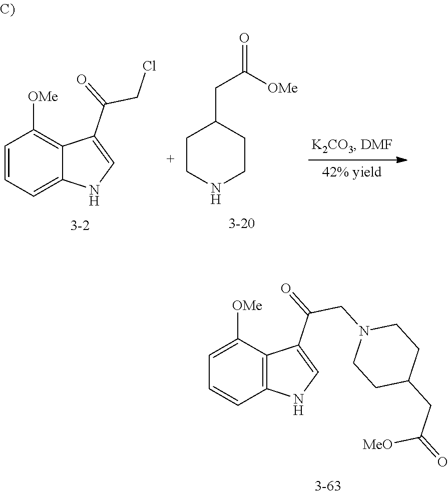

Scheme 3-5. Synthesis of Modified Ethyl Linker Analogs. A) Synthesis of Analogs with One Carbon Spacer (3-57 to 3-58). B) Synthesis Scheme for Analogs Bearing Three Carbon Linker (3-61 to 3-62). C) Synthesis of Analog with a Carbonyl Introduced on Ethyl Linker (3-63).

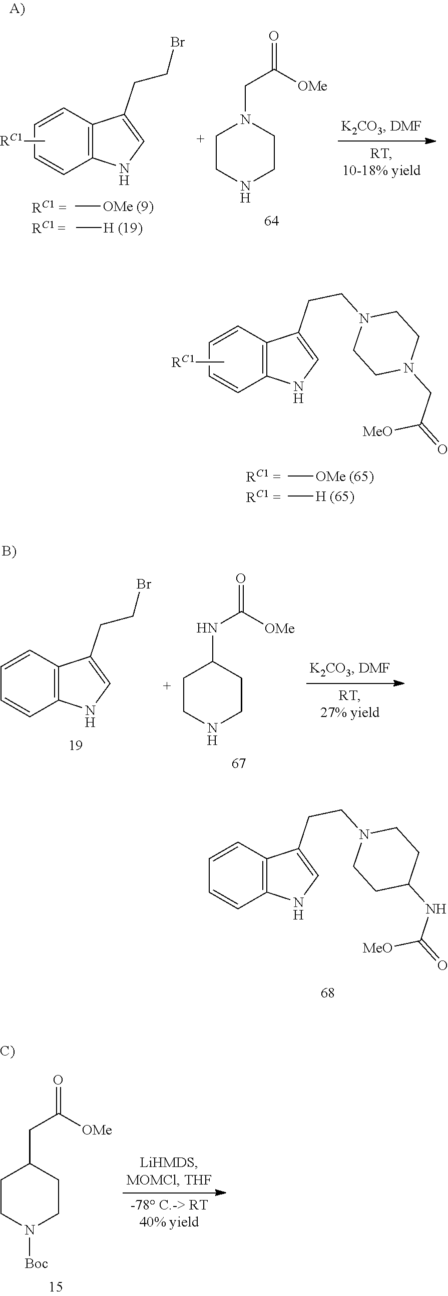

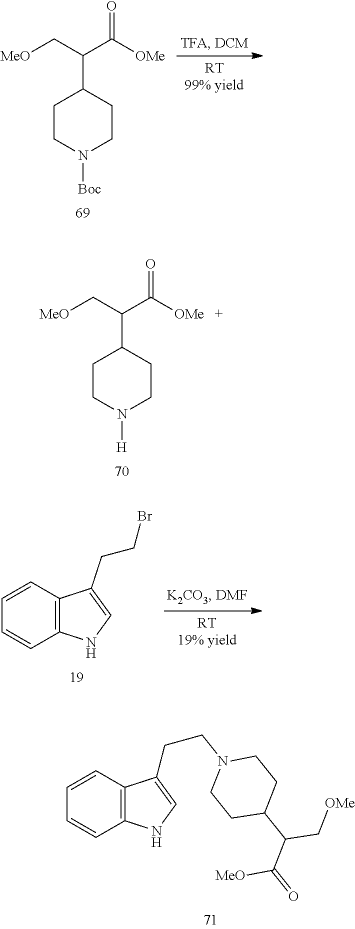

Scheme 3-6. Synthesis of Analogs with Modifications on the Piperidine and BMA Portion. A) Synthesis of Analogs (3-65 and 3-66) with a Piperazine Ring in Place of the Piperidine Ring. B) Synthesis of Analog (3-68) with the Ester Replaced by a Carbamate Group. C) Synthesis Scheme for Analog (3-71) with the Enol Ether on the BMA Moiety Reduced.

### Data

TABLE 3-1
Binding affinities of analogs at opioid and a2-adrenergic receptor subtypes

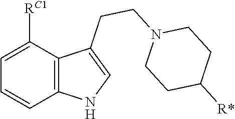

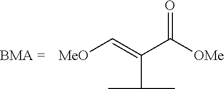

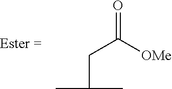

| Comp | | | Ki (nM ± SEM) | | | | | |
|---|---|---|---|---|---|---|---|---|
| # | R^{C1} | R* | MOR | KOR | DOR | a_{2A}-AR | a_{2C}-AR | SR |
| 1-MG | — | — | 709 ± 91.2 | 1530 ± 485 | 6800 ± 721 | 4090 ± 273 | 4040 ± 417 | 0.17 |
| 3-21 | OMe | BMA | 1480 ± 236 | 1450 ± 312 | >10,000 | 6680 ± 792 | NT | 0.22 |
| 3-22 | OMe | Ester | >10,000 | >10,000 | >10,000 | 229 ± 24.9* | 69.3 ± 4.2 | — |
| 1-COR | — | — | 342 ± 52.5 | 4060 ± 969 | 2790 ± 171 | 351 ± 58.7 | 135 ± 12.5 | 0.97 |
| 3-23 | H | BMA | 451 ± 108* | 3670 ± 1260 | >10,000 | 741 ± 19.3* | 830 ± 268 | 0.61 |
| 3-24 | H | Ester | 2230 ± 42.6 | >10,000 | >10,000 | 1410 ± 122 | NT | 1.58 |

The values represent the mean ± SEM from 3 independent experiments. (*): Submitted for functiona
l assessment. NT: Not tested due to the affinity at α_{2A}-AR being >1 μM. SR: Selectivity Ratio (Ki_{MOR}/
Ki_{α2A}). 1-MG = mitragynine = 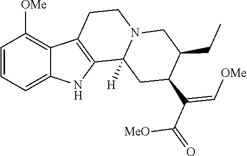 [SMILES: CC[C@@H]1CN2CCc3c([nH]c4cccc(OC)c34)[C@@H]2C[C@@H]1/C(=C\OC)C(=O)OC]
1-COR =

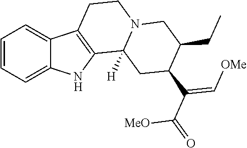 [SMILES: CC[C@@H]1CN2CCc3c([nH]c4ccccc34)[C@@H]2C[C@@H]1/C(=C\OC)C(=O)OC]

TABLE 3-2
Binding affinities of methoxy substituted analogs at opioid and a2-
adrenergic receptor subtypes

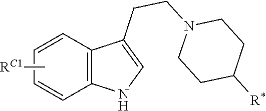

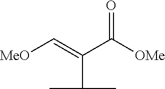

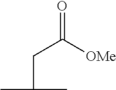

| Comp | | | Ki (nM ± SEM) | | | | | |
|---|---|---|---|---|---|---|---|---|
| # | R^{C1} | R* | MOR | KOR | DOR | a_{2A}-AR | a_{2C}-AR | SR |
| 3-23 | H | BMA | 451 ± 108* | 3670 ± 1260 | >10,000 | 741 ± 19.3* | 830 ± 268 | 0.61 |
| 3-21 | 4-OMe | BMA | 1480 ± 236 | 1450 ± 312 | >10,000 | 6680 ± 792 | NT | 0.22 |
| 3-37 | 5-OMe | BMA | 733 ± 144* | 1300 ± 129 | >10,000 | 3370 ± 586 | NT | 0.22 |
| 3-39 | 6-OMe | BMA | >10,000 | 9450 ± 2970 | >10,000 | >10,000 | NT | — |
| 3-41 | 7-OMe | BMA | 5130 ± 8.35 | >10,000 | >10,000 | 4900 ± 829 | NT | 1.04 |
| 3-24 | H | Ester | 2230 ± 42.6 | >10,000 | >10,000 | 1410 ± 122 | >10,000 | 1.58 |
| 3-22 | 4-OMe | Ester | >10,000 | >10,000 | >10,000 | 229 ± 24.9* | 69.3 ± 4.16 | — |
| 3-38 | 5-OMe | Ester | >10,000 | 8070 ± 810 | >10,000 | 1590 ± 225 | NT | — |
| 3-40 | 6-OMe | Ester | >10,000 | >10,000 | >10,000 | 2840 ± 599 | NT | — |
| 3-42 | 7-OMe | Ester | >10,000 | >10,000 | >10,000 | 1300 ± 129 | 830 ± 268 | — |

The values represent the mean ± SEM from 3 independent experiments. (*): Submitted for functional
assessment. S: Submitted for testing. NT: Not tested due to the affinity at a_{2A}-AR being >1 mM.
SR: Selectivity Ratio (Ki_{MOR}/Ki_{▭2A}).

TABLE 3-3
Binding affinities of indole-substituted and indoline analogs at opioid and a2-AR subtypes.

| Comp | | Ki (nM ± SEM) | | | | | |
|---|---|---|---|---|---|---|---|
| # | Structure | MOR | KOR | DOR | a_{2A}-AR | a_{2C}-AR | SR |
| 3-24 | 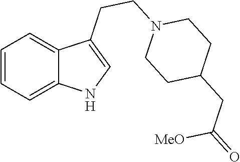 [SMILES: COC(=O)CC1CCN(CCc2c[nH]c3ccccc23)CC1] | 2230 ± 42.6 | >10,000 | >10,000 | 1410 ± 122 | >10,000 | 1.58 |
| 3-43 | 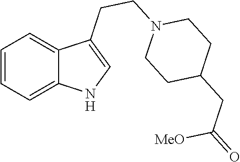 [SMILES: COC(=O)CC1CCN(CCc2c[nH]c3ccccc23)CC1] | 8910 ± 1803 | >10,000 | >10,000 | 1840 ± 201 | NT | 4.84 |
| 3-44 | 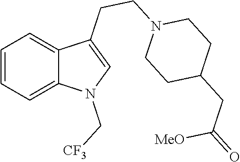 [SMILES: COC(=O)CC1CCN(CCc2cn(CC(F)(F)F)c3ccccc23)CC1] | 1480 ± 406 | >10,000 | >10,000 | 1940 ± 410 | NT | 0.76 |
| 3-45 | 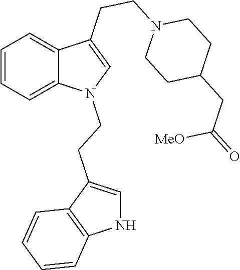 [SMILES: COC(=O)CC1CCN(CCc2cn(CCc3c[nH]c4ccccc34)c3ccccc23)CC1] | 9280 ± 1730 | >10,000 | >10,000 | 7030 ± 2020 | NT | 1.32 |
| 3-22 | 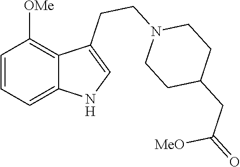 [SMILES: COC(=O)CC1CCN(CCc2c[nH]c3cccc(OC)c23)CC1] | >10,000 | >10,000 | >10,000 | 229 ± 24.9* | 69.3 ± 4.16 | — |
| 3-46 | 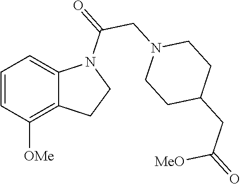 [SMILES: COC(=O)CC1CCN(CC(=O)N2CCc3c(OC)cccc32)CC1] | >10,000 | >10,000 | >10,000 | 6370 ± 1160 | NT | — |
| 3-42 | 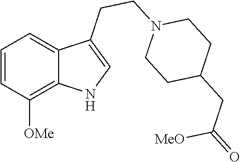 [SMILES: COC(=O)CC1CCN(CCc2c[nH]c3c(OC)cccc23)CC1] | >10,000 | >10,000 | >10,000 | 1300 ± 129 | NT | — |

The values represent the mean±SEM from 3 independent experiments. (*): Submitted for functional assessment. S: Submitted for testing. NT: Not tested due to the affinity at α_{2A}-AR being >1 μM. SR: Selectivity Ratio (Ki_{MOR}/Kiα_{2A}).

TABLE 3-4
Binding affinities of phenylalkyl-substituted analogs at opioid and a2-
adrenergic receptor subtypes.

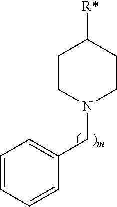

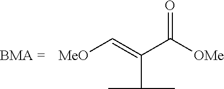

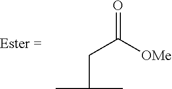

| | | | Ki (nM ± SEM) | | | | | |
|---|---|---|---|---|---|---|---|---|
| Comp # | m | R* | MOR | KOR | DOR | a_{2A}-AR | a_{2C}-AR | SR |
| 3-23 | — | — | 451 ± 108* | 3670 ± 1260 | >10,000 | 741 ± 19.3* | 830 ± 268 | 0.61 |
| 3-50 | 1 | BMA | >10,000 | 5700 ± 558 | >10,000 | >10,000 | NT | — |
| 3-51 | 2 | BMA | 227 ± 57* | 5140 ± 3120 | >10,000 | 928 ± 155* | S | 0.24 |
| 3-52 | 3 | BMA | 409 ± 20* | 1340 ± 470 | >10,000 | 8060 ± 1870 | NT | 0.05 |
| 3-24 | — | — | 2230 ± 42.6 | >10,000 | >10,000 | 1410 ± 122 | >10,000 | 1.58 |
| 3-53 | 1 | Ester | >10,000 | 7250 ± 4270 | >10,000 | 2030 ± 528 | NT | — |
| 3-54 | 2 | Ester | 819 ± 78* | >10,000 | >10,000 | 2950 ± 591 | NT | 0.28 |
| 3-55 | 3 | Ester | >10,000 | >10,000 | >10,000 | 668 ± 81.5* | S | — |

The values represent the mean ± SEM from 3 independent experiments. (*): Submitted for functional
assessment. S: Submitted for testing. NT: Not tested due to the affinity at α_{2A}-AR being >1 μM.
S: Submitted for in vitro testing. SR: Selectivity Ratio (Ki_{MOR}/Ki_{α2A}).

TABLE 3-5
Binding affinities of the linker modified analogs at opioid and a2-
adrenergic receptor subtypes

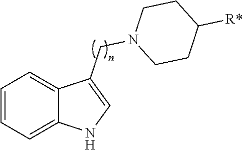

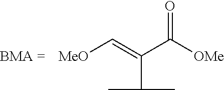

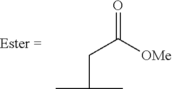

| Comp | | | Ki (nM ± SEM) | | | | | |
|---|---|---|---|---|---|---|---|---|
| # | n | R* | MOR | KOR | DOR | a_{2A}-ar | a_{2C}-AR | SR |
| 3-57 | 1 | BMA | >10,000 | >10,000 | >10,000 | 867 ± 370* | S | — |
| 3-23 | 2 | BMA | 451 ± 108* | 3670 ± 1260 | >10,000 | 741 ± 19.3* | 830 ± 268 | 0.61 |
| 3-61 | 3 | BMA | 6760 ± 3880 | >10,000 | >10,000 | 877 ± 327* | S | 7.71 |
| 3-58 | 1 | Ester | 2700 ± 580 | 791 ± 216 | >10,000 | 3460 ± 428 | NT | 0.78 |
| 3-24 | 2 | Ester | 2230 ± 42.6 | >10,000 | >10,000 | 1410 ± 122 | >10,000 | 1.58 |
| 3-62 | 3 | Ester | 2180 ± 364 | 2520 ± 795 | >10,000 | 2810 ± 544 | NT | 0.78 |

| | | | | | | | |
|---|---|---|---|---|---|---|---|
| 3-22 | 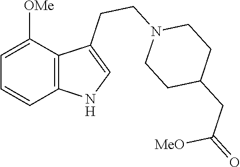 [SMILES: COC(=O)CC1CCN(CCc2c[nH]c3cccc(OC)c23)CC1] | >10,000 | >10,000 | >10,000 | 229 ± 24.9* | 69.3 ± 4.16 | — |
| 3-63 | 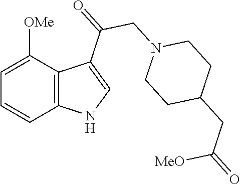 [SMILES: COC(=O)CC1CCN(CC(=O)c2c[nH]c3cccc(OC)c23)CC1] | >10,000 | >10,000 | >10,000 | 6370 ± 1160 | NT | — |

The values represent the mean ± SEM from 3 independent experiments. (*): Submitted for functional assessment. S: Submitted for testing.
NT: Not tested due to the affinity at α_{2A}-AR being >1 μM. S: Submitted for in vitro testing. SR: Selectivity Ratio (Ki_{MOR}/Ki_{α2A}).

TABLE 3-6
Binding affinities of piperidine, ester, and BMA modified analogs at opioid and a2-adrenergic receptor substypes.

| Comp | | Ki (nM ± SEM) | | | | | |
|---|---|---|---|---|---|---|---|
| # | Structure | MOR | KOR | DOR | a_{2A}-AR | a_{2C}-AR | SR |
| 22 | 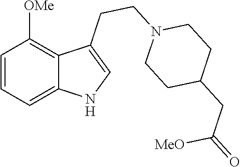 [SMILES: COC(=O)CC1CCN(CCc2c[nH]c3cccc(OC)c23)CC1] | >10,000 | >10,000 | >10,000 | 229 ± 24.9 | 69.3 ± 4.16 | — |
| 65 | 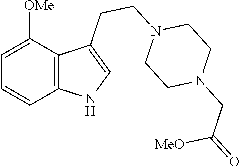 [SMILES: COC(=O)CN1CCN(CCc2c[nH]c3cccc(OC)c23)CC1] | >10,000 | >10,000 | >10,000 | 265 ± 64.3 | S | — |
| 24 | 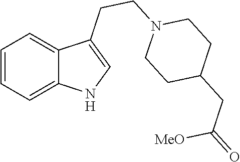 [SMILES: COC(=O)CC1CCN(CCc2c[nH]c3ccccc23)CC1] | 2230 ± 42.6 | >10,000 | >10,000 | 1410 ± 122 | >10,000 | 1.58 |
| 66 | 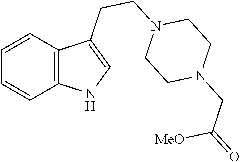 [SMILES: COC(=O)CN1CCN(CCc2c[nH]c3ccccc23)CC1] | 2030 ± 694 | >10,000 | >10,000 | 9760 ± 4830 | NT | 0.21 |
| 68 | 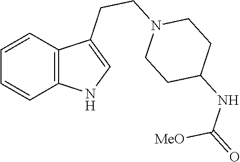 [SMILES: COC(=O)NC1CCN(CCc2c[nH]c3ccccc23)CC1] | 1890 ± 454 | >10,000 | >10,000 | 5090 ± 730 | NT | 0.37 |
| 23 | 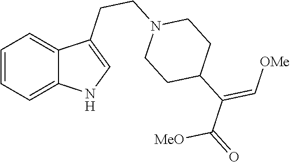 [SMILES: CO/C=C(/C(=O)OC)C1CCN(CCc2c[nH]c3ccccc23)CC1] | 451 ± 108 | 3670 ± 1260 | >10,000 | 741 ± 19.3 | 135 ± 12.5 | 0.61 |
| 71 | 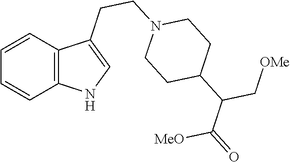 [SMILES: COCC(C(=O)OC)C1CCN(CCc2c[nH]c3ccccc23)CC1] | 5580 ± 1700 | >10,000 | >10,000 | 2760 ± 281 | NT | 2.02 |

The values represent the mean ± SEM from 3 independent experiments. (*): Submitted for functional assessment. S: Submitted for testing.
NT: Not tested due to the affinity at α_{2A}-AR being >1 μM. S: Submitted for in vitro testing. SR: Selectivity Ratio (Ki_{MOR}/Ki_{α2A}).

TABLE 3-7
In vitro binding and functional assessment
of 21 and 24 at human MOR.

| | MOR | MOR | MOR |
|---|---|---|---|
| | Ki (nM ± | EC₅₀ ± | Eₘₐₓ ± |
| Comp # | SEM) | SEM (nM) | SEM (%) |
| DAMGO | 3.17 ± 0.4 | 38.1 ± 6.5 | 106 ± 2 |
| MG | 709 ± 91 | ND | 3.97 ± 9.5 |
| COR | 450 ± 100 | ND | 8.32 ± 3.4 |
| 21 | 1480 ± 236 | ND | −1.03 ± 1.1 |
| 24 | 2230 ± 43 | 1220 ± 3670 | 23.8 ± 6.7 |
| DAMGO + Ntx | — | 2820 ± 917 | 91.7 ± 5.4 |
| DAMGO + MG | — | 1160 ± 402 | 92.7 ± 8.4 |
| DAMGO + COR | — | 152 ± 48.7 | 102 ± 7.6 |
| DAMGO + 21 | — | 717 ± 171 | 110 ± 1.4 |

Agonist activity at MOR indicated by EC50 values; maximal efficacy (Emax) relative to DAMGO (100%). ND: Ki could not be determined because compound produced less than 50% displacement of bound radioligand at 10 μM.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I′): 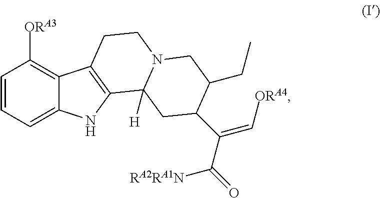
or pharmaceutically acceptable salt thereof, wherein:
R^{A1} is hydrogen, substituted or unsubstituted C₁₋₁₂ alkyl, −CD₃, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, —CD₃, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, —CD₃, or an oxygen protecting group; and
R^{A4} is hydrogen, substituted or unsubstituted C₁₋₁₂ alkyl, or an oxygen protecting group;
provided the compound is not of the formula:

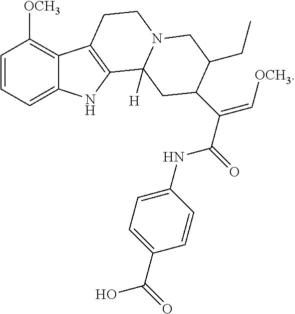 [SMILES: CCC1CN2CCc3c([nH]c4cccc(OC)c34)C2CC1/C(=C\OC)C(=O)Nc1ccc(C(=O)O)cc1]

2. The compound of claim 1, wherein the compound is of formula: 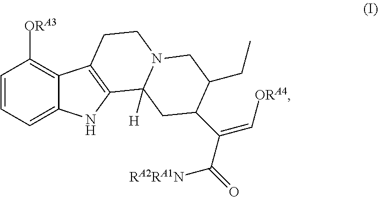
or pharmaceutically acceptable salt thereof.

3. The compound of claim 1, or pharmaceutically acceptable salt thereof, wherein the compound of Formula (I′) is of Formula (I): 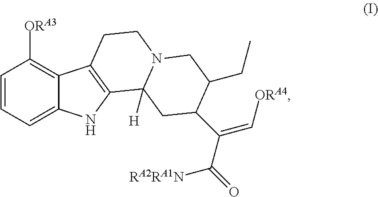
or pharmaceutically acceptable salt thereof, wherein:
R^{A1} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, substituted or unsubstituted 3- to 13-membered carbocyclyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, or an oxygen protecting group; and
R^{A4} is hydrogen, substituted or unsubstituted C₁₋₁₂ alkyl, or an oxygen protecting group;
provided the compound is not of the formula:

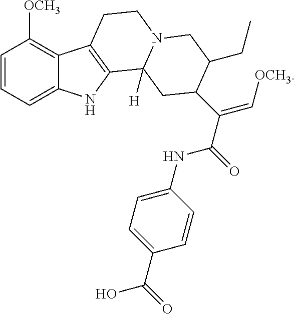 [SMILES: CCC1CN2CCc3c([nH]c4cccc(OC)c34)C2CC1/C(=C\OC)C(=O)Nc1ccc(C(=O)O)cc1]

4. The compound of claim 3, or pharmaceutically acceptable salt thereof, wherein the compound of Formula (I) is of Formula (I-A): 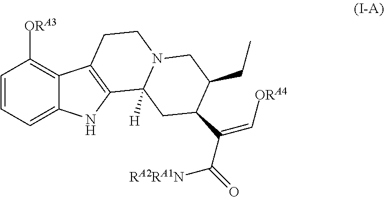
or pharmaceutically acceptable salt thereof.

5. The compound of claim 3, or pharmaceutically acceptable salt thereof, wherein the compound of Formula (I) is of Formula (I-B): 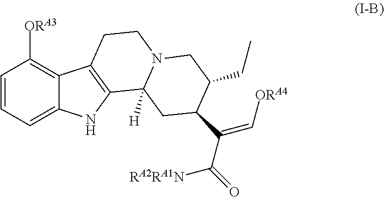
or pharmaceutically acceptable salt thereof.

6. The compound of any one of claims 3-5, or pharmaceutically acceptable salt thereof, wherein R^{A3} is C₁₋₆ alkyl.

7. The compound of any one of claims 3-5, or pharmaceutically acceptable salt thereof, wherein R^{A3} is methyl.

8. The compound of any one of claims 3-7, or pharmaceutically acceptable salt thereof, wherein R^{A4} is C₁₋₆ alkyl.

9. The compound of any one of claims 3-8, or pharmaceutically acceptable salt thereof, wherein R^{A4} is methyl.

10. The compound of any one of claims 3-9, or pharmaceutically acceptable salt thereof, wherein R^{A1} is hydrogen or unsubstituted C₁₋₆ alkyl.

11. The compound of any one of claims 3-10, or pharmaceutically acceptable salt thereof, wherein R^{A1} is hydrogen, methyl, ethyl, n-propyl, or isopropyl.

12. The compound of any one of claims 3-9, or pharmaceutically acceptable salt thereof, wherein R^{A1} is C₁₋₆ alkyl substituted with one or more groups selected from —COOMe, —COOEt, fluoro, chloro, bromo, iodo, —NH₂, —NMe₂, —NEt₂, —OMe, —OEt, and phenyl.

13. The compound of any one of claims 3-9, or pharmaceutically acceptable salt thereof, wherein R^{A1} is cyclopentyl or cyclohexyl.

14. The compound of any one of claims 3-9, or pharmaceutically acceptable salt thereof, wherein R^{A1} is phenyl substituted with one or more groups selected from —COOMe, —COOEt, fluoro, chloro, bromo, iodo, —NH₂, —NMe₂, —NEt₂, —OMe, —OEt, and phenyl.

15. The compound of any one of claims 3-14, or pharmaceutically acceptable salt thereof, wherein R^{A2} is hydrogen or unsubstituted C₁₋₆ alkyl.

16. The compound of any one of claims 3-15, or pharmaceutically acceptable salt thereof, wherein R^{A2} is hydrogen, methyl, ethyl, n-propyl, or isopropyl.

17. The compound of any one of claims 3-14, or pharmaceutically acceptable salt thereof, wherein R^{A2} is C₁₋₆ alkyl substituted with one or more groups selected from —COOMe, —COOEt, fluoro, chloro, bromo, iodo, —NH₂, —NMe₂, —NEt₂, —OMe, —OEt, and phenyl.

18. The compound of any one of claims 3-14, or pharmaceutically acceptable salt thereof, wherein R^{A2} is cyclopentyl or cyclohexyl.

19. The compound of any one of claims 3-14, or pharmaceutically acceptable salt thereof, wherein R^{A2} is phenyl substituted with one or more groups selected from —COOMe, —COOEt, fluoro, chloro, bromo, iodo, —NH₂, —NMe₂, —NEt₂, —OMe, —OEt, and phenyl.

20. The compound of any one of claims 3-9, or pharmaceutically acceptable salt thereof, wherein R^{A1} and R^{A2} are joined with the intervening nitrogen atom to form pyrrolinyl, piperidinyl, pyrrolyl, morpholinyl, piperazinyl, N-methyl piperazinyl, or thiomorpholinyl.

21. The compound of claim 1 or 3, or pharmaceutically acceptable salt thereof, wherein the compound is of formula: 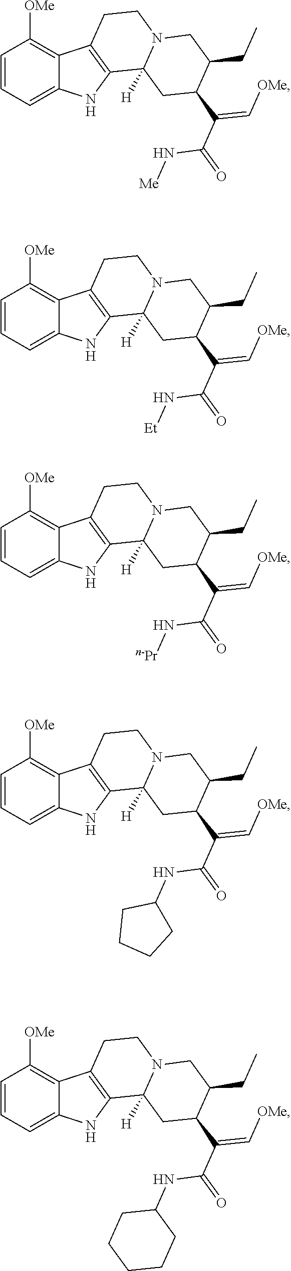 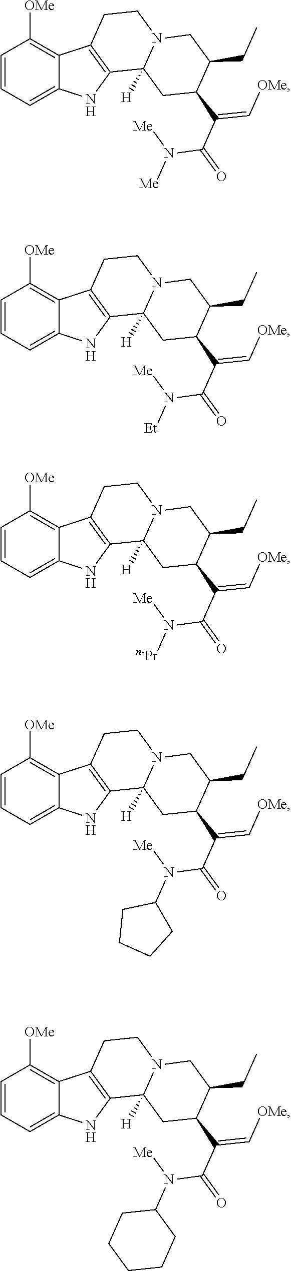 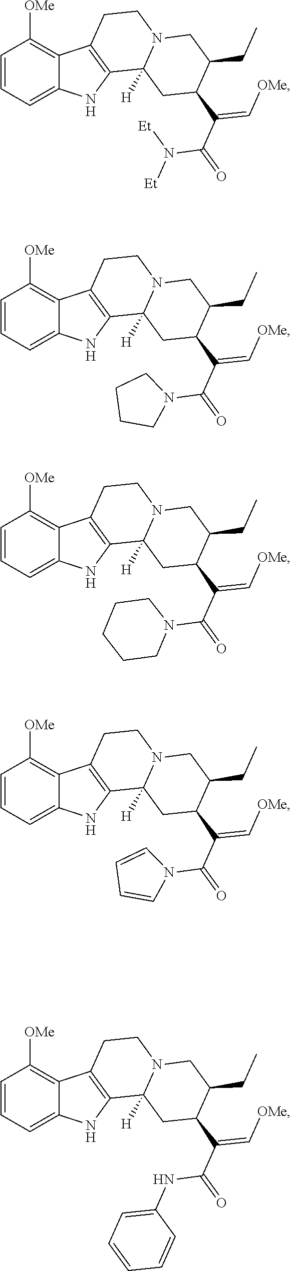 [SMILES: CC[C@@H]1CN2CCc3c([nH]c4cccc(OC)c34)[C@@H]2C[C@@H]1/C(=C\OC)C(=O)N(CC)CC ; CC[C@@H]1CN2CCc3c([nH]c4cccc(OC)c34)[C@@H]2C[C@@H]1/C(=C\OC)C(=O)N1CCCC1 ; CC[C@@H]1CN2CCc3c([nH]c4cccc(OC)c34)[C@@H]2C[C@@H]1/C(=C\OC)C(=O)N1CCCCC1 ; CC[C@@H]1CN2CCc3c([nH]c4cccc(OC)c34)[C@@H]2C[C@@H]1/C(=C\OC)C(=O)n1cccc1 ; CC[C@@H]1CN2CCc3c([nH]c4cccc(OC)c34)[C@@H]2C[C@@H]1/C(=C\OC)C(=O)Nc1ccccc1] 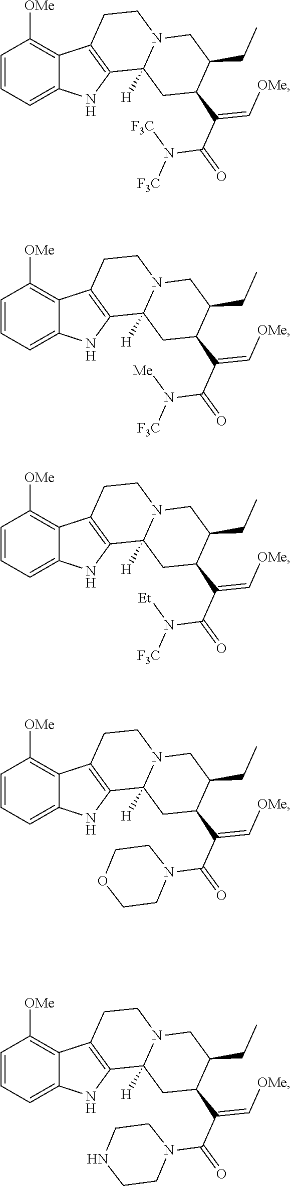 [SMILES: CC[C@@H]1CN2CCc3c([nH]c4cccc(OC)c34)[C@@H]2C[C@@H]1/C(=C\OC)C(=O)N(C(F)(F)F)C(F)(F)F ; CC[C@@H]1CN2CCc3c([nH]c4cccc(OC)c34)[C@@H]2C[C@@H]1/C(=C\OC)C(=O)N(C)C(F)(F)F ; CC[C@@H]1CN2CCc3c([nH]c4cccc(OC)c34)[C@@H]2C[C@@H]1/C(=C\OC)C(=O)N(CC)C(F)(F)F ; CC[C@@H]1CN2CCc3c([nH]c4cccc(OC)c34)[C@@H]2C[C@@H]1/C(=C\OC)C(=O)N1CCOCC1 ; CC[C@@H]1CN2CCc3c([nH]c4cccc(OC)c34)[C@@H]2C[C@@H]1/C(=C\OC)C(=O)N1CCNCC1] 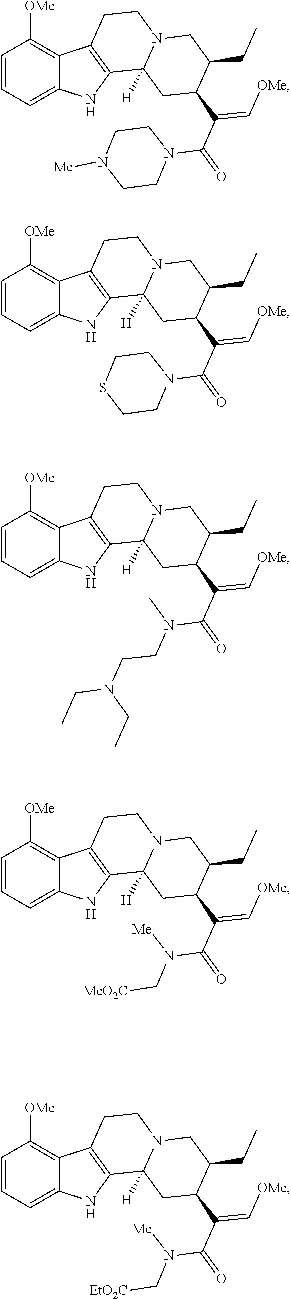 [SMILES: CC[C@@H]1CN2CCc3c([nH]c4cccc(OC)c34)[C@@H]2C[C@@H]1/C(=C\OC)C(=O)N1CCN(C)CC1 ; CC[C@@H]1CN2CCc3c([nH]c4cccc(OC)c34)[C@@H]2C[C@@H]1/C(=C\OC)C(=O)N1CCSCC1 ; CC[C@@H]1CN2CCc3c([nH]c4cccc(OC)c34)[C@@H]2C[C@@H]1/C(=C\OC)C(=O)N(C)CCN(CC)CC ; CC[C@@H]1CN2CCc3c([nH]c4cccc(OC)c34)[C@@H]2C[C@@H]1/C(=C\OC)C(=O)N(C)CC(=O)OC ; CCOC(=O)CN(C)C(=O)/C(=C/OC)[C@H]1C[C@H]2c3[nH]c4cccc(OC)c4c3CCN2C[C@H]1CC] 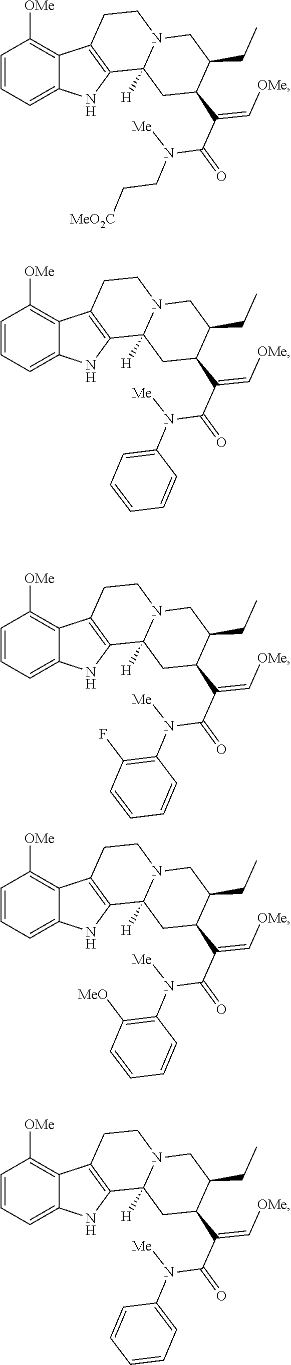 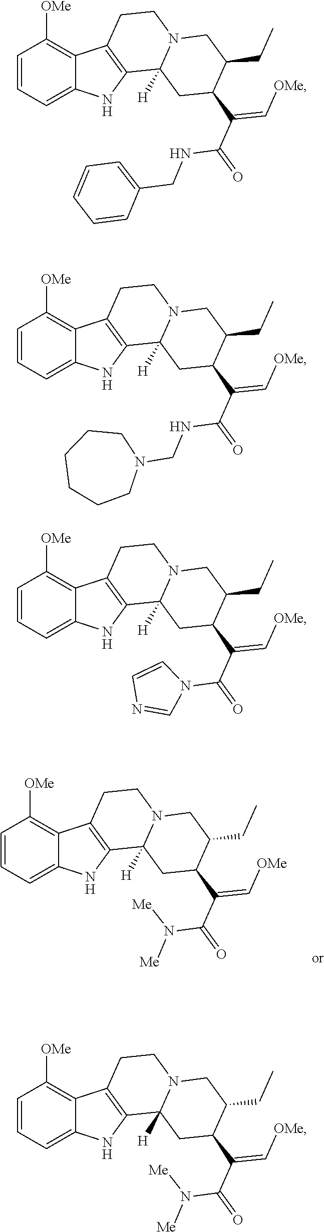 [SMILES: CC[C@@H]1CN2CCc3c([nH]c4cccc(OC)c34)[C@@H]2C[C@@H]1/C(=C\OC)C(=O)NCc1ccccc1 ; CC[C@@H]1CN2CCc3c([nH]c4cccc(OC)c34)[C@@H]2C[C@@H]1/C(=C\OC)C(=O)NCN1CCCCCC1 ; CC[C@@H]1CN2CCc3c([nH]c4cccc(OC)c34)[C@@H]2C[C@@H]1/C(=C\OC)C(=O)n1ccnc1 ; CC[C@H]1CN2CCc3c([nH]c4cccc(OC)c34)[C@@H]2C[C@@H]1/C(=C\OC)C(=O)N(C)C ; CC[C@H]1CN2CCc3c([nH]c4cccc(OC)c34)[C@H]2C[C@@H]1/C(=C\OC)C(=O)N(C)C]
or pharmaceutically acceptable salt thereof.

22. A compound of Formula (II): 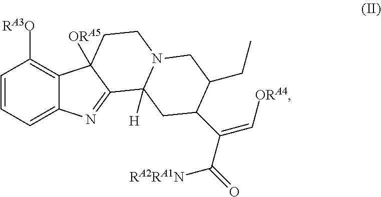
or pharmaceutically acceptable salt thereof, wherein:
R^{A1} is hydrogen, substituted or unsubstituted C₁₋₁₂ alkyl, substituted or unsubstituted 3- to 13-memberedcycloalkyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
R^{A2} is hydrogen, substituted or unsubstituted C₁₋₁₂ alkyl, substituted or unsubstituted 3- to 13-memberedcycloalkyl, substituted or unsubstituted 3- to 13-membered heterocyclyl, substituted or unsubstituted 6- to 12-membered aryl ring, substituted or unsubstituted 5- to 14-membered heteroaryl ring, or a nitrogen protecting group;
or R^{A1} and R^{A2} are joined with the intervening nitrogen atom to form a 3- to 13-membered heterocyclyl or 3- to 13-membered heteroaryl, wherein the heterocyclyl or heteroaryl is substituted or unsubstituted;
R^{A3} is hydrogen, substituted or unsubstituted C₁₋₁₂ alkyl, or an oxygen protecting group;
R^{A4} is hydrogen, substituted or unsubstituted C₁₋₁₂ alkyl, or an oxygen protecting group; and
R^{A5} is hydrogen, substituted or unsubstituted C₁₋₁₂ alkyl, or an oxygen protecting group.

23. The compound of claim 22, or pharmaceutically acceptable salt thereof, wherein the compound of Formula (II) is of Formula (II-A): 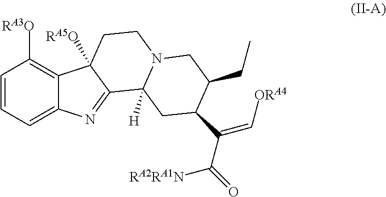
or pharmaceutically acceptable salt thereof.

24. The compound of claim 22 or 23, or pharmaceutically acceptable salt thereof, wherein R^{A3} is methyl.

25. The compound of any one of claims 22-24, or pharmaceutically acceptable salt thereof, wherein R^{A4} is methyl.

26. The compound of any one of claims 22-25, or pharmaceutically acceptable salt thereof, wherein R^{A5} is methyl.

27. The compound of any one of claims 22-26, or pharmaceutically acceptable salt thereof, wherein R^{A5} is hydrogen.

28. The compound of claim 22, or pharmaceutically acceptable salt thereof, wherein the compound is of the formula: 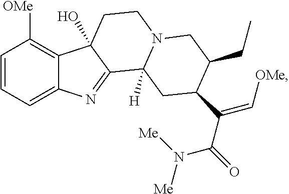 [SMILES: CC[C@@H]1CN2CC[C@@]3(O)C(=Nc4cccc(OC)c43)[C@@H]2C[C@@H]1/C(=C\OC)C(=O)N(C)C]
or pharmaceutically acceptable salt thereof.

29. A compound of Formula (III): 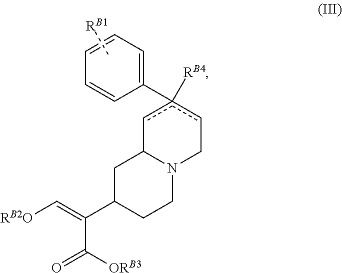
or a pharmaceutically acceptable salt thereof, wherein:
R^{B1} is hydrogen, halo, —OH, —O(C₁₋₆ alkyl), or C₁₋₆ haloalkyl;
R^{B2} is hydrogen, substituted or unsubstituted C₁₋₁₂ alkyl, or an oxygen protecting group;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

30. The compound of claim 29, or pharmaceutically acceptable salt thereof, wherein the compound is of formula: 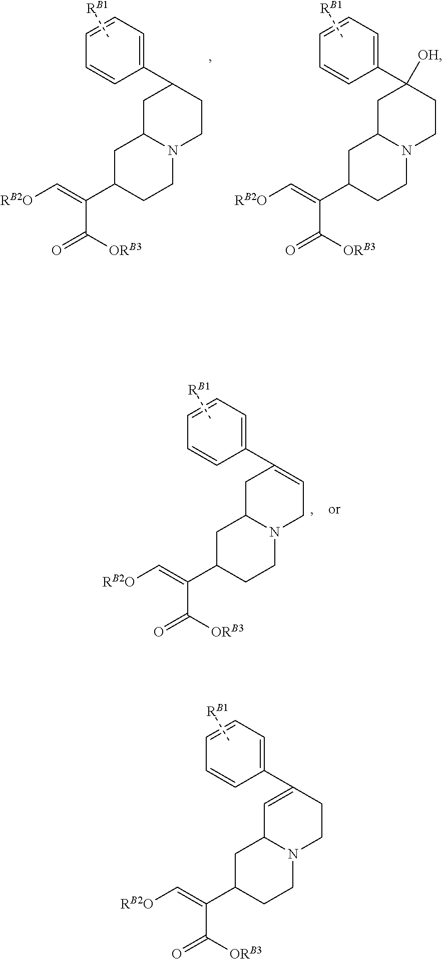
or a pharmaceutically acceptable salt thereof.

31. A compound of Formula (IV): 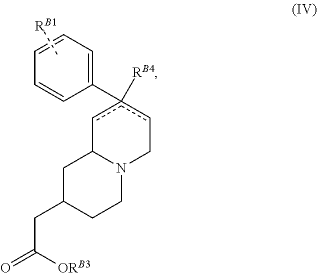
or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

32. The compound of claim 31, or pharmaceutically acceptable salt thereof, wherein the compound is of formula: 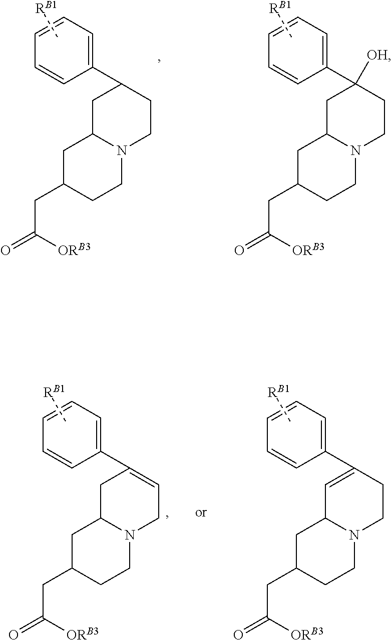
or a pharmaceutically acceptable salt thereof.

33. A compound of Formula (V): 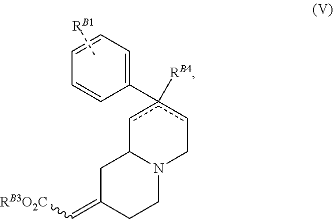
or a pharmaceutically acceptable salt thereof, wherein:
R^{B1} is hydrogen, halo, —OH, —O(C₁₋₆ alkyl), or C₁₋₆ haloalkyl;
R^{B3} is hydrogen, substituted or unsubstituted C₁₋₁₂ alkyl, or an oxygen protecting group;
R^{B4} is hydrogen, —OH, —OMe, —OEt, or absent, as valency permits; and
each instance of [symbol] is either a single or double bond.

34. The compound of claim 33, or pharmaceutically acceptable salt thereof, wherein the compound is of formula: 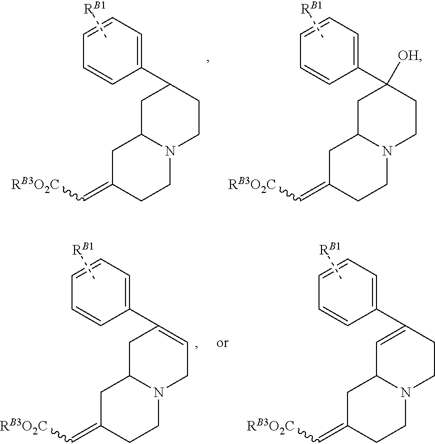
or a pharmaceutically acceptable salt thereof.

35. The compound of any one of claims 30-34, or pharmaceutically acceptable salt thereof, wherein R^{B1} is hydrogen, fluoro, or —OMe.

36. The compound of any one of claims 30-35, or pharmaceutically acceptable salt thereof, wherein R^{B1} is —OMe.

37. The compound of any one of claims 30-36, or pharmaceutically acceptable salt thereof, wherein R^{B1} is ortho —OMe.

38. The compound of any one of claims 30-37, or pharmaceutically acceptable salt thereof, wherein R^{B3} is hydrogen or C₁₋₄ alkyl.

39. The compound of any one of claims 30-38, or pharmaceutically acceptable salt thereof, wherein R^{B3} is methyl.

40. The compound of any one of claims 30-39, or pharmaceutically acceptable salt thereof, wherein R^{B4} is hydrogen or —OH.

41. The compound of any one of claims 30-40, or pharmaceutically acceptable salt thereof, wherein R^{B4} is hydrogen.

42. The compound of any one of claims 30-40, or pharmaceutically acceptable salt thereof, wherein R^{B4} is —OH.

43. The compound of any one of claims 30-39, or pharmaceutically acceptable salt thereof, wherein R^{B4} is absent.

44. The compound of any one of claims 29-43, or pharmaceutically acceptable salt thereof, wherein R^{B2} is hydrogen or C₁₋₄ alkyl.

45. The compound of any one of claims 29-43, or pharmaceutically acceptable salt thereof, wherein R^{B2} is methyl.

46. The compound of claim 29, or pharmaceutically acceptable salt thereof, wherein the compound is of formula: 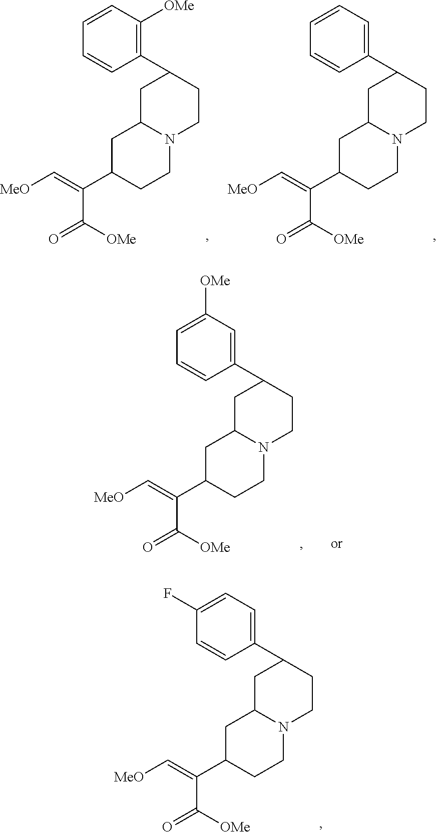 [SMILES: CO/C=C(\C(=O)OC)C1CCN2CCC(c3ccccc3OC)CC2C1 ; CO/C=C(\C(=O)OC)C1CCN2CCC(c3ccccc3)CC2C1 ; CO/C=C(\C(=O)OC)C1CCN2CCC(c3cccc(OC)c3)CC2C1 ; CO/C=C(\C(=O)OC)C1CCN2CCC(c3ccc(F)cc3)CC2C1]
or a pharmaceutically acceptable salt thereof.

47. The compound of claim 31 or pharmaceutically acceptable salt thereof, wherein the compound is of formula: 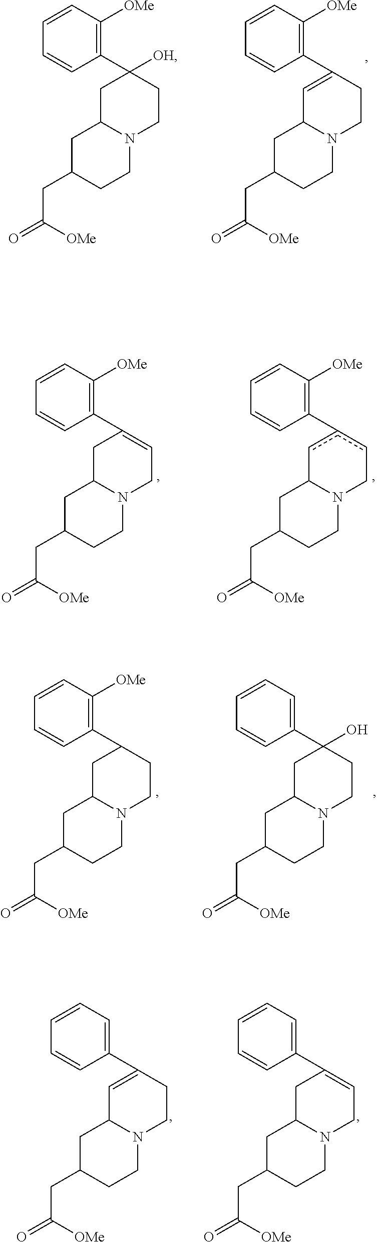 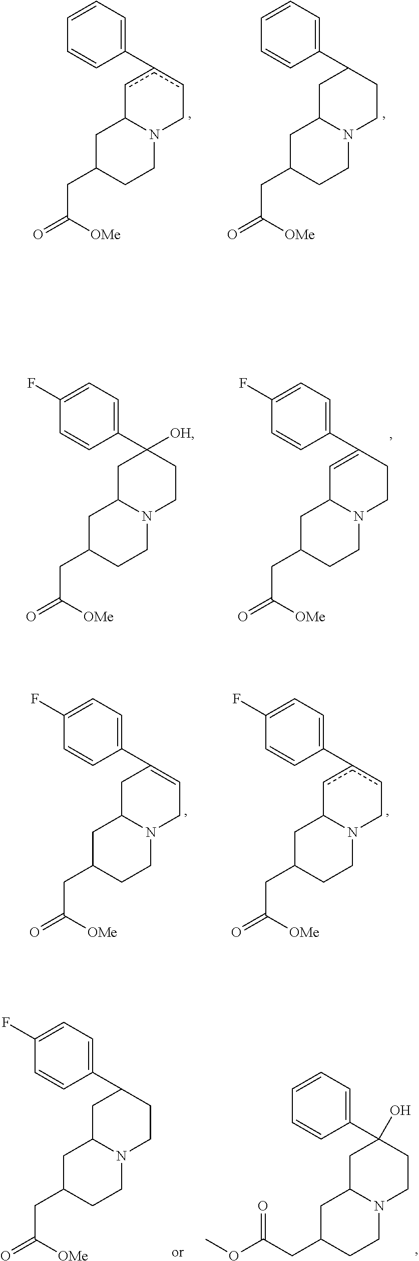
or a pharmaceutically acceptable salt thereof.

48. The compound of claim 33, or pharmaceutically acceptable salt thereof, wherein the compound is of formula: 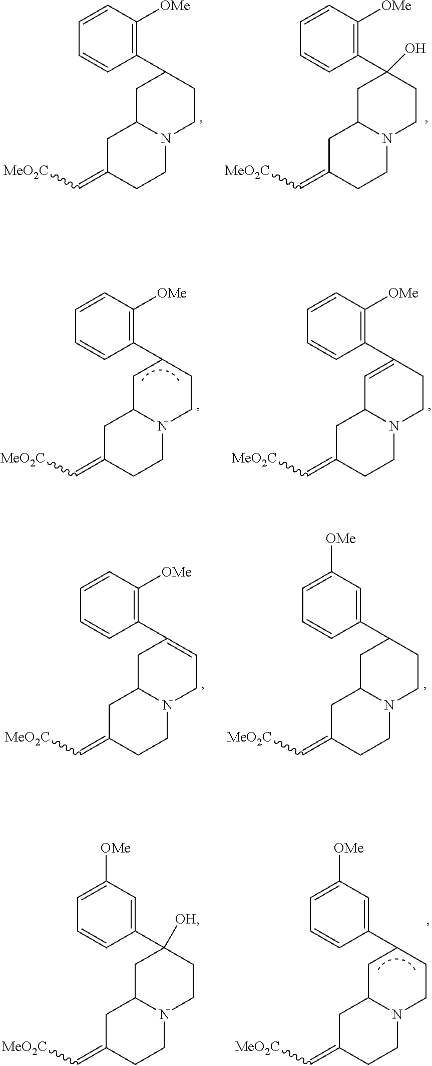 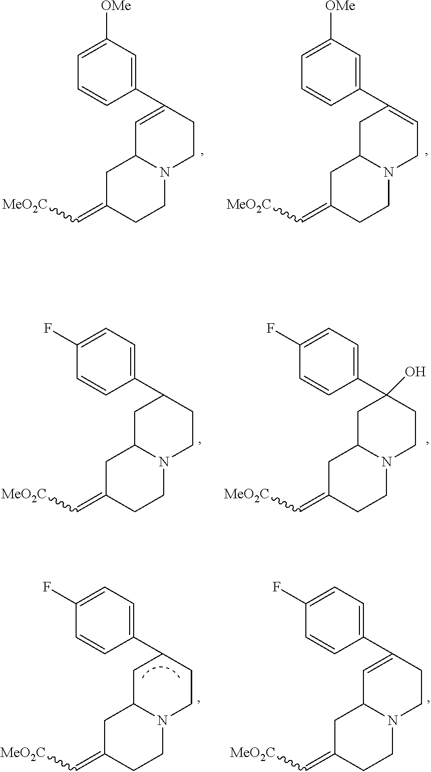 [SMILES: COC(=O)C=C1CCN2CCC(c3cccc(OC)c3)=CC2C1 ; COC(=O)C=C1CCN2CC=C(c3cccc(OC)c3)CC2C1 ; COC(=O)C=C1CCN2CCC(c3ccc(F)cc3)CC2C1 ; COC(=O)C=C1CCN2CCC(O)(c3ccc(F)cc3)CC2C1 ; COC(=O)C=C1CCN2CCC(c3ccc(F)cc3)CC2C1 ; COC(=O)C=C1CCN2CCC(c3ccc(F)cc3)=CC2C1] 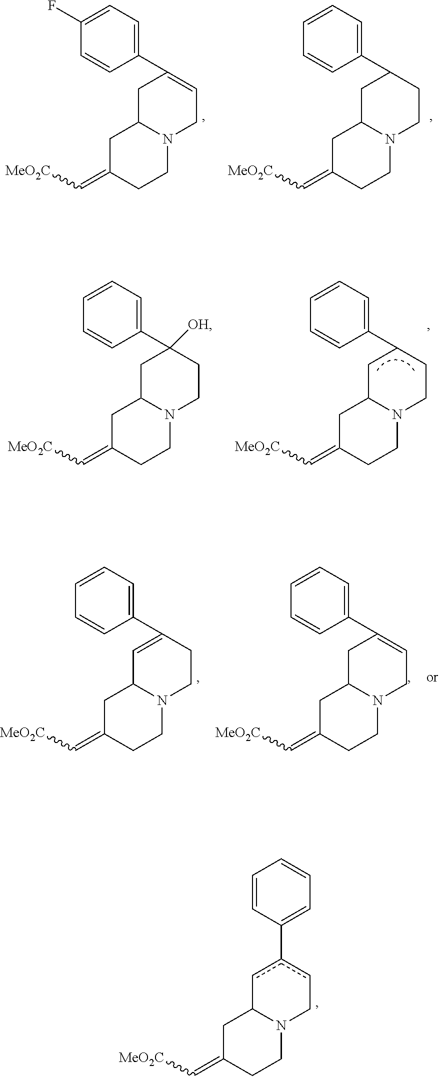
or a pharmaceutically acceptable salt thereof.

49. A compound of Formula (VI): 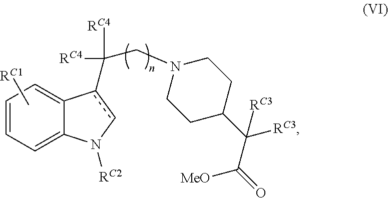
or pharmaceutically acceptable salt thereof, wherein:
R^{C1} is hydrogen, —OH, or —OMe;
R^{C2} is hydrogen, unsubstituted C₁₋₆ alkyl, or C₁₋₆ alkyl substituted with one or more instances of —OH, —OMe, fluoro, indolyl;
each instance of RC³ is hydrogen or both instances of R^{3C} are taken together to form: 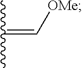 [SMILES: *=COC]
each instance of R^{C4} is hydrogen or both instances of R^{C4} are taken together to form: ═O; and
n is 0, 1, or 2.

50. The compound of claim 49, or pharmaceutically acceptable salt thereof, wherein the compound is of formula: 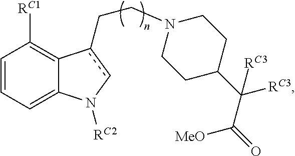
or pharmaceutically acceptable salt thereof.

51. The compound of claim 49, or pharmaceutically acceptable salt thereof, wherein the compound is of formula: 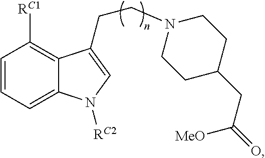
or pharmaceutically acceptable salt thereof.

52. The compound of claim 49, or pharmaceutically acceptable salt thereof, wherein the compound is of formula: 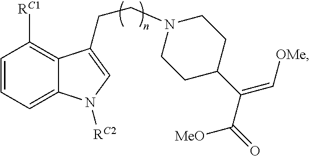
or pharmaceutically acceptable salt thereof.

53. The compound of any one of claims 49-52, or pharmaceutically acceptable salt thereof, wherein R^{C1} is —OMe.

54. The compound of any one of claims 49-53, or pharmaceutically acceptable salt thereof, wherein R^{C2} is hydrogen or C₁₋₆ alkyl substituted with one or more instances of fluoro or indolyl.

55. The compound of any one of claims 49-54, or pharmaceutically acceptable salt thereof, wherein n is 1.

56. The compound of any one of claims 49-54, or pharmaceutically acceptable salt thereof, wherein n is 2.

57. The compound of any one of claims 49-54, or pharmaceutically acceptable salt thereof, wherein n is 0.

58. The compound of claim 49, or pharmaceutically acceptable salt thereof, wherein the compound is of formula:

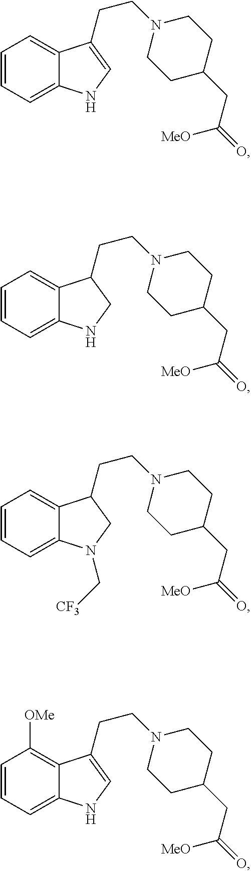 [SMILES: COC(=O)CC1CCN(CCc2c[nH]c3ccccc23)CC1 ; COC(=O)CC1CCN(CCC2CNc3ccccc32)CC1 ; COC(=O)CC1CCN(CCC2CN(CC(F)(F)F)c3ccccc32)CC1 ; COC(=O)CC1CCN(CCc2c[nH]c3cccc(OC)c23)CC1]

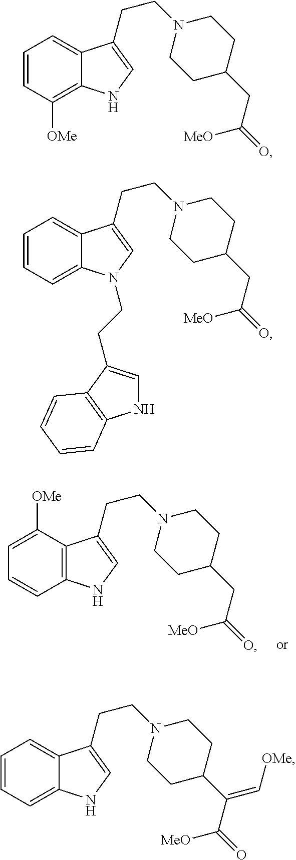 [SMILES: COC(=O)CC1CCN(CCc2c[nH]c3c(OC)cccc23)CC1 ; COC(=O)CC1CCN(CCc2cn(CCc3c[nH]c4ccccc34)c3ccccc23)CC1 ; COC(=O)CC1CCN(CCc2c[nH]c3cccc(OC)c23)CC1 ; CO/C=C(/C(=O)OC)C1CCN(CCc2c[nH]c3ccccc23)CC1]

59. A compound of Formula (VII): 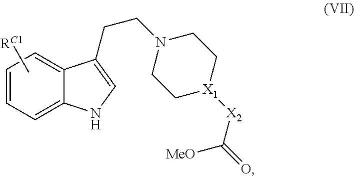
or pharmaceutically acceptable salt thereof, wherein:
R^{C1} is hydrogen, —OH, or —OMe;
X₁ is CH or N; and
X₂ is NH, CH₂, or CH—(CH₂)—OMe.

60. The compound of claim 59, or pharmaceutically acceptable salt thereof, wherein R^{C1} is hydrogen or —OMe.

61. The compound of claim 59 and 60, or pharmaceutically acceptable salt thereof, wherein X₁ is N and X₂ is CH₂.

62. The compound of claim 59 and 60, or pharmaceutically acceptable salt thereof, wherein X₁ is CH and X₂ is NH.

63. The compound of claim 59 and 60, or pharmaceutically acceptable salt thereof, wherein X₁ is CH and X₂ is CH—(CH₂)—OMe.

64. The compound of claim 59, or pharmaceutically acceptable salt thereof, wherein the compound is of formula: 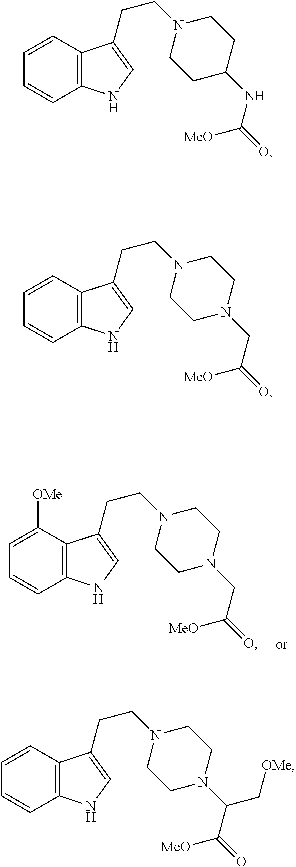 [SMILES: COC(=O)NC1CCN(CCc2c[nH]c3ccccc23)CC1 ; COC(=O)CN1CCN(CCc2c[nH]c3ccccc23)CC1 ; COC(=O)CN1CCN(CCc2c[nH]c3cccc(OC)c23)CC1 ; COCC(C(=O)OC)N1CCN(CCc2c[nH]c3ccccc23)CC1]
or pharmaceutically acceptable salt thereof.

65. A compound of Formula (VIII): 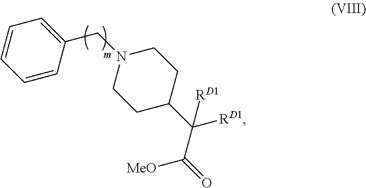
or pharmaceutically acceptable salt thereof, wherein:
m is 1, 2, or 3; and
each instance of R^{D1} is hydrogen or both instances of R^{D1} are taken together to form:

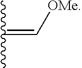 [SMILES: *=COC]

66. A compound of Formula (IX): 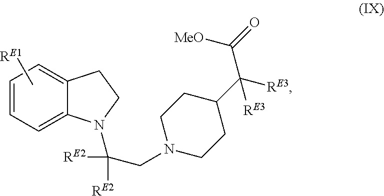
or pharmaceutically acceptable salt thereof, wherein:
R^{E1} is hydrogen, —OH, or —OMe;
each instance of R^{E2} is hydrogen or both instances of R^{E2} are taken together to form: ═O; and
each instance of R^{E3} is hydrogen or both instances of R^{E3} are taken together to form:

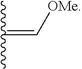 [SMILES: *=COC]

67. The compound of claim 66, or pharmaceutically acceptable salt thereof, wherein the compound is of formula: 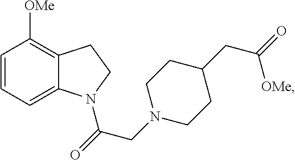 [SMILES: COC(=O)CC1CCN(CC(=O)N2CCc3c(OC)cccc32)CC1]
or pharmaceutically acceptable salt thereof.

68. The compound of any one of claims 1-67, or pharmaceutically acceptable salt thereof, wherein the compound is an opioid receptor binder.

69. The compound of any one of claims 1-68, or pharmaceutically acceptable salt thereof, wherein the compound is an α₂ adrenergic receptor binder.

70. A composition comprising the compound of any one of claims 1-69, or pharmaceutically acceptable salt thereof, and a pharmaceutically acceptable excipient.

71. A method of treating or preventing substance intake by a subject comprising administering to the subject a therapeutically effective amount of the compound of any one of claims 1-69, or pharmaceutically acceptable salt thereof, or composition of claim 70.

72. A method of treating or preventing substance use disorder in a subject comprising administering to the subject a therapeutically effective amount of the compound of any one of claims 1-69, or pharmaceutically acceptable salt thereof, or composition of claim 70.

73. A method of treating the symptoms of substance use disorder in a subject comprising administering to the subject a therapeutically effective amount of the compound of any one of claims 1-69, or pharmaceutically acceptable salt thereof, or composition of claim 70.

74. A method of treating or preventing substance addiction in a subject comprising administering to the subject a therapeutically effective amount of the compound of any one of claims 1-69, or pharmaceutically acceptable salt thereof, or composition of claim 70.

75. A method of treating the symptoms of substance addiction in a subject comprising administering to the subject a therapeutically effective amount of the compound of any one of claims 1-69, or pharmaceutically acceptable salt thereof, or composition of claim 70.

76. The method of any one of claims 71-75, wherein the substance is an opioid.

77. The method of any one of claims 71-76, wherein the substance is a prescription opioid.

78. The method of any one of claims 71-77, wherein the substance is codeine, fentanyl, hydromorphone, meperidine, methadone, morphine, oxycodone, or oxymorphone.

79. The method of any one of claims 71-76, wherein the substance is an illicit opioid.

80. The method of any one of claims 71-76 and 79, wherein the substance is heroin.

81. A method of treating or preventing neurotoxic effects resulting from substance use disorder, substance addiction, and/or substance intake by a subject comprising administering to the subject a therapeutically effective amount of the compound of any one of claims 1-69, or pharmaceutically acceptable salt thereof, or composition of claim 70.

82. A method of treating or preventing a disease or disorder comprising administering to a subject a therapeutically effective amount of the compound of any one of claims 1-69, or pharmaceutically acceptable salt thereof, or composition of claim 70.

83. The method of claim 82, wherein the disease or disorder is associated with opioid receptors and/or adrenergic receptors.

84. A method of treating or preventing a disease or disorder associated with one or more opioid receptors comprising administering to a subject a therapeutically effective amount of the compound of any one of claims 1-69, or pharmaceutically acceptable salt thereof, or composition of claim 70.

85. The method of claim 83 or 84, wherein the opioid receptor is a kappa opioid receptor, delta opioid receptor, and/or a mu opioid receptor.

86. The compound of claim 68, wherein the opioid receptor is a kappa opioid receptor, delta opioid receptor, and/or a mu opioid receptor.

87. A method of treating or preventing a disease or disorder associated with one or more adrenergic receptors comprising administering to a subject a therapeutically effective amount of the compound of any one of claims 1-69, or pharmaceutically acceptable salt thereof, or composition of claim 70.

88. The method of claim 83 or 87, wherein the adrenergic receptor is an alpha-2 receptor.

89. The method of any one of claims 83, 87, or 88, wherein the adrenergic receptor is α2A and/or α2C receptor.

90. The method of any one of claims 82-89, wherein the disease or disorder is a psychiatric disorder, a painful condition, or a neurological disorder.

91. The method of any one of claims 82-90, wherein the disease or disorder is depression, a mood disorder, nociceptive pain, neuropathic pain, or pain associated with withdrawal symptoms from substance addiction.

92. A method of treating or preventing substance withdrawal comprising administering to a subject a therapeutically effective amount of the compound of any one of claims-1-69, or pharmaceutically acceptable salt thereof, or composition of claim 70.

93. A method of treating or preventing symptoms associated with substance withdrawal comprising administering to a subject a therapeutically effective amount of the compound of any one of claims 1-69, or pharmaceutically acceptable salt thereof, or composition of claim 70.

94. A method of treating or preventing substance dependence comprising administering to a subject a therapeutically effective amount of the compound of any one of claims 1-69, or pharmaceutically acceptable salt thereof, or composition of claim 70.

95. The method of any one of claims 92-94, wherein the substance is an opioid.

96. The method of any one of claims 92-95, wherein the substance is a prescription opioid.

97. The method of any one of claims 92-96, wherein the substance is codeine, fentanyl, hydromorphone, meperidine, methadone, morphine, oxycodone, or oxymorphone.

98. The method of any one of claims 92-95, wherein the substance is an illicit opioid.

99. The method of any one of claims 92-94 and 98, wherein the substance is heroin.

100. A kit comprising:
the compound of any one of claims 1-69, or pharmaceutically acceptable salt thereof, or composition of claim 70; and
and instructions for administering the compound, or pharmaceutically acceptable salt thereof, or composition to a subject.
